(19) United States
(12) Patent Application Publication
(10) Pub. No.: US 2023/0303713 A1
(43) Pub. Date: Sep. 28, 2023

(54) ANTI-CD19 CAR-T CELLS WITH MULTIPLE GENE EDITS AND THERAPEUTIC USES THEREOF

(71) Applicant: CRISPR Therapeutics AG, Zug (CH)

(72) Inventors: Mohammed Ghonime, Cambridge, MA (US); Jonathan Alexander TERRETT, Cambridge, MA (US); Demetrios KALAITZIDIS, Cambridge, MA (US); Mary-Lee Dequeant, Cambridge, MA (US)

(21) Appl. No.: 18/188,436

(22) Filed: Mar. 22, 2023

Related U.S. Application Data
(60) Provisional application No. 63/322,903, filed on Mar. 23, 2022.

(51) Int. Cl. C07K 16/28; A61K 35/17; A61P 35/00; C12N 15/86

(52) U.S. Cl. CPC C07K 16/2896; A61K 35/17; A61P 35/00; C12N 15/86; C12N 2750/14143; C12N 2510/00

(57) ABSTRACT

Genetically engineered T cells expressing a chimeric antigen receptor (CAR) targeting CD19 and having multiple genetic edits, including a disrupted TRAC gene, a disrupted β2M gene, a disrupted Regnase 1 gene, and/or a disrupted TGFBRII gene. Also provided herein are methods of making such genetically engineered T cells and methods of using the genetically engineered T cells in cancer treatment.

### CROSS REFERENCE TO RELATED APPLICATIONS

This application claims the benefit of the filing date of U.S. Provisional Application No. 63/322,903, filed Mar. 23, 2022, the entire contents of which is incorporated by reference herein.

### SEQUENCE LISTING

The application contains a Sequence Listing that has been filed electronically in XML format, created Mar. 14, 2023, and named “095136-0740-065US1_SEQ.XML” (101,795 bytes), the contents of which are incorporated by reference herein in their entirety.

### BACKGROUND OF THE INVENTION

Chimeric antigen receptor (CAR) T-cell therapy uses genetically modified T cells to more specifically and efficiently target and kill cancer cells. After T cells have been collected from the blood, the cells are engineered to include CARs on their surface. The CARs may be introduced into the T cells using CRISPR/Cas9 gene editing technology. When these allogeneic CAR T cells are injected into a patient, the receptors enable the T cells to kill cancer cells.

T cells having improved persistence in culture are desired in CAR T therapy. Such T cells live longer in both in vitro and in vivo, thereby conferring benefits in CAR T cell manufacturing and clinical applications. However, it remains challenging to improve persistence of T cells in culture.

### SUMMARY OF THE INVENTION

The present disclosure is based, at least in part, on the development of genetically edited anti-CD19 CAR-T cells carrying a disrupted Regnase 1 (Reg1) gene, a disrupted TGFBRII gene, a disrupted TRAC gene, and/or a disrupted β2M gene (e.g., anti-CD19 CAR-T cells carrying all of the disrupted genes), effective methods of producing such genetically edited T cells via CRISPR/Cas-mediated gene editing using guide RNAs as disclosed herein. It was reported herein that the disruption of both the Reg1 gene and the TGFBRII gene showed synertistic effect in increasing CAR-T cell expansion and functional persistence, leading to enhanced therapeutic efficacy against CD19+ cancer both in vitro and in vivo. For example, the anti-CD19 CAR-T cells enhanced survival rates as observed in leukemia and lymphoma animal models. Further, the anti-CD19 CAR-T cells with both Reg1 and TGFBRII disruption showed therapeutic effectiveness at doses lower than the anti-CD19 CAR-T cell counterparts with wild-type Reg1 and TGFBRII.

Accordingly, provided herein, in some aspects, is a population of genetically engineered T cells, comprising: (i) a disrupted T cell receptor alpha chain constant region (TRAC) gene, (ii) a disrupted beta-2-microglobulin (β2M) gene, (iii) a disrupted Regnase-1 (Reg1) gene, (iv) a disrupted Transforming Growth Factor Beta Receptor II (TGFBRII) gene, and (v) a nucleic acid encoding a chimeric antigen receptor (CAR) that binds human CD19 (anti-CD19 CAR). In some embodiments, the anti-CD19 CAR comprises a single chain variable fragment (scFv) that binds CD19 (anti-CD19 scFv), a co-stimulatory domain of CD28, and a CD3ζ cytoplasmic signaling domain.

In some instances, the anti-CD19 scFv in the anti-CD19 CAR may comprise a heavy chain variable region (V_{H}) that comprises the same heavy chain complementary determining regions (CDRs) as those in SEQ ID NO: 81; and (ii) a light chain variable region (VL) that comprises the same light chain CDRs as those in SEQ ID NO: 82. In some instances, the anti-CD19 scFv comprises the VH comprising the amino acid sequence of SEQ ID NO: 81 and the VL comprising the amino acid sequence of SEQ ID NO: 82. In specific examples, the anti-CD19 scFv comprises the amino acid sequence of SEQ ID NO: 77. In one example, the anti-CD19 CAR comprises the amino acid sequence of SEQ ID NO: 74.

In some instances, the nucleic acid encoding the anti-CD19 CAR is inserted at the disrupted TRAC gene. For example, a fragment comprising the nucleotide sequence of SEQ ID NO: 18 in the TRAC gene may be deleted and replaced by the nucleic acid encoding the anti-CD19 CAR. In some examples, the disrupted TRAC gene comprises the nucleotide sequence of SEQ ID NO: 91.

In some embodiments, the disrupted β2M gene in the T cells comprises one or more of the nucleotide sequences listed in Table 2. In some embodiments, the disrupted Reg1 gene in the T cells comprises one or more of the nucleotide sequences listed in Table 4. Alternatively or in addition, the disrupted TGFBRII gene in the T cells comprises one or more of the nucleotide sequences listed in Table 3.

In some embodiments, the population of the genetically engineered T cells comprises at least 50% CAR+ cells, at least 90% TCR⁻ T cells, at least 60% β2M⁻ T cells, at least 80% TGFBRII⁻ T cells, and/or at least 90% Reg1⁻ T cells. In some examples, the population of the genetically engineered T cells comprises at least 75% CAR+ T cells, at least 99% TCR⁻ T cells are, about 65% to about 80% of β2M⁻ T cells; about 80% to about 90% of TGFBRII⁻ T cells, and/or about 95% to about 97% of Reg1⁻ T cells.

In some embodiments, the population of genetically engineered T cells as disclosed herein comprise primary human T cells. In some instances, the primary human T cells are derived from one or more healthy human donors.

In other aspects, provided herein is a method for treating a CD19⁺ cancer, comprising administering to a subject in need thereof an effective amount of any of the populations of genetically engineered T cells as disclosed herein. In some embodiments, the subject is a human patient having a B cell malignancy, for example, a refractory or relapsed B cell malignancy. Exemplary B cell malignancies include, but are not limited to, non-Hodgkin lymphoma, which optionally is selected from the group consisting of diffuse large B cell lymphoma (DLBCL), which optionally is DLBCL not otherwise specified (NOS), high grade B cell lymphoma with MYC and BCL2 and/or BCL6 rearrangement, transformed follicular lymphoma (FL), and grade 3b FL. In some examples, the effective amount of the population of genetically engineered T cells range from about 1×10⁷ to about 6×10⁸ CAR⁺ T cells, for example, about 1×10⁷ to about 3×10⁷ CAR+ T cells, about 3×10⁷ to about 1×10⁸ CAR+ T cells, about about 1×10⁸ to about 3×10⁸ CAR+ T cells, or about 3×10⁸ to about 6×10⁸ CAR+ T cells.

Further, provided herein is a method for preparing a population of genetically engineered T cells of as disclosed herein. The method may comprise: (a) providing a plurality of cells, which are T cells or precursor cells thereof; (b) genetically editing the TRAC gene, the β2M gene, the Reg1 gene, and the TGFBRII gene of the plurality of cells; and (c) delivering the nucleic acid encoding the anti-CD19 CAR into the plurality of cells, thereby producing the population of genetically engineered T cells. In some instances, the nucleic acid encoding the anti-CD19 CAR inserts into the TRAC gene.

In some instances, the plurality of cells in step (a) are primary human T cells. For example, the primary human T cells may be from one or more healthy human donors.

In some instances, the genetic editing of the TRAC gene, the β2M gene, the Reg1 gene, and the TGFBRII gene is performed by one or more CRISPR/Cas9 gene editing systems. In some examples, the one or more CRISPR/Cas9 gene editing system comprises: (a) an RNA-guided nuclease, (b) a TRAC-targeting guide RNA comprising a spacer of SEQ ID NO: 3, (c) a β2M-targeting guide RNA comprising a spacer of SEQ ID NO: 7, (d) a Reg1-targeting guide RNA comprising a spacer of SEQ ID NO: 11, and (e) a TGFBRII-targeting guide RNA comprising a spacer of SEQ ID NO: 15. In some instances, the RNA-guided nuclease may be a Cas9 nuclease, for example, a S. pyogenes Cas9 nuclease.

In some instances, the RNA-guided nuclease and the guide RNAs of (b)-(e) form one or more one or more ribonucleoprotein particles, which are delivered to the plurality of cells by one or more electroporation events.

In some instances, the nucleic acid encoding the anti-CD19 CAR is in an AAV vector. In some examples, the nucleic acid encoding the anti-CD19 CAR comprises a left homology arm and a right homology arm flanking the nucleotide sequence encoding the CAR. The left homology arm and the right homology arm are homologous to a genomic locus in the TRAC gene, allowing for insertion of the nucleic acid into the genomic locus.

Any of the genetically engineered T cells produced from any of the methods disclosed herein is also within the scope of the present disclosure.

Also within the scope of the present disclosure are any of the populations of genetically engineered T cells as disclosed herein for use in treating a CD19+ cancer such as those disclosed herein, or for use in manufacturing a medicament for the intended therapeutic purposes as disclosed herein.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

FIGS. 1A-1C illustrate that TGFBRII disruption eliminates TGF-β inhibition on cell expansion by. FIG. 1A: inhibition of the growth of mock-electroporated human T cells by TGF-β. FIG. 1B: elimination of TGF-β inhibition of the growth of T cells edited to lack TGFBRII (along with disruptions in the TRAC, β2M, and Regnase-1 loci) without CAR. FIG. 1C: elimination of TGF-β inhibition of the growth of T cells edited to knock out TGFBRII (along with disruptions in the TRAC, B2M, and Regnase-1 loci) and expressing an anti-CD19 CAR [CAR-T (R/T)], R/T referring to disruption of Reg1 and TGFBRII.

FIGS. 2A-2E illustrate that the anti-CD19 CAR+TRAC KO+β2M KO+R/T KO T cells selectively secrete IFN-γ in the presence of CD19 expression. FIG. 2A: no high-level secretion of IFN-γ in the presence of the CD19-negative K562 cell line. FIG. 2B: secretion of high levels of IFN-γ when CD19 was expressed in K562 cells (K562-CD19). FIG. 2C: secretion of high levels of IFN-γ in human CD19-positive Raji-luciferase lymphoma cells. FIG. 2D: secretion of high levels of IFN-γ in human CD19-positive Nalm6-leukemia cells. FIG. 2E: secretion of high levels of IFN-γ in human CD19-positive Jeko-1 lymphoma cells. CAR-T (R/T) refers to anti-CD19 CAR-T cells with disrupted TRAC, B2M, Reg1, and TGFBRII.

FIGS. 3A-3E illustrate that the anti-CD19 CAR+TRAC KO+β2M KO+R/T KO T cells specifically kill CD19-expressing cells with high levels of cytotoxicity. FIG. 3A: levels of cytotoxic activity against the CD19-negative cell line K562 are not above control cells—mock (TCR⁺ T cells that were mock electroporated) or no CAR (cells containing TRAC, β2M, and R/T edits except for CAR insertion). FIG. 3B: high levels of cytotoxicity in K562 cells engineered to express human CD19 (K562-CD19). FIG. 3C: high levels of cytotoxicity in human CD19-positive Raji-luciferase lymphoma cells. FIG. 3D: high levels of cytotoxicity in human CD19-positive Nalm6-leukemia cells. FIG. 3E: high levels of cytotoxicity in human CD19-positive Jeko-1 lymphoma cells. CAR-T (R/T) refers to anti-CD19 CAR-T cells with disrupted TRAC, B2M, Reg1, and TGFBRII.

FIGS. 4A-4D illustrate that R/T disruptions synergistically increase potency of the anti-CD19 CAR+TRAC KO+β2M KO+R/T KO T cells against CD19-positive malignancies. FIG. 4A: increased survival (probability of survival) of CD19⁺ Nalm6 leukemia mice. FIG. 4B: increased survival (probability of survival) of CD19⁺ Jeko-1 lymphoma mice. FIG. 4C: increased expansion and persistence (CAR copies per μg of DNA) of the anti-CD19 CAR+TRAC KO+β2M KO+R/T KO T cells relative to the anti-CD19 CAR+TRAC KO+β2M KO T cells or the anti-CD19 CAR+TRAC KO+β2M KO+TGFBR2 KO T cells or the anti-CD19 CAR+TRAC KO+β2M KO+Regnase-1 KO T cells in Nalm6 models. FIG. 4D: increased expansion and persistence (CAR copies per μg of DNA) of the anti-CD19 CAR+TRAC KO+β2M KO+R/T KO T cells relative to the anti-CD19 CAR+TRAC KO+β2M KO T cells or the anti-CD19 CAR+TRAC KO+β2M KO+TGFBR2 KO T cells or the anti-CD19 CAR+TRAC KO+β2M KO+Regnase-1 KO T cells in Jeko-1 models. CAR-T refers to anti-CD19 CAR-T cells with disrupted TRAC and B2M. CAR-T (R/T) refers to anti-CD19 CAR-T cells with disrupted TRAC, B2M, Reg1, and TGFBRII.

FIGS. 5A-5B illustrate that polyclonal anti-CD19 CAR T cells with TRAC, β2M, Reg1 and TGFBRII knockouts persist in both female (FIG. 5A) and male (FIG. 5B) mice.

FIGS. 6A-6C illustrate that anti-CD19 CAR+TRAC KO+β2M KO+Reg1 KO+TGFBRII KO T cells control leukemia/lymphoma at low cell doses. FIG. 6A: high level of tumor control in Raji-luciferase lymphoma cells. FIG. 6B: high level of tumor control in Nalm6-leukemia cells. FIG. 6C: high level of tumor control in Jeko-1 lymphoma model at doses lower than a sub-efficacious dose of anti-CD19 CAR T cells with only TRAC and β2M knockouts. BLI: bioluminescence imaging.

FIGS. 7A-7B illustrate that anti-CD19 CAR T cells with TRAC, β2M, Reg1 and TGFBRII knockouts (FIG. 7A) are efficacious in treating Nalm6-leukemia mice at lower doses relative to anti-CD19 CAR T cells with only TRAC and β2M knockouts (FIG. 7B).

FIG. 8 illustrates that anti-CD19 CAR T cells with TRAC, β2M, Reg1, and TGFBRII knockouts require cytokines for cell growth.

FIG. 9 illustrates GLP-compliant tumorigenicity study in NSG mice using anti-CD19 CAR T cells with TRAC, β2M, Reg1, and TGFBRII knockouts. GLP: Good Laboratory Practice; RT: radiation therapy. CAR-T (R/T) refers to anti-CD19 CAR-T cells with disrupted TRAC, B2M, Reg1, and TGFBRII.

FIG. 10 illustrates body weight change in mice treated with both low dose (0.5×10⁶ cells/mouse) and high dose (1×10⁷ cells/mouse) of anti-CD19 CAR T cells with TRAC, β2M, Reg1, and TGFBRII knockouts. CAR-T (R/T) refers to anti-CD19 CAR-T cells with disrupted TRAC, B2M, Reg1, and TGFBRII.

FIG. 11 illustrates exposure of anti-CD19 CART cells with TRAC, β2M, Reg1, and TGFBRII knockouts in mouse blood. CAR-T (R/T) refers to anti-CD19 CAR-T cells with disrupted TRAC, B2M, Reg1, and TGFBRII.

FIG. 12 illustrates that anti-CD19 CAR T cells with TRAC and β2M knockouts elicit comparable allogeneic mixed lymphocyte reaction (MLR) responses in vitro with or without further Reg1 and TGFBRII knockouts. CAR-T refers to anti-CD19 CAR-T cells with disrupted TRAC and B2M. CAR-T (R/T) refers to anti-CD19 CAR-T cells with disrupted TRAC, B2M, Reg1, and TGFBRII.

FIGS. 13A-13B illustrate that allogeneic natural killer (NK) cells from both donor 1 (FIG. 13A) and donor 2 (FIG. 13B) can lyse anti-CD19 CAR T cells with only TRAC and β2M knockouts or anti-CD19 CAR T cells with TRAC, β2M, Reg1 and TGFBRII knockouts in vitro. CAR-T refers to anti-CD19 CAR-T cells with disrupted TRAC and B2M. CAR-T (R/T) refers to anti-CD19 CAR-T cells with disrupted TRAC, B2M, Reg1, and TGFBRII.

FIGS. 14A-14C illustrate comparable response to allogeneic NK and T cell from donor 1 (FIG. 14A), donor 2 (FIG. 14B) and donor 3 (FIG. 14C) in vivo elicited by anti-CD19 CAR T cells with only TRAC and β2M knockouts or anti-CD19 CAR T cells with TRAC, β2M, Reg1 and TGFBRII knockouts. CAR-T refers to anti-CD19 CAR-T cells with disrupted TRAC and B2M. CAR-T (R/T) refers to anti-CD19 CAR-T cells with disrupted TRAC, B2M, Reg1, and TGFBRII.

### DETAILED DESCRIPTION OF THE INVENTION

The present disclosure aims at establishing genetically engineered anti-CD19 CAR-T cells having improved growth activity, persistence, reduced T cell exhaustion, and enhanced potency, a long-felt need in CAR-T therapy. The anti-CD19 CAR-T cells disclosed herein may comprise multiple genetic edits on endogenous genes, for example, disruption of the TRAC gene, the β2M gene, the TGFBRII gene, and the Reg1 gene, to make the cells suitable for allogeneic immune cell therapy and to achieve features that could improve treatment efficacy. For example, β2M disruption can reduce the risk of or prevent a host-versus-graft response and TRAC disruption can reduce the risk of or prevent a graft-versus-host response. In addition, TGFBRII disruption may reduce immunosuppressive effect of transforming growth factor beta (TGF-β) in the tumor microenvironment and Reg1 disruption may improve CAR-T cell functionality via long-term persistence with robust effector function.

Such a T cell may use bona fide T cells as the starting material, for example, non-transformed T cells, terminally differentiated T cells, T cells having stable genome, and/or T cells that depend on cytokines and growth factors for proliferation and expansion. Alternatively, such a T cell may use T cells generated from precursor cells such as hematopoietic stem cells (e.g., iPSCs), e.g., in vitro culture. The T cells disclosed herein may confer one or more benefits in both CAR-T cell manufacturing and clinical applications.

In the Phase 1 CARBON trial, anti-CD19 CAR-T cells having a disrupted TRAC gene and a disrupted β2M gene as disclosed herein, and a nucleic acid encoding the anti-CD19 CAR as also disclosed herein, which is inserted at the disrupted TRAC gene, were safe and showed therapeutic efficacy in treating replapsed or refractory CD19+ B-cell malignancies who have received at least two prior lines of therapy. The patients enrolled in this Phase 1 trial include patients having the most aggressive disease presentations, including Diffuse Large B-cell Lymphoma (DLBCL) not otherwise specified (NOS), high-grade double- or triple-hit lymphomas, transformed follicular lymphoma, and grade 3B follicular lymphoma. Patients received the anti-CD19 CAR-T cells at doses ranging from Dose Level (DL) 1 (30 million CAR+ T cells) to DL4 (600 million CAR+ T cells), with an option to re-dose based on clinical benefit. Results from this clinica trial are provided below:
Data shows the potential for single infusions of the anti-CD19 CAR-T cells to achieve long-term durable complete remissions with a positively differentiated safety profile.
In a heavily pre-treated patient population with relapsed or refractory (R/R) LBCL (47% with ≥3 prior lines of therapy), the anti-CD19 CAR-T cells at DL≥3 (n=27) resulted in an ORR of 67% and CR rate of 41%.
Three patients remain in ongoing CR two years after treatment, and two additional patients remain in CR past one year.
No DLTs, no Graft versus Host Disease (GvHD) of any grade, and no Grade ≥3 cytokine release syndrome (CRS) events were observed Encouraging efficacy profile with several patients in ongoing CR beyond six months.
Clear evidence of the benefits of consolidation dosing (re-dosing) was observed, with deepening of CRs and conversions of stable disease and partial response to ongoing CRs after the second dose.
Safety profile remained consistent, confirming the tolerability of the consolidation regimen.
Peak expansion and overall pharmacokinetics of the anti-CD19 CAR-T cells were comparable between the initial and consolidation doses.

However, T cell exhaustion, occurring even prior to CAR T cell clearance by the immune system, can result in diminished efficacy and loss of response, especially in patients with high tumor burden.

In the instant disclosures, Regnase-1 (Reg1) and TGFBR2 (TGFBRII) have been added to gene edits such as TRAC (T-cell receptor to prevent GvHD), B2M (MHC class I to reduce T-cell mediated rejection), and optionally CD70 (to eliminate fratricide associated with anti-CD70 CAR-T cells and to increase potency), and site-specific CAR insertion at the TRAC locus using donor template such as an AAV template. Unexpectedly, the present disclosure reports that disruptions of Reg1 and TGFBRII synergistically acted to increase expansion and functional persistence of the anti-CD19 CAR-T cells and enhanced therapeutic efficacy against CD19+ cancers as observed in animal models. Disruption of Reg1 improves functional persistence by increasing cellular expansion potential, correlating with central memory phenotype. TGFBRII disruption allows CAR-T cells to avoid tumor microenvironment suppression. In combination, these edits increase potency at least 10-fold. In addition, long-term maintenance of Central memory T cells (T_{CM}) phenotype enables efficient manufacturing. Other advantageous features associated with the disruption of Reg1 gene and/or the TGFBRII gene may be found in WO2022064428, the relevant disclosures of which are incorporated by reference for the subject matter and purpose referenced herein.

The anti-CD19 CAR-T cell disclosed herein, having both Reg1 and TGFBRII genes knocked out, exhibited effective anti-tumor activities at low doses relative to anti-CD19 CAR-T cells with intact Reg1 and TGFBRII and long-term-persistence in vivo with robust CAR-T cell functionality. Further, it was observed that the anti-CD19 CAR-T cells disclosed here were effectively cleared in an immunocompetent mouse model. In sum, the anti-CD19 CAR-T cells disclosed herein, including the multiple gene edits also disclosed herein, have demonstrated increased potency, prolonged persistence, and sustained antitumor activity. The anti-CD19 CAR-T cells disclosed herein,

Accordingly, provided herein are anti-CD19 CAR-T cells having improved persistence and enhanced anti-tumor activity, methods of producing such T cells, and therapeutic applications of such T cells in eliminating CD19+ disease cells such as cancer cells. Such anti-CD19 CAR-T cells as disclosed herein, involving Reg1 and TGFBRII edits, have the potential to increase expansion and functional persistence, which may translate clinically to deeper, more durable responses. The anti-CD19 CAR-T cells provided herein therefore would be expected to show higher efficacy in treating target diseases such as relapsed and/or refractory B-cell malignancies.

### I. Anti-CD19 CAR-T Cells Having Enhanced Features

In some aspects, provided herein are anti-CD19 CAR-T cells with multiple genetic modifications to improve CAR-T cell functionality and thus therapeutic efficacy. The genetically engineered T cells provided herein express a chimeric antigen receptor (CAR) that binds CD19 and have multiple genetic edits on endogenous genes, for example, on a TRAC gene, a β2M gene, a Reg1 gene, and a TGFBRII gene.

The genetically engineered T cells may be derived from parent T cells (e.g., non-edited wild-type T cells) obtained from a suitable source, for example, one or more mammal donors. In some examples, the parent T cells are primary T cells (e.g., non-transformed and terminally differentiated T cells) obtained from one or more human donors. Alternatively, the parent T cells may be differentiated from precursor T cells obtained from one or more suitable donor or stem cells such as hematopoietic stem cells or inducible pluripotent stem cells (iPSC), which may be cultured in vitro.

Any of the genetically engineered T cells may be generated via gene editing (including genomic editing), a type of genetic engineering in which nucleotide(s)/nucleic acid(s) is/are inserted, deleted, and/or substituted in a DNA sequence, such as in the genome of a targeted cell. Targeted gene editing enables insertion, deletion, and/or substitution at pre-selected sites in the genome of a targeted cell (e.g., in a targeted gene or targeted DNA sequence). When a sequence of an endogenous gene is edited, for example by deletion, insertion or substitution of nucleotide(s)/nucleic acid(s), the endogenous gene comprising the affected sequence may be knocked-out due to the sequence alteration. Therefore, targeted editing may be used to disrupt endogenous gene expression. “Targeted integration” refers to a process involving insertion of one or more exogenous sequences, with or without deletion of an endogenous sequence at the insertion site. Targeted integration can result from targeted gene editing when a donor template containing an exogenous sequence is present.

A. Genetically Edited Genes

In some aspects, the present disclosure provides genetically engineered T cells that may comprise a disrupted Reg1 gene, a disrupted TGFBRII gene, a disrupted TRAC gene, and/or a disrupted β2M gene.

As used herein, a “disrupted gene” refers to a gene comprising an insertion, deletion or substitution relative to an endogenous gene such that expression of a functional protein from the endogenous gene is reduced or inhibited. As used herein, “disrupting a gene” refers to a method of inserting, deleting or substituting at least one nucleotide/nucleic acid in an endogenous gene such that expression of a functional protein from the endogenous gene is reduced or inhibited. Methods of disrupting a gene are known to those of skill in the art and described herein.

In some embodiments, a cell that comprises a disrupted gene does not express (e.g., at the cell surface) a detectable level (e.g., in an immune assay using an antibody binding to the encoded protein or by flow cytometry) of the protein encoded by the gene. A cell that does not express a detectable level of the protein may be referred to as a knockout cell.

Reg1 Gene Editing

In some embodiments, the genetically engineered T cells may comprise a disrupted gene involved in mRNA decay. Such a gene may be Reg1. Reg1 contains a zinc finger motif, binds RNA and exhibits ribonuclease activity. Reg1 plays roles in both immune and non-immune cells and its expression can be rapidly induced under diverse conditions including microbial infections, treatment with inflammatory cytokines and chemical or mechanical stimulation. Human Reg1 gene is located on chromosome 1p34.3. Additional information can be found in GenBank under Gene ID: 80149.

In some examples, the genetically engineered T cells may comprise a disrupted Reg1 gene such that the expression of Reg1 in the T cells is substantially reduced or eliminated completely. The disrupted Reg1 gene may comprise one or more genetic edits at one or more suitable target sites (e.g., in coding regions or in non-coding regulatory regions such as promoter regions) that disrupt expression of the Reg1 gene. Such target sites may be identified based on the gene editing approach for use in making the genetically engineered T cells. Exemplary target sites for the genetic edits may include exon 1, exon 2, exon 3, exon 4, exon 5, exon 6, or a combination thereof. In some examples, one or more genetic editing may occur in exon 2 or exon 4. Such genetic editing may be induced by the CRISPR/Cas technology using a suitable guide RNA, for example, those listed in Table 1. The resultant edited Reg1 gene using a gRNA listed in Table 1 may comprise one or more edited sequences provided in Table 4 below.

Disruption of the Reg1 gene can enhance long-term-persistence and maintain robust effector function, thereby improving T cell functionality.

TGFBRII Gene Editing

In some embodiments, the genetically engineered T cells may comprise a disrupted TGFBRII gene, which encodes Transforming Growth Factor Receptor Type II (TGFBRII). TGFBRII receptors are a family of serine/threonine kinase receptors involved in the TGFb signaling pathway. These receptors bind growth factor and cytokine signaling proteins in the TGFb family, for example, TGFβs (TGFβ1, TGFβ2, and TGFβ3), bone morphogenetic proteins (BMPs), growth differentiation factors (GDFs), activin and inhibin, myostatin, anti-Müllerian hormone (AMH), and NODAL.

In some examples, the genetically engineered T cells may comprise a disrupted TGFBRII gene such that the expression of TGFBRII in the T cells is substantially reduced or eliminated completely. The disrupted TGFBRII gene may comprise one or more genetic edits at one or more suitable target sites (e.g., in coding regions or in non-coding regulatory regions such as promoter regions) that disrupt expression of the TGFBRII gene. Such target sites may be identified based on the gene editing approach for use in making the genetically engineered T cells. Exemplary target sites for the genetic edits may include exon 1, exon 2, exon 3, exon 4, exon 5, or a combination thereof. In some examples, one or more genetic editing may occur in exon 4 and/or exon 5. Such genetic editing may be induced by a gene editing technology, (e.g., the CRISPR/Cas technology) using a suitable guide RNA, for example, those listed in Table 1. The resultant edited TGFBRII gene using a gRNA listed in Table 1 may comprise one or more edited sequences provided in Table 3 below.

Disruption of the TGFBRII gene can eliminate surface expression of TGFBRII and reduce the immunosuppressive effect of transforming growth factor beta (TFG-β) in the tumor microenvironment.

β2M Gene Edit

In some embodiments, the genetically engineered T cells disclosed herein may further comprise a disrupted β2M gene. β2M is a common (invariant) component of MHC I complexes. Disrupting its expression by gene editing will prevent host versus therapeutic allogeneic T cells responses leading to increased allogeneic T cell persistence. In some embodiments, expression of the endogenous β2M gene is eliminated to prevent a host-versus-graft response.

In some embodiments, an edited β2M gene may comprise a nucleotide sequence selected from the following sequences in Table 1. It is known to those skilled in the art that different nucleotide sequences in an edited gene such as an edited β2M gene (e.g., those in Table 2) may be generated by a single gRNA such as the one listed in Table 1 (β2M-1). See also WO2019097305, the relevant disclosures of which are incorporated by reference for the subject matter and purpose referenced herein.

TRAC Gene Edit

In some embodiments, the genetically engineered T cells as disclosed herein may further comprise a disrupted TRAC gene. This disruption leads to loss of function of the TCR and renders the engineered T cell non-alloreactive and suitable for allogeneic transplantation, minimizing the risk of graft versus host disease. In some embodiments, expression of the endogenous TRAC gene is eliminated to prevent a graft-versus-host response. See also WO2019097305, the relevant disclosures of which are incorporated by reference herein for the purpose and subject matter referenced herein.

Such genetic editing of the TRAC gene may be induced by a gene editing technology, (e.g., the CRISPR/Cas technology) using a suitable guide RNA, for example, those listed in Table 1. It should be understood that more than one suitable target site/gRNA can be used for each target gene disclosed herein, for example, those known in the art or disclosed herein. Additional examples can be found in, e.g., WO2019097305, the relevant disclosures of which are incorporated by reference herein for the purpose and subject matter referenced herein.

In some instances, a nucleic acid encoding an anti-CD19 CAR may be inserted into the TRAC gene, thereby disrupting expression of the TRAC gene. For example, the CAR-coding nucleic acid may replace the target site of a gRNA used in gene editing via CRISPR/Cas9 (e.g., replacing the fragment comprising SEQ ID NO: 18 in the TRAC gene.

B. Anti-CD19 Chimeric Antigen Receptor (CAR)

A chimeric antigen receptor (CAR) refers to an artificial immune cell receptor that is engineered to recognize and bind to an antigen expressed by undesired cells, for example, disease cells such as cancer cells. A T cell that expresses a CAR polypeptide is referred to as a CAR T cell. CARs have the ability to redirect T-cell specificity and reactivity toward a selected target in a non-MHC-restricted manner. The non-MHC-restricted antigen recognition gives CAR-T cells the ability to recognize an antigen independent of antigen processing, thus bypassing a major mechanism of tumor escape. Moreover, when expressed on T-cells, CARs advantageously do not dimerize with endogenous T-cell receptor (TCR) alpha and beta chains.

There are various generations of CARs, each of which contains different components. First generation CARs join an antibody-derived scFv to the CD3zeta (ζ or z) intracellular signaling domain of the T-cell receptor through hinge and transmembrane domains. Second generation CARs incorporate an additional co-stimulatory domain, e.g., CD28, 4-1BB (41BB), or ICOS, to supply a costimulatory signal. Third-generation CARs contain two costimulatory domains (e.g., a combination of CD27, CD28, 4-1BB, ICOS, or OX40) fused with the TCR CD3ζ chain. Maude et al., Blood. 2015; 125(26):4017-4023; Kakarla and Gottschalk, Cancer J. 2014; 20(2):151-155). Any of the various generations of CAR constructs is within the scope of the present disclosure.

Generally, a CAR is a fusion polypeptide comprising an extracellular domain that recognizes a target antigen (e.g., a single chain fragment (scFv) of an antibody or other antibody fragment) and an intracellular domain comprising a signaling domain of the T-cell receptor (TCR) complex (e.g., CD3ζ) and, in most cases, a co-stimulatory domain. (Enblad et al., Human Gene Therapy. 2015; 26(8):498-505). A CAR construct may further comprise a hinge and transmembrane domain between the extracellular domain and the intracellular domain, as well as a signal peptide at the N-terminus for surface expression. Examples of signal peptides include SEQ ID NO: 51 and SEQ ID NO: 52 as provided in Table 5 below. Other signal peptides may be used.

(i) Antigen Binding Extracellular Domain

The antigen-binding extracellular domain is the region of a CAR polypeptide that is exposed to the extracellular fluid when the CAR is expressed on cell surface. In some instances, a signal peptide may be located at the N-terminus to facilitate cell surface expression. In some embodiments, the antigen binding domain can be a single-chain variable fragment (scFv, which may include an antibody heavy chain variable region (V_{H}) and an antibody light chain variable region (VL) (in either orientation). In some instances, the VH and VL fragment may be linked via a peptide linker. The linker, in some embodiments, includes hydrophilic residues with stretches of glycine and serine for flexibility as well as stretches of glutamate and lysine for added solubility. The scFv fragment retains the antigen-binding specificity of the parent antibody, from which the scFv fragment is derived. In some embodiments, the scFv may comprise humanized VH and/or VL domains. In other embodiments, the VH and/or VL domains of the scFv are fully human.

The antigen-binding extracellular domain may be specific to a CD19 antigen, such as a human CD19 antigen.

In some embodiments, the antigen-binding extracellular domain can be a single-chain variable fragment (scFv) that binds CD19 as disclosed herein. The scFv may comprise an antibody heavy chain variable region (V_{H}) and an antibody light chain variable region (V_{L}), which optionally may be connected via a flexible peptide linker. In some instances, the scFv may have the V_{H} to V_{L} orientation (from N-terminus to C-terminus). Alternatively, the scFv may have the V_{L} to V_{H} orientation (from N-terminus to C-terminus).

In some examples, the antigen-binding extracellular domain can be a single-chain variable fragment (scFv) that binds human CD19. In some instances, the anti-CD19 scFv may comprises (i) a heavy chain variable region (V_{H}) that comprises the same heavy chain complementary determining regions (CDRs) as those in SEQ ID NO: 81; and (ii) a light chain variable region (V_{L}) that comprises the same light chain CDRs as those in SEQ ID NO: 82. See Table 5 below. In some specific examples, the anti-CD19 antibody discloses herein may comprise the heavy chain CDR1, heavy chain CDR2, and heavy chain CDR3 set forth as SEQ ID NOs: 63-65, respectively as determined by the Kabat method. Alternatively or in addition, the anti-CD19 antibody discloses herein may comprise the light chain CDR1, light chain CDR2, and light chain CDR3 set forth as SEQ ID NOs:60-62 as determined by the Kabat method. Alternatively, the anti-CD19 antibody discloses herein may comprise the heavy chain CDR1, heavy chain CDR2, and heavy chain CDR3 set forth as SEQ ID NOs: 69-71, respectively as determined by the Chothia method. Alternatively or in addition, the anti-CD19 antibody discloses herein may comprise the light chain CDR1, light chain CDR2, and light chain CDR3 set forth as SEQ ID NOs:66-68 as determined by the Chothia method. In one specific example, the anti-CD19 scFv may comprise a V_{H} comprising the amino acid sequence of SEQ ID NO: 81 and a V_{L} comprises the amino acid sequence of SEQ ID NO: 82. See Sequence Table 5 below.

Two antibodies having the same V_{H} and/or V_{L} CDRs means that their CDRs are identical when determined by the same approach (e.g., the Kabat approach, the Chothia approach, the AbM approach, the Contact approach, or the IMGT approach as known in the art. See, e.g., bioinf.org.uk/abs/ or abysis.org/abysis/sequence_input).

(ii) Transmembrane Domain

The anti-CD19 CAR polypeptide disclosed herein may contain a transmembrane domain, which can be a hydrophobic alpha helix that spans the membrane. As used herein, a “transmembrane domain” refers to any protein structure that is thermodynamically stable in a cell membrane, preferably a eukaryotic cell membrane. The transmembrane domain can provide stability of the anti-CD19 CAR containing such.

In some embodiments, the transmembrane domain of a anti-CD19 CAR as provided herein can be a CD8 transmembrane domain. In other embodiments, the transmembrane domain can be a CD28 transmembrane domain. In yet other embodiments, the transmembrane domain is a chimera of a CD8 and CD28 transmembrane domain. Other transmembrane domains may be used as provided herein. In some embodiments, the transmembrane domain is a CD8a transmembrane domain containing the sequence of SEQ ID NO: 53 as provided below in Table 5. Other transmembrane domains may be used.

(iii) Hinge Domain

In some embodiments, a hinge domain may be located between an extracellular domain (comprising the antigen binding domain) and a transmembrane domain of an anti-CD19 CAR, or between a cytoplasmic domain and a transmembrane domain of the anti-CD19 CAR. A hinge domain can be any oligopeptide or polypeptide that functions to link the transmembrane domain to the extracellular domain and/or the cytoplasmic domain in the polypeptide chain. A hinge domain may function to provide flexibility to the anti-CD19 CAR, or domains thereof, or to prevent steric hindrance of the anti-CD19 CAR, or domains thereof.

In some embodiments, a hinge domain may comprise up to 300 amino acids (e.g., 10 to 100 amino acids, or 5 to 20 amino acids). In some embodiments, one or more hinge domain(s) may be included in other regions of a anti-CD19 CAR. In some embodiments, the hinge domain may be a CD8 hinge domain. Other hinge domains may be used.

(iv) Intracellular Signaling Domains

Any of the anti-CD19 CAR constructs contain one or more intracellular signaling domains (e.g., CD3ζ, and optionally one or more co-stimulatory domains), which are the functional end of the receptor. Following antigen recognition, receptors cluster and a signal is transmitted to the cell.

CD3ζ is the cytoplasmic signaling domain of the T cell receptor complex. CD3ζ contains three (3) immunoreceptor tyrosine-based activation motif (ITAM)s, which transmit an activation signal to the T cell after the T cell is engaged with a cognate antigen. In many cases, CD3ζ provides a primary T cell activation signal but not a fully competent activation signal, which requires a co-stimulatory signaling.

In some embodiments, the anti-CD19 CAR polypeptides disclosed herein may further comprise one or more co-stimulatory signaling domains. For example, the co-stimulatory domains of CD28 and/or 4-1BB may be used to transmit a full proliferative/survival signal, together with the primary signaling mediated by CD3ζ. In some examples, the CAR disclosed herein comprises a CD28 co-stimulatory molecule. In other examples, the CAR disclosed herein comprises a 4-1BB co-stimulatory molecule. In some embodiments, a CAR includes a CD3ζ signaling domain and a CD28 co-stimulatory domain. In other embodiments, a CAR includes a CD3ζ signaling domain and 4-1BB co-stimulatory domain. In still other embodiments, a CAR includes a CD3ζ signaling domain, a CD28 co-stimulatory domain, and a 4-1BB co-stimulatory domain.

Table 5 provides examples of signaling domains derived from 4-1BB, CD28 and CD3-zeta that may be used herein.

In specific examples, the anti-CD19 CAR disclosed herein may comprise the amino acid sequence of SEQ ID NO: 73, which may be encoded by the nucleotide sequence of SEQ ID NO: 72. Alternatively, the anti-CD19 CAR may be a mature form without the N-terminal signal peptide, e.g., comprising the amino acid sequence of SEQ ID NO:74.

C. Methods of Making Genetically Engineered T Cells

The genetically engineered T cells disclosed herein can be prepared by genetic editing of parent T cells or precursor cells thereof via a conventional gene editing method or those described herein.

(a) T cells

In some embodiments, T cells can be derived from one or more suitable mammals, for example, one or more human donors. T cells can be obtained from a number of sources, including, but not limited to, peripheral blood mononuclear cells, bone marrow, lymph nodes tissue, cord blood, thymus issue, tissue from a site of infection, ascites, pleural effusion, spleen tissue, and tumors. In certain embodiments, T cells can be obtained from a unit of blood collected from a subject using any number of techniques known to the skilled person, such as sedimentation, e.g., FICOLL™ separation.

In some examples, T cells can be isolated from a mixture of immune cells (e.g., those described herein) to produce an isolated T cell population. For example, after isolation of peripheral blood mononuclear cells (PBMC), both cytotoxic and helper T lymphocytes can be sorted into naive, memory, and effector T cell subpopulations either before or after activation, expansion, and/or genetic modification.

A specific subpopulation of T cells, expressing one or more of the following cell surface markers: TCRab, CD3, CD4, CD8, CD27 CD28, CD38 CD45RA, CD45RO, CD62L, CD127, CD122, CD95, CD197, CCR7, KLRG1, MCH-I proteins and/or MCH-II proteins, can be further isolated by positive or negative selection techniques. In some embodiments, a specific subpopulation of T cells, expressing one or more of the markers selected from the group consisting of TCRab, CD4 and/or CD8, is further isolated by positive or negative selection techniques. In some embodiments, subpopulations of T cells may be isolated by positive or negative selection prior to genetic engineering and/or post genetic engineering.

An isolated population of T cells may express one or more of the T cell markers, including, but not limited to a CD3+, CD4+, CD8+, or a combination thereof. In some embodiments, the T cells are isolated from a donor, or subject, and first activated and stimulated to proliferate in vitro prior to undergoing gene editing.

In some instances, the T cell population comprises primary T cells isolated from one or more human donors. Such T cells are terminally differentiated, not transformed, depend on cytokines and/or growth factors for growth, and/or have stable genomes.

Alternatively, the T cells may be derived from stem cells (e.g., HSCs or iPSCs) via in vitro differentiation.

T cells from a suitable source can be subjected to one or more rounds of stimulation, activation and/or expansion. T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041. In some embodiments, T cells can be activated and expanded for about 1 day to about 4 days, about 1 day to about 3 days, about 1 day to about 2 days, about 2 days to about 3 days, about 2 days to about 4 days, about 3 days to about 4 days, or about 1 day, about 2 days, about 3 days, or about 4 days prior to introduction of the genome editing compositions into the T cells.

In some embodiments, T cells are activated and expanded for about 4 hours, about 6 hours, about 12 hours, about 18 hours, about 24 hours, about 36 hours, about 48 hours, about 60 hours, or about 72 hours prior to introduction of the gene editing compositions into the T cells. In some embodiments, T cells are activated at the same time that genome editing compositions are introduced into the T cells. In some instances, the T cell population can be expanded and/or activated after the genetic editing as disclosed herein. T cell populations or isolated T cells generated by any of the gene editing methods described herein are also within the scope of the present disclosure.

(b) Gene Editing Methods

Any of the genetically engineered T cells can be prepared using conventional gene editing methods or those described herein to edit one or more of the target genes disclosed herein (targeted editing). Targeted editing can be achieved either through a nuclease-independent approach, or through a nuclease-dependent approach. In the nuclease-independent targeted editing approach, homologous recombination is guided by homologous sequences flanking an exogenous polynucleotide to be introduced into an endogenous sequence through the enzymatic machinery of the host cell. The exogenous polynucleotide may introduce deletions, insertions or replacement of nucleotides in the endogenous sequence.

Alternatively, the nuclease-dependent approach can achieve targeted editing with higher frequency through the specific introduction of double strand breaks (DSBs) by specific rare-cutting nucleases (e.g., endonucleases). Such nuclease-dependent targeted editing also utilizes DNA repair mechanisms, for example, non-homologous end joining (NHEJ), which occurs in response to DSBs. DNA repair by NHEJ often leads to random insertions or deletions (indels) of a small number of endogenous nucleotides. In contrast to NHEJ mediated repair, repair can also occur by a homology directed repair (HDR). When a donor template containing exogenous genetic material flanked by a pair of homology arms is present, the exogenous genetic material can be introduced into the genome by HDR, which results in targeted integration of the exogenous genetic material.

In some embodiments, gene disruption may occur by deletion of a genomic sequence using two guide RNAs. Methods of using CRISPR-Cas gene editing technology to create a genomic deletion in a cell (e.g., to knock out a gene in a cell) are known (Bauer D E et al. Vis. Exp. 2015; 95:e52118).

Available endonucleases capable of introducing specific and targeted DSB s include, but not limited to, zinc-finger nucleases (ZFN), transcription activator-like effector nucleases (TALEN), and RNA-guided CRISPR-Cas9 nuclease (CRISPR/Cas9; Clustered Regular Interspaced Short Palindromic Repeats Associated 9). Additionally, DICE (dual integrase cassette exchange) system utilizing phiC31 and Bxb1 integrases may also be used for targeted integration. Some exemplary approaches are disclosed in detail below.

CRISPR-Cas9 Gene Editing System

The CRISPR-Cas9 system is a naturally-occurring defense mechanism in prokaryotes that has been repurposed as an RNA-guided DNA-targeting platform used for gene editing. It relies on the DNA nuclease Cas9, and two noncoding RNAs, crisprRNA (crRNA) and trans-activating RNA (tracrRNA), to target the cleavage of DNA. CRISPR is an abbreviation for Clustered Regularly Interspaced Short Palindromic Repeats, a family of DNA sequences found in the genomes of bacteria and archaea that contain fragments of DNA (spacer DNA) with similarity to foreign DNA previously exposed to the cell, for example, by viruses that have infected or attacked the prokaryote. These fragments of DNA are used by the prokaryote to detect and destroy similar foreign DNA upon re-introduction, for example, from similar viruses during subsequent attacks. Transcription of the CRISPR locus results in the formation of an RNA molecule comprising the spacer sequence, which associates with and targets Cas (CRISPR-associated) proteins able to recognize and cut the foreign, exogenous DNA. Numerous types and classes of CRISPR/Cas systems have been described (see, e.g., Koonin et al., (2017) Curr Opin Microbiol 37:67-78).

crRNA drives sequence recognition and specificity of the CRISPR-Cas9 complex through Watson-Crick base pairing typically with a 20 nucleotide (nt) sequence in the target DNA. Changing the sequence of the 5′ 20nt in the crRNA allows targeting of the CRISPR-Cas9 complex to specific loci. The CRISPR-Cas9 complex only binds DNA sequences that contain a sequence match to the first 20 nt of the crRNA, if the target sequence is followed by a specific short DNA motif (with the sequence NGG) referred to as a protospacer adjacent motif (PAM).

tracrRNA hybridizes with the 3′ end of crRNA to form an RNA-duplex structure that is bound by the Cas9 endonuclease to form the catalytically active CRISPR-Cas9 complex, which can then cleave the target DNA.

Once the CRISPR-Cas9 complex is bound to DNA at a target site, two independent nuclease domains within the Cas9 enzyme each cleave one of the DNA strands upstream of the PAM site, leaving a double-strand break (DSB) where both strands of the DNA terminate in a base pair (a blunt end).

After binding of CRISPR-Cas9 complex to DNA at a specific target site and formation of the site-specific DSB, the next key step is repair of the DSB. Cells use two main DNA repair pathways to repair the DSB: non-homologous end joining (NHEJ) and homology-directed repair (HDR).

NHEJ is a robust repair mechanism that appears highly active in the majority of cell types, including non-dividing cells. NHEJ is error-prone and can often result in the removal or addition of between one and several hundred nucleotides at the site of the DSB, though such modifications are typically <20 nt. The resulting insertions and deletions (indels) can disrupt coding or noncoding regions of genes. Alternatively, HDR uses a long stretch of homologous donor DNA, provided endogenously or exogenously, to repair the DSB with high fidelity. HDR is active only in dividing cells, and occurs at a relatively low frequency in most cell types. In many embodiments of the present disclosure, NHEJ is utilized as the repair operant.

Endonuclease for Use in CRISPR

In some embodiments, the Cas9 (CRISPR associated protein 9) endonuclease is used in a CRISPR method for making the genetically engineered T cells as disclosed herein. The Cas9 enzyme may be one from Streptococcus pyogenes, although other Cas9 homologs may also be used. It should be understood, that wild-type Cas9 may be used or modified versions of Cas9 may be used (e.g., evolved versions of Cas9, or Cas9 orthologues or variants), as provided herein. In some embodiments, Cas9 may be substituted with another RNA-guided endonuclease, such as Cpf1 (of a class II CRISPR/Cas system).

In some embodiments, the CRISPR/Cas system comprises components derived from a Type-I, Type-II, or Type-III system. Updated classification schemes for CRISPR/Cas loci define Class 1 and Class 2 CRISPR/Cas systems, having Types I to V or VI (Makarova et al., (2015) Nat Rev Microbiol, 13(11):722-36; Shmakov et al., (2015) Mol Cell, 60:385-397). Class 2 CRISPR/Cas systems have single protein effectors. Cas proteins of Types II, V, and VI are single-protein, RNA-guided endonucleases, herein called “Class 2 Cas nucleases.” Class 2 Cas nucleases include, for example, Cas9, Cpf1, C2c1, C2c2, and C2c3 proteins. The Cpf1 nuclease (Zetsche et al., (2015) Cell 163:1-13) is homologous to Cas9 and contains a RuvC-like nuclease domain.

In some embodiments, the Cas nuclease is from a Type-II CRISPR/Cas system (e.g., a Cas9 protein from a CRISPR/Cas9 system). In some embodiments, the Cas nuclease is from a Class 2 CRISPR/Cas system (a single-protein Cas nuclease such as a Cas9 protein or a Cpf1 protein). The Cas9 and Cpf1 family of proteins are enzymes with DNA endonuclease activity, and they can be directed to cleave a desired nucleic acid target by designing an appropriate guide RNA, as described further herein.

In some embodiments, a Cas nuclease may comprise more than one nuclease domain. For example, a Cas9 nuclease may comprise at least one RuvC-like nuclease domain (e.g., Cpf1) and at least one HNH-like nuclease domain (e.g., Cas9). In some embodiments, the Cas9 nuclease introduces a DSB in the target sequence. In some embodiments, the Cas9 nuclease is modified to contain only one functional nuclease domain. For example, the Cas9 nuclease is modified such that one of the nuclease domains is mutated or fully or partially deleted to reduce its nucleic acid cleavage activity. In some embodiments, the Cas9 nuclease is modified to contain no functional RuvC-like nuclease domain. In other embodiments, the Cas9 nuclease is modified to contain no functional HNH-like nuclease domain. In some embodiments in which only one of the nuclease domains is functional, the Cas9 nuclease is a nickase that is capable of introducing a single-stranded break (a “nick”) into the target sequence. In some embodiments, a conserved amino acid within a Cas9 nuclease domain is substituted to reduce or alter a nuclease activity. In some embodiments, the Cas nuclease nickase comprises an amino acid substitution in the RuvC-like nuclease domain. Exemplary amino acid substitutions in the RuvC-like nuclease domain include D10A (based on the S. pyogenes Cas9 nuclease). In some embodiments, the nickase comprises an amino acid substitution in the HNH-like nuclease domain. Exemplary amino acid substitutions in the HNH-like nuclease domain include E762A, H840A, N863A, H983A, and D986A (based on the S. pyogenes Cas9 nuclease). One example is provided in Table 1 below (SEQ ID NO: 29).

In some embodiments, the Cas nuclease is from a Type-I CRISPR/Cas system. In some embodiments, the Cas nuclease is a component of the Cascade complex of a Type-I CRISPR/Cas system. For example, the Cas nuclease is a Cas3 nuclease. In some embodiments, the Cas nuclease is derived from a Type-III CRISPR/Cas system. In some embodiments, the Cas nuclease is derived from Type-IV CRISPR/Cas system. In some embodiments, the Cas nuclease is derived from a Type-V CRISPR/Cas system. In some embodiments, the Cas nuclease is derived from a Type-VI CRISPR/Cas system.

Guide RNAs (gRNAs)

The CRISPR technology involves the use of a genome-targeting nucleic acid that can direct the endonuclease to a specific target sequence within a target gene for gene editing at the specific target sequence. The genome-targeting nucleic acid can be a RNA. A genome-targeting RNA is referred to as a “guide RNA” or “gRNA” herein. A guide RNA comprises at least a spacer sequence that hybridizes to a target nucleic acid sequence within a target gene for editing, and a CRISPR repeat sequence.

In Type II systems, the gRNA also comprises a second RNA called the tracrRNA sequence. In the Type II gRNA, the CRISPR repeat sequence and tracrRNA sequence hybridize to each other to form a duplex. In the Type V gRNA, the crRNA forms a duplex. In both systems, the duplex binds a site-directed polypeptide, such that the guide RNA and site-direct polypeptide form a complex. In some embodiments, the genome-targeting nucleic acid provides target specificity to the complex by virtue of its association with the site-directed polypeptide. The genome-targeting nucleic acid thus directs the activity of the site-directed polypeptide.

As is understood by the person of ordinary skill in the art, each guide RNA is designed to include a spacer sequence complementary to its genomic target sequence. See Jinek et al., Science, 337, 816-821 (2012) and Deltcheva et al., Nature, 471, 602-607 (2011).

In some embodiments, the genome-targeting nucleic acid (e.g., gRNA) is a double-molecule guide RNA. In some embodiments, the genome-targeting nucleic acid (e.g., gRNA) is a single-molecule guide RNA.

A double-molecule guide RNA comprises two strands of RNA molecules. The first strand comprises in the 5′ to 3′ direction, an optional spacer extension sequence, a spacer sequence and a minimum CRISPR repeat sequence. The second strand comprises a minimum tracrRNA sequence (complementary to the minimum CRISPR repeat sequence), a 3′ tracrRNA sequence and an optional tracrRNA extension sequence.

A single-molecule guide RNA (referred to as a “sgRNA”) in a Type II system comprises, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may comprise elements that contribute additional functionality (e.g., stability) to the guide RNA. The single-molecule guide linker links the minimum CRISPR repeat and the minimum tracrRNA sequence to form a hairpin structure. The optional tracrRNA extension comprises one or more hairpins. A single-molecule guide RNA in a Type V system comprises, in the 5′ to 3′ direction, a minimum CRISPR repeat sequence and a spacer sequence.

A spacer sequence in a gRNA is a sequence (e.g., a 20-nucleotide sequence) that defines the target sequence (e.g., a DNA target sequences, such as a genomic target sequence) of a target gene of interest. In some embodiments, the spacer sequence ranges from 15 to 30 nucleotides. For example, the spacer sequence may contain 15, 16, 17, 18, 19, 29, 21, 22, 23, 24, 25, 26, 27, 28, 29, or 30 nucleotides. In some embodiments, a spacer sequence contains 20 nucleotides.

The “target sequence” is in a target gene that is adjacent to a PAM sequence and is the sequence to be modified by an RNA-guided nuclease (e.g., Cas9). The “target sequence” is on the so-called PAM-strand in a “target nucleic acid,” which is a double-stranded molecule containing the PAM-strand and a complementary non-PAM strand. One of skill in the art recognizes that the gRNA spacer sequence hybridizes to the complementary sequence located in the non-PAM strand of the target nucleic acid of interest. Thus, the gRNA spacer sequence is the RNA equivalent of the target sequence. For example, if the target sequence is 5′-AGAGCAACAGTGCTGTGGCC**-3′ (SEQ ID NO: 18), then the gRNA spacer sequence is 5′-AGAGCAACAGUGCUGUGGCC**-3′ (SEQ ID NO: 15). The spacer of a gRNA interacts with a target nucleic acid of interest in a sequence-specific manner via hybridization (i.e., base pairing). The nucleotide sequence of the spacer thus varies depending on the target sequence of the target nucleic acid of interest.

In a CRISPR/Cas system herein, the spacer sequence is designed to hybridize to a region of the target nucleic acid that is located 5′ of a PAM recognizable by a Cas9 enzyme used in the system. The spacer may perfectly match the target sequence or may have mismatches. Each Cas9 enzyme has a particular PAM sequence that it recognizes in a target DNA. For example, S. pyogenes recognizes in a target nucleic acid a PAM that comprises the sequence 5′-NRG-3′, where R comprises either A or G, where N is any nucleotide and N is immediately 3′ of the target nucleic acid sequence targeted by the spacer sequence.

In some embodiments, the target nucleic acid sequence has 20 nucleotides in length. In some embodiments, the target nucleic acid has less than 20 nucleotides in length. In some embodiments, the target nucleic acid has more than 20 nucleotides in length. In some embodiments, the target nucleic acid has at least: 5, 10, 15, 16, 17, 18, 19, 20, 21, 22, 23, 24, 25, 30 or more nucleotides in length. In some embodiments, the target nucleic acid has at most: 5, 10, 15, 16, 17, 18, 19, 20, 21, 22, 23, 24, 25, 30 or more nucleotides in length. In some embodiments, the target nucleic acid sequence has 20 bases immediately 5′ of the first nucleotide of the PAM. For example, in a sequence comprising 5′-NNNNNNNNNNNNNNNNNNNNNRG-3′, the target nucleic acid can be the sequence that corresponds to the Ns, wherein N can be any nucleotide, and the underlined NRG sequence is the S. pyogenes PAM.

The guide RNA disclosed herein may target any sequence of interest via the spacer sequence in the crRNA. In some embodiments, the degree of complementarity between the spacer sequence of the guide RNA and the target sequence in the target gene can be about 60%, 65%, 70%, 75%, 80%, 85%, 90%, 95%, 97%, 98%, 99%, or 100%. In some embodiments, the spacer sequence of the guide RNA and the target sequence in the target gene is 100% complementary. In other embodiments, the spacer sequence of the guide RNA and the target sequence in the target gene may contain up to 10 mismatches, e.g., up to 9, up to 8, up to 7, up to 6, up to 5, up to 4, up to 3, up to 2, or up to 1 mismatch.

For any of the gRNA sequences provided herein, those that do not explicitly indicate modifications are meant to encompass both unmodified sequences and sequences having any suitable modifications.

The length of the spacer sequence in any of the gRNAs disclosed herein may depend on the CRISPR/Cas9 system and components used for editing any of the target genes also disclosed herein. For example, different Cas9 proteins from different bacterial species have varying optimal spacer sequence lengths. Accordingly, the spacer sequence may have 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the spacer sequence may have 18-24 nucleotides in length. In some embodiments, the targeting sequence may have 19-21 nucleotides in length. In some embodiments, the spacer sequence may comprise 20 nucleotides in length.

In some embodiments, the gRNA can be an sgRNA, which may comprise a 20 nucleotides spacer sequence at the 5′ end of the sgRNA sequence. In some embodiments, the sgRNA may comprise a less than 20 nucleotide spacer sequence at the 5′ end of the sgRNA sequence. In some embodiments, the sgRNA may comprise a more than 20 nucleotide spacer sequence at the 5′ end of the sgRNA sequence. In some embodiments, the sgRNA comprises a variable length spacer sequence with 17-30 nucleotides at the 5′ end of the sgRNA sequence. Examples are provided in Table 1 below. In these exemplary sequences, the fragment of “n” refers to the spacer sequence at the 5′ end.

In some embodiments, the sgRNA comprises comprise no uracil at the 3′ end of the sgRNA sequence. In other embodiments, the sgRNA may comprise one or more uracil at the 3′ end of the sgRNA sequence. For example, the sgRNA can comprise 1-8 uracil residues, at the 3′ end of the sgRNA sequence, e.g., 1, 2, 3, 4, 5, 6, 7, or 8 uracil residues at the 3′ end of the sgRNA sequence.

Any of the gRNAs disclosed herein, including any of the sgRNAs, may be unmodified. Alternatively, it may contain one or more modified nucleotides and/or modified backbones. For example, a modified gRNA such as an sgRNA can comprise one or more 2′-O-methyl phosphorothioate nucleotides, which may be located at either the 5′ end, the 3′ end, or both.

In certain embodiments, more than one guide RNAs can be used with a CRISPR/Cas nuclease system. Each guide RNA may contain a different targeting sequence, such that the CRISPR/Cas system cleaves more than one target nucleic acid. In some embodiments, one or more guide RNAs may have the same or differing properties such as activity or stability within the Cas9 RNP complex. Where more than one guide RNA is used, each guide RNA can be encoded on the same or on different vectors. The promoters used to drive expression of the more than one guide RNA is the same or different.

In some embodiments, the gRNAs disclosed herein target a Reg1 gene, for example, target a site within exon 1, exon 2, exon 3, exon 4, exon 5, or exon 6 of the Reg1 gene. Such a gRNA may comprise a spacer sequence complementary (complete or partially) to the target sequences in exon 2 or exon 4 of a Reg1 gene, or a fragment thereof. Exemplary target sequences of Reg1 and exemplary gRNA sequences are provided in Table 1 below.

In some embodiments, the gRNAs disclosed herein target a TGFBRII gene, for example, target a site within exon 1, exon 2, exon 3, exon 4, exon 5, or exon 6 of the TGFBRII gene. Such a gRNA may comprise a spacer sequence complementary (complete or partially) to the target sequences in exon 4 or exon 5 of a TGFBRII gene, or a fragment thereof. Exemplary target sequences of TGFBRII and exemplary gRNA sequences are provided in Table 1 below.

In some embodiments, the gRNAs disclosed herein target a β2M gene, for example, target a suitable site within a β2M gene. See also WO2019097305, the relevant disclosures of which are incorporated by reference herein for the purpose and subject matter referenced herein. Other gRNA sequences may be designed using the β2M gene sequence located on Chromosome 15 (GRCh38 coordinates: Chromosome 15: 44,711,477-44,718,877; Ensembl: ENSG00000166710). In some embodiments, gRNAs targeting the β2M genomic region and RNA-guided nuclease create breaks in the β2M genomic region resulting in Indels in the β2M gene disrupting expression of the mRNA or protein.

In some embodiments, the gRNAs disclosed herein target a TRAC gene. See also WO2019097305, the relevant disclosures of which are incorporated by reference herein for the subject matter and purpose referenced herein. Other gRNA sequences may be designed using the TRAC gene sequence located on chromosome 14 (GRCh38: chromosome 14: 22,547,506-22,552,154. Ensembl; ENSG00000277734). In some embodiments, gRNAs targeting the TRAC genomic region and RNA-guided nuclease create breaks in the TRAC genomic region resulting Indels in the TRAC gene disrupting expression of the mRNA or protein.

Exemplary spacer sequences and gRNAs targeting a β2M gene or TRAC gene are provided in Table 1 below.

By way of illustration, guide RNAs used in the CRISPR/Cas/Cpf1 system, or other smaller RNAs can be readily synthesized by chemical means, as illustrated below and described in the art. While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together. Much longer RNAs, such as those encoding a Cas9 or Cpf1 endonuclease, are more readily generated enzymatically. Various types of RNA modifications can be introduced during or after chemical synthesis and/or enzymatic generation of RNAs, e.g., modifications that enhance stability, reduce the likelihood or degree of innate immune response, and/or enhance other attributes, as described in the art.

In some examples, the gRNAs of the present disclosure can be produced in vitro transcription (IVT), synthetic and/or chemical synthesis methods, or a combination thereof. Enzymatic (IVT), solid-phase, liquid-phase, combined synthetic methods, small region synthesis, and ligation methods are utilized. In one embodiment, the gRNAs are made using IVT enzymatic synthesis methods. Methods of making polynucleotides by IVT are known in the art and are described in WO2013/151666. Accordingly, the present disclosure also includes polynucleotides, e.g., DNA, constructs and vectors are used to in vitro transcribe a gRNA described herein.

Various types of RNA modifications can be introduced during or after chemical synthesis and/or enzymatic generation of RNAs, e.g., modifications that enhance stability, reduce the likelihood or degree of innate immune response, and/or enhance other attributes, as described in the art. In some embodiments, non-natural modified nucleobases can be introduced into any of the gRNAs disclosed herein during synthesis or post-synthesis. In certain embodiments, modifications are on internucleoside linkages, purine or pyrimidine bases, or sugar. In some embodiments, a modification is introduced at the terminal of a gRNA with chemical synthesis or with a polymerase enzyme. Examples of modified nucleic acids and their synthesis are disclosed in WO2013/052523. Synthesis of modified polynucleotides is also described in Verma and Eckstein, Annual Review of Biochemistry, vol. 76, 99-134 (1998).

In some embodiments, enzymatic or chemical ligation methods can be used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1(3), 165-187 (1990).

In some embodiments of the present disclosure, a CRISPR/Cas nuclease system for use in genetically editing any of the target genes disclosed here may include at least one guide RNA. In some examples, the CRISPR/Cas nuclease system may contain multiple gRNAs, for example, 2, 3, or 4 gRNAs. Such multiple gRNAs may target different sites in a same target gene. Alternatively, the multiple gRNAs may target different genes. In some embodiments, the guide RNA(s) and the Cas protein may form a ribonucleoprotein (RNP), e.g., a CRISPR/Cas complex. The guide RNA(s) may guide the Cas protein to a target sequence(s) on one or more target genes as those disclosed herein, where the Cas protein cleaves the target gene at the target site. In some embodiments, the CRISPR/Cas complex is a Cpf1/guide RNA complex. In some embodiments, the CRISPR complex is a Type-II CRISPR/Cas9 complex. In some embodiments, the Cas protein is a Cas9 protein. In some embodiments, the CRISPR/Cas9 complex is a Cas9/guide RNA complex.

In some embodiments, the indel frequency (editing frequency) of a particular CRISPR/Cas nuclease system, comprising one or more specific gRNAs, may be determined using a TIDE analysis, which can be used to identify highly efficient gRNA molecules for editing a target gene. In some embodiments, a highly efficient gRNA yields a gene editing frequency of higher than 80%. For example, a gRNA is considered to be highly efficient if it yields a gene editing frequency of at least 80%, at least 85%, at least 90%, at least 95%, or 100%.

Delivery of Guide RNAs and Nucleases to T Cells

The CRISPR/Cas nuclease system disclosed herein, comprising one or more gRNAs and at least one RNA-guided nuclease, optionally a donor template as disclosed below, can be delivered to a target cell (e.g., a T cell) for genetic editing of a target gene, via a conventional method. In some embodiments, components of a CRISPR/Cas nuclease system as disclosed herein may be delivered to a target cell separately, either simultaneously or sequentially. In other embodiments, the components of the CRISPR/Cas nuclease system may be delivered into a target together, for example, as a complex. In some instances, gRNA and a RNA-guided nuclease can be pre-complexed together to form a ribonucleoprotein (RNP), which can be delivered into a target cell.

RNPs are useful for gene editing, at least because they minimize the risk of promiscuous interactions in a nucleic acid-rich cellular environment and protect the RNA from degradation. Methods for forming RNPs are known in the art. In some embodiments, an RNP containing an RNA-guided nuclease (e.g., a Cas nuclease, such as a Cas9 nuclease) and one or more gRNAs targeting one or more genes of interest can be delivered a cell (e.g., a T cell). In some embodiments, an RNP can be delivered to a T cell by electroporation.

In some embodiments, an RNA-guided nuclease can be delivered to a cell in a DNA vector that expresses the RNA-guided nuclease in the cell. In other examples, an RNA-guided nuclease can be delivered to a cell in an RNA that encodes the RNA-guided nuclease and expresses the nuclease in the cell. Alternatively or in addition, a gRNA targeting a gene can be delivered to a cell as a RNA, or a DNA vector that expresses the gRNA in the cell.

Delivery of an RNA-guided nuclease, gRNA, and/or an RNP may be through direct injection or cell transfection using known methods, for example, electroporation or chemical transfection. Other cell transfection methods may be used. In some instances, a Cas9 enzyme may form one RNP with all of the gRNAs targeting the TRAC gene, the β2M gene, the Reg1 gene, and the TGFBRII gene and be delivered to T cells via one electroporation event. Alternatively, a Cas9 enzyme may form two or more RNPs, which collectively include all of the gRNAs targeting the TRAC gene, the β2M gene, the Reg1 gene, and the TGFBRII gene. The multiple RNPs may be delivered to the T cells via sequential electroporation events, for example, two sequential electroporation events.

Other Gene Editing Methods

Besides the CRISPR method disclosed herein, additional gene editing methods as known in the art can also be used in making the genetically engineered T cells disclosed herein. Some examples include gene editing approaching involve zinc finger nuclease (ZFN), transcription activator-like effector nucleases (TALEN), restriction endonucleases, meganucleases homing endonucleases, and the like.

ZFNs are targeted nucleases comprising a nuclease fused to a zinc finger DNA binding domain (ZFBD), which is a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but not limited to, C2H2 zinc fingers, C3H zinc fingers, and C4 zinc fingers. A designed zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496. A selected zinc finger domain is a domain not found in nature whose production results primarily from an empirical process such as phage display, interaction trap or hybrid selection. ZFNs are described in greater detail in U.S. Pat. Nos. 7,888,121 and 7,972,854. The most recognized example of a ZFN is a fusion of the FokI nuclease with a zinc finger DNA binding domain.

A TALEN is a targeted nuclease comprising a nuclease fused to a TAL effector DNA binding domain. A “transcription activator-like effector DNA binding domain”, “TAL effector DNA binding domain”, or “TALE DNA binding domain” is a polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in US Patent Application No. 2011/0145940. The most recognized example of a TALEN in the art is a fusion polypeptide of the FokI nuclease to a TAL effector DNA binding domain.

Additional examples of targeted nucleases suitable for use as provided herein include, but are not limited to, Bxb1, phiC31, R4, PhiBT1, and Wβ/SPBc/TP901-1, whether used individually or in combination.

Any of the nucleases disclosed herein may be delivered using a vector system, including, but not limited to, plasmid vectors, DNA minicircles, retroviral vectors, lentiviral vectors, adenovirus vectors, poxvirus vectors; herpesvirus vectors and adeno-associated virus vectors, and combinations thereof.

Conventional viral and non-viral based gene transfer methods can be used to introduce nucleic acids encoding nucleases and donor templates in cells (e.g., T cells). Non-viral vector delivery systems include DNA plasmids, DNA minicircles, naked nucleic acid, and nucleic acid complexed with a delivery vehicle such as a liposome or poloxamer. Viral vector delivery systems include DNA and RNA viruses, which have either episomal or integrated genomes after delivery to the cell.

Methods of non-viral delivery of nucleic acids include electroporation, lipofection, microinjection, biolistics, virosomes, liposomes, immunoliposomes, polycation or lipid:nucleic acid conjugates, naked DNA, naked RNA, capped RNA, artificial virions, and agent-enhanced uptake of DNA. Sonoporation using, e.g., the Sonitron 2000 system (Rich-Mar) can also be used for delivery of nucleic acids. Some specific examples are provided below.

D. Delivery of Anti-CD19 CAR Construct to T Cells

In some embodiments, a nucleic acid encoding an anti-CD19 CAR can be introduced into any of the genetically engineered T cells disclosed herein by methods known to those of skill in the art. For example, a coding sequence of the anti-CD19 CAR may be cloned into a vector, which may be introduced into the genetically engineered T cells for expression of the anti-CD19 CAR. A variety of different methods known in the art can be used to introduce any of the nucleic acids or expression vectors disclosed herein into an immune effector cell. Non-limiting examples of methods for introducing nucleic acid into a cell include: lipofection, transfection (e.g., calcium phosphate transfection, transfection using highly branched organic compounds, transfection using cationic polymers, dendrimer-based transfection, optical transfection, particle-based transfection (e.g., nanoparticle transfection), or transfection using liposomes (e.g., cationic liposomes)), microinjection, electroporation, cell squeezing, sonoporation, protoplast fusion, impalefection, hydrodynamic delivery, gene gun, magnetofection, viral transfection, and nucleofection.

In specific examples, a nucleic acid encoding an anti-CD19 CAR construct can be delivered to a cell using an adeno-associated virus (AAV). AAVs are small viruses which integrate site-specifically into the host genome and can therefore deliver a transgene, such as the anti-CD19 CAR. Inverted terminal repeats (ITRs) are present flanking the AAV genome and/or the transgene of interest and serve as origins of replication. Also present in the AAV genome are rep and cap proteins which, when transcribed, form capsids which encapsulate the AAV genome for delivery into target cells. Surface receptors on these capsids which confer AAV serotype, which determines which target organs the capsids will primarily bind and thus what cells the AAV will most efficiently infect. There are twelve currently known human AAV serotypes. In some embodiments, the AAV for use in delivering the anti-CD19 CAR-coding nucleic acid is AAV serotype 6 (AAV6).

Adeno-associated viruses are among the most frequently used viruses for gene therapy for several reasons. First, AAVs do not provoke an immune response upon administration to mammals, including humans. Second, AAVs are effectively delivered to target cells, particularly when consideration is given to selecting the appropriate AAV serotype. Finally, AAVs have the ability to infect both dividing and non-dividing cells because the genome can persist in the host cell without integration. This trait makes them an ideal candidate for gene therapy.

A nucleic acid encoding a CAR can be designed to insert into a genomic site of interest in the host T cells. In some embodiments, the target genomic site can be in a safe harbor locus.

In some embodiments, a nucleic acid encoding an anti-CD19 CAR (e.g., via a donor template, which can be carried by a viral vector such as an adeno-associated viral (AAV) vector) can be designed such that it can insert into a location within a TRAC gene to disrupt the TRAC gene in the genetically engineered T cells and express the CAR polypeptide. Disruption of TRAC leads to loss of function of the endogenous TCR. For example, a disruption in the TRAC gene can be created with an endonuclease such as those described herein and one or more gRNAs targeting one or more TRAC genomic regions. Any of the gRNAs specific to a TRAC gene and the target regions disclosed herein can be used for this purpose.

In some examples, a genomic deletion in the TRAC gene and replacement by an anti-CD19 CAR coding segment can be created by homology directed repair or HDR (e.g., using a donor template, which may be part of a viral vector such as an adeno-associated viral (AAV) vector). In some embodiments, a disruption in the TRAC gene can be created with an endonuclease as those disclosed herein and one or more gRNAs targeting one or more TRAC genomic regions and inserting an anti-CD19 CAR coding segment into the TRAC gene.

A donor template as disclosed herein can contain a coding sequence for an anti-CD19 CAR. In some examples, the anti-CD19 CAR-coding sequence may be flanked by two regions of homology to allow for efficient HDR at a genomic location of interest, for example, at a TRAC gene using a gene editing method known in the art. In some examples, a CRISPR-based method can be used. In this case, both strands of the DNA at the target locus can be cut by a CRISPR Cas9 enzyme guided by gRNAs specific to the target locus. HDR then occurs to repair the double-strand break (DSB) and insert the donor DNA coding for the CAR. For this to occur correctly, the donor sequence is designed with flanking residues which are complementary to the sequence surrounding the DSB site in the target gene (hereinafter “homology arms”), such as the TRAC gene. These homology arms serve as the template for DSB repair and allow HDR to be an essentially error-free mechanism. The rate of homology directed repair (HDR) is a function of the distance between the mutation and the cut site so choosing overlapping or nearby target sites is important. Templates can include extra sequences flanked by the homologous regions or can contain a sequence that differs from the genomic sequence, thus allowing sequence editing.

Alternatively, a donor template may have no regions of homology to the targeted location in the DNA and may be integrated by NHEJ-dependent end joining following cleavage at the target site.

A donor template can be DNA or RNA, single-stranded and/or double-stranded, and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al., (1987) Proc. Natl. Acad. Sci. USA 84:4959-4963; Nehls et al., (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A donor template can be introduced into a cell as part of a vector molecule having additional sequences such as, for example, replication origins, promoters and genes encoding antibiotic resistance. Moreover, a donor template can be introduced into a cell as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

A donor template, in some embodiments, can be inserted at a site nearby an endogenous prompter (e.g., downstream or upstream) so that its expression can be driven by the endogenous promoter. In other embodiments, the donor template may comprise an exogenous promoter and/or enhancer, for example, a constitutive promoter, an inducible promoter, or tissue-specific promoter to control the expression of the CAR gene. In some embodiments, the exogenous promoter is an EF1α promoter, see, e.g., SEQ ID NO: 90 provided in Table 6 below. Other promoters may be used.

Furthermore, exogenous sequences may also include transcriptional or translational regulatory sequences, for example, promoters, enhancers, insulators, internal ribosome entry sites, sequences encoding 2A peptides and/or polyadenylation signals.

In some embodiments, a donor template for delivering an anti-CD19 CAR may be an AAV vector inserted with a nucleic acid fragment comprising the coding sequence of the anti-CD19 CAR, and optionally regulatory sequences for expression of the anti-CD19 CAR (e.g., a promoter such as the EF1a promoter provided in the sequence Table), which can be flanked by homologous arms for inserting the coding sequence and the regulatory sequences into a genomic locus of interest. In some examples, the nucleic acid fragment is inserted in the endogenous TRAC gene locus, thereby disrupting expression of the TRAC gene. In specific examples, the nucleic acid may replace a fragment in the TRAC gene, for example, a fragment comprising the nucleotide sequence of SEQ ID NO: 18. In some specific examples, the donor template for delivering the anti-CD19 CAR may comprise a nucleotide sequence of SEQ ID NO: 91, which can be inserted into a disrupted TRAC gene, for example, replacing the fragment of SEQ ID NO: 18.

E. Exemplary Anti-CD19 CAR-T Cells with Multiple Genetic Edits

It should be understood that gene disruption encompasses gene modification through gene editing (e.g., using CRISPR/Cas gene editing to insert or delete one or more nucleotides). A disrupted gene may contain one or more mutations (e.g., insertion, deletion, or nucleotide substitution, etc.) relative to the wild-type counterpart so as to substantially reduce or completely eliminate the activity of the encoded gene product. The one or more mutations may be located in a non-coding region, for example, a promoter region, a regulatory region that regulates transcription or translation; or an intron region. Alternatively, the one or more mutations may be located in a coding region (e.g., in an exon). In some instances, the disrupted gene does not express or expresses a substantially reduced level of the encoded protein. In other instances, the disrupted gene expresses the encoded protein in a mutated form, which is either not functional or has substantially reduced activity. In some embodiments, a disrupted gene is a gene that does not encode functional protein. In some embodiments, a cell that comprises a disrupted gene does not express (e.g., at the cell surface) a detectable level (e.g. by antibody, e.g., by flow cytometry) of the protein encoded by the gene. A cell that does not express a detectable level of the protein may be referred to as a knockout cell. For example, a cell having a β2M gene edit may be considered a β2M knockout cell if β2M protein cannot be detected at the cell surface using an antibody that specifically binds β2M protein. On the other hand, a cell is deemed positive (+) in expressing a surface receptor (e.g., an anti-CD19 CAR) when the surface expression of such a receptor can be detected via a routine method, e.g., by flow cytometry or immune staining.

In some embodiments, a population of genetically engineered T cells disclosed herein express an anti-CD19 CAR as those disclosed herein (e.g., comprising the amino acid sequence of SEQ ID NO:73 or SEQ ID NO: 74, a disrupted TRAC gene, a disrupted b2M gene, a disrupted Reg1 gene, and a disrupted TGFBRII gene. The nucleotide sequence encoding the anti-CD19 CAR may be inserted in the disrupted TRAC gene (e.g., replacing the site targeted by a sgRNA listed in Table 1 below).

The population of genetically engineered T cells disclosed herein may comprise a disrupted β2M gene, which comprises one or more sequences listed in Table 2, a disrupted TGFBRII gene, which comprises one or more sequences listed in Table 3, and/or a disrupted Reg1 gene, which comprises one or more sequences listed in Table 4.

Such a population of genetically engineered T cells may comprise about 50%-99% (e.g., about 55% to about 80%) CAR⁺ T cells, about 90%-99.9% (e.g., about 95% to about 99.7%) TCR⁻ T cells, about 60% to about 90% (e.g., about 70% to about 80%) β2M⁻ T cells, about 70% to about 90% (e.g., about 80% to about 90%) of TGFBRII⁻ T cells, and/or about 90% to about 98% (e.g., about 95% to about 98%) Reg1⁻ T cells. In some instances, the population of genetically engineered T cells may comprise about 80% to about 90% (e.g., about 85%) of indel frequency in the TGFBRII gene. Alternatively or in addition, the population of genetically engineered T cells may comprise about 90% to about 97% (e.g., about 95%) indel frequency in the Reg1 gene.

Any of the anti-CD19 CAR-T cells disclosed herein may be suspended in a cryopreservation solution (e.g., CryoStor® C55) to form a pharmaceutical composition. The cryopreservation solution for use in the present disclosure may also comprise adenosine, dextrose, dextran-40, lactobionic acid, sucrose, mannitol, a buffer agent such as N-)2-hydroxethyl) piperazine-N′-(2-ethanesulfonic acid) (HEPES), one or more salts (e.g., calcium chloride, magnesium chloride, potassium chloride, potassium bicarbonate, potassium phosphate, etc.), one or more base (e.g., sodium hydroxide, potassium hydroxide, etc.), or a combination thereof. Components of a cryopreservation solution may be dissolved in sterile water (injection quality). Any of the cryopreservation solution may be substantially free of serum (undetectable by routine methods).

### II. Allogeneic CAR-T Cell Therapy of CD19+ Cancer

The anti-CD19 CAR-T cells disclosed herein may be used for eliminating disease cells that express CD19 such as CD19+ cancer cells. For example, an effective amount of the anti-CD19 CAR-T cells may be administered to a subject in need of the treatment via a suitable route, e.g., intravenous infusion.

The step of administering may include the placement (e.g., transplantation) of the anti-CD19 CAR-T cells into a subject by a method or route that results in at least partial localization of the CAR-T cells at a desired site, such as a tumor site, such that a desired effect(s) can be produced. The anti-CD19 CAR-T cells can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to as long as several years, or even the life-time of the subject, i.e., long-term engraftment. For example, in some aspects described herein, an effective amount of the therapeutic T cells can be administered via a systemic route of administration, such as an intraperitoneal or intravenous route.

In some embodiments, the anti-CD19 CAR-T cells are administered systemically, which refers to the administration of a population of cells other than directly into a target site, tissue, or organ, such that it enters, instead, the subject's circulatory system and, thus, is subject to metabolism and other like processes. Suitable modes of administration include injection, infusion, instillation, or ingestion. Injection includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, the route is intravenous.

A subject may be any subject for whom diagnosis, treatment, or therapy is desired. In some embodiments, the subject is a mammal. In some embodiments, the subject is a human. The anti-CD19 CAR-T cells can be non-autologous (“non-self,” e.g., allogeneic, syngeneic or xenogeneic) to the subject. “Allogeneic” means that the anti-CD19 CAR-T cells are not derived from the subject who receives the treatment but from different individuals (donors) of the same species as the subject. A donor is an individual who is not the subject being treated. A donor is an individual who is not the patient. In some embodiments, a donor is an individual who does not have or is not suspected of having the cancer being treated. In some embodiments, multiple donors, e.g., two or more donors, are used. In some embodiments, the anti-CD19 CAR-T cell population being administered according to the methods described herein comprises allogeneic T cells obtained from one or more donors, for example, one or more healthy human donors.

An effective amount refers to the amount of the anti-CD19 CAR-T cells disclosed herein needed to prevent or alleviate at least one or more signs or symptoms of a medical condition (e.g., cancer), and relates to a sufficient amount of a composition to provide the desired effect, e.g., to treat a subject having a medical condition. An effective amount also includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

The efficacy of a treatment using the anti-CD19 CAR-T cells disclosed herein can be determined by the skilled clinician. A treatment is considered “effective”, if any one or all of the signs or symptoms of, as but one example, levels of functional target are altered in a beneficial manner (e.g., increased by at least 10%), or other clinically accepted symptoms or markers of disease (e.g., cancer) are improved or ameliorated. Efficacy can also be measured by failure of a subject to worsen as assessed by hospitalization or need for medical interventions (e.g., progression of the disease is halted or at least slowed). Methods of measuring these indicators are known to those of skill in the art and/or described herein. Treatment includes any treatment of a disease in subject and includes: (1) inhibiting the disease, e.g., arresting, or slowing the progression of symptoms; or (2) relieving the disease, e.g., causing regression of symptoms; and (3) preventing or reducing the likelihood of the development of symptoms.

An effective amount of the anti-CD19 CAR-T cells disclosed herein may comprise about 1×10⁷ anti-CD19 CAR+ cells to about 6×10⁸ anti-CD19 CAR+ cells, e.g., about 1×10⁷ cells to about 3×10⁷ cells that express the anti-CD19 CAR (CAR⁺ cells), about 3×10⁷ cells to about 1×10⁸ CAR⁺ cells, about 1×10⁸ cells to about 3×10⁸ CAR⁺ cells, or about 3×10⁸ cells to about 6×10⁸ CAR⁺ cells.

In some embodiments, the anti-CD19 CAR−T cells as disclosed herein can be used to eliminate CD19⁺ cancer cells and/or treating a CD19⁺ cancer in a human patient. In some instances, the human patient may have a B cell malignancy, for example, a refractory or relapsed B cell malignancy. As used herein, “relapsed” or “relapses” refers to a B cell malignancy such as those disclosed herein that returns following a period of complete response. Progressive disease refers to an instance when a disease worsens after the last evaluation (e.g., stable disease or partial response). Such a human patient may show one or more symptoms of B cell malignancy, e.g., unexplained weight loss, fatigue, night sweats, shortness of breath, or swollen glands. A human patient who needs the anti-CD19 CAR T cell treatment may be identified by routine medical examination, e.g., physical examination, laboratory tests, biopsy (e.g., bone marrow biopsy and/or lymph node biopsy), magnetic resonance imaging (MRI) scans, or ultrasound exams.

In some embodiments, the CD19⁺ B cell malignancy is a non-Hodgkin lymphoma (NHLs), which are a heterogeneous group of malignancies originating from B lymphocytes, T lymphocytes, or natural killer (NK) cells. The World Health Organization defines more than 60 different subcategories of NHL based on cell type in which the cancer originates, histology, mutational profiling, and protein markers on the cellular surface, and NHL is the 10th most common malignancy worldwide (Chihara et al., 2015; Trask et al., 2012). NHL accounts for 4.3% of all new cancer cases reported and is the 8th leading cause of cancer deaths in the United States. The major subtypes of NHL include diffuse large B cell lymphoma (DLBCL), chronic lymphocytic leukemia (CLL), and follicular lymphoma (FL; (Teras et al., 2016; Trask et al., 2012). CD19 expression is ubiquitous on B cell malignancies and maintained among indolent and aggressive subtypes of NHL (Scheuermann and Racila, 1995), which has contributed to the increase of development of CD19-directed therapies in these indications.

In some examples, B cell malignancies that may be treated using the methods described herein include, but are not limited to, diffuse large B cell lymphoma (DLBCL), high grade B cell lymphoma with MYC and BCL2 and/or BCL6 rearrangement, transformed follicular lymphoma (FL), grade 3b FL, or Richter's transformation of chronic lymphocytic leukemia (CLL). In some examples, the B cell malignancy is DLBCL, e.g., high grade DLBCL or DLBCL not otherwise specified (NOS). In some examples, the B cell malignancy is transformed FL or grade 3b FL. In some examples, the human patient has at least one measurable lesion that is fluorodeoxyglucose positron emission tomography (PET)-positive. In some examples, the human patient may have a refractory NHL disease with bulky presentation (high-risk subjects).

DLBCL is the most common type of NHL, accounting for 30-40% of diagnosed cases (Sehn and Gascoyne, 2015). Approximately 30-50% achieve cure with first-line chemoimmunotherapy consisting of rituximab, cyclophosphamide, doxorubicin, vincristine, and prednisone (R-CHOP; Coiffier et al., 2010; Maurer et al., 2016). However, approximately 20% are refractory to R-CHOP and 30% relapse following complete response (CR; (Maurer et al., 2016).

FL is a heterogeneous disease, usually indolent, and accounts for about 20% of reported NHL. The course is characterized by initial response to therapies followed by relapse and, at times, transformation to a more aggressive form of lymphoma. It is generally considered incurable at more advanced stages, although the 10-year survival rate is 71% for subjects with early-stage disease and 0 to 1 risk factors based on Follicular Lymphoma International Prognostic Index score (Solal-Céligny et al., 2004). FL is divided into grades 1-3 based on histologic assessment and proportion of centrocytes to centroblasts, and grade 3 is subdivided into 3a and 3b. FL grade 3b is now considered a biologically distinct entity, with frequent absence of t(14;18) and CD10 expression, and increased p53 and MUM1/IRF4 expression (Horn et al., 2011). A large retrospective analysis of more than 500 FL cases further confirmed that the clinical course of FL grade 3b is similar to FL grade 1-2, whereas FL grade 3b has a clinical course more similar to that of DLBCL (Kahl and Yang, 2016; Wahlin et al., 2012). Because of this, FL grade 3b is typically managed similarly to DLBCL (Kahl and Yang, 2016).

Combination therapies are also encompassed by the present disclosure. For example, the therapeutic T cells disclosed herein may be co-used with other therapeutic agents, for treating the same indication, or for enhancing efficacy of the therapeutic T cells and/or reducing side effects of the therapeutic T cells.

### IV. Kits

The present disclosure also provides kits for use in producing the genetically engineered T cells, the therapeutic T cells, and for therapeutic uses,

In some embodiments, a kit provided herein may comprise components for performing genetic edit of one or more of Reg1 gene, TGFBRII gene, TRAC gene, and β2M gene, and optionally a population of immune cells to which the genetic editing will be performed (e.g., a leukopak). A leukopak sample may be an enriched leukapheresis product collected from peripheral blood. It typically contains a variety of blood cells including monocytes, lymphocytes, platelets, plasma, and red cells. The components for genetically editing one or more of the target genes may comprise a suitable endonuclease such as an RNA-guided endonuclease and one or more nucleic acid guides, which direct cleavage of one or more suitable genomic sites by the endonuclease. For example, the kit may comprise a Cas enzyme such as Cas 9 and one or more gRNAs targeting a Reg1 gene, a TGFBRII gene, TRAC gene, and β2M gene. Any of the gRNAs specific to these target genes can be included in the kit.

In some embodiments, a kit provided herein may comprise a population of genetically engineered T cells as disclosed herein, and one or more components for producing the therapeutic T cells as also disclosed herein. Such components may comprise an endonuclease suitable for gene editing and a nucleic acid coding for an anti-CD19 CAR construct as disclosed herein. The CAR-coding nucleic acid may be part of a donor template as disclosed herein, which may contain homologous arms flanking the CAR-coding sequence. In some instances, the donor template may be carried by a viral vector such as an AAV vector. The kit may further comprise gRNAs specific to a TRAC gene for inserting the anti-CD19 CAR-coding sequence into the TRAC gene.

In yet other embodiments, the kit disclosed herein may comprise a population of therapeutic T cells as disclosed for the intended therapeutic purposes.

Any of the kit disclosed herein may further comprise instructions for making the therapeutic T cells, or therapeutic applications of the therapeutic T cells. In some examples, the included instructions may comprise a description of using the gene editing components to genetically engineer one or more of the target genes (e.g., Reg1, TGFBRII, TRAC gene, and β2M gene). In other examples, the included instructions may comprise a description of how to introduce a nucleic acid encoding a CAR construction into the T cells for making therapeutic T cells.

Alternatively, the kit may further comprise instructions for administration of the therapeutic T cells as disclosed herein to achieve the intended activity, e.g., eliminating disease cells targeted by the CAR expressed on the therapeutic T cells. The kit may further comprise a description of selecting a subject suitable for treatment based on identifying whether the subject is in need of the treatment. The instructions relating to the use of the therapeutic T cells described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the therapeutic T cells are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an infusion device for administration of the therapeutic T cells. A kit may have a sterile access port (for example, the container may be an intravenous solution bag or a vial having a stopper pierceable by a hypodermic injection needle). The container may also have a sterile access port.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### Sequence Tables

TABLE 1
sRNA Sequences and Target Sequences
gRNA Sequences

| Name | Unmodified Sequence | Modified Sequence |
|---|---|---|
| TRAC sgRNA | AGAGCAACAGUGCUGUGGCC | A*G*A*GCAACAGUGCUGU |
| | guuuuagagcuagaaauagc | GGCCguuuuagagcuagaa |
| | aaguuaaaauaaggcuaguc | auagcaaguuaaaauaagg |
| | cguuaucaacuugaaaaagu | cuaguccguuaucaacuug |
| | ggcaccgagucggugcUUUU | aaaaaguggcaccgagucg |
| | (SEQ ID NO: 1) | gugcU*U*U*U (SEQ ID |
| | | NO: 2) |
| TRAC sgRNA spacer | AGAGCAACAGUGCUGUGGCC | A*G*A*GCAACAGUGCUGU |
| | (SEQ ID NO: 3) | GGCC (SEQ ID NO: 4) |
| β2M sgRNA | GCUACUCUCUCUUUCUGGCC | G*C*U*ACUCUCUCUUUCU |
| | guuuuagagcuagaaauagc | GGCCguuuuagagcuagaa |
| | aaguuaaaauaaggcuaguc | auagcaaguuaaaauaagg |
| | cguuaucaacuugaaaaagu | cuaguccguuaucaacuug |
| | ggcaccgagucggugcUUUU | aaaaaguggcaccgagucg |
| | (SEQ ID NO: 5) | gugcU*U*U*U (SEQ ID |
| | | NO: 6) |
| β2M sgRNA spacer | GCUACUCUCUCUUUCUGGCC | G*C*U*ACUCUCUCUUUCU |
| | (SEQ ID NO: 7) | GGCC (SEQ ID NO: 8) |
| Reg1 sgRNA | ACGACGCGUGGGUGGCAAGC | A*C*G*ACGCGUGGGUGGC |
| | guuuuagagcuagaaauagc | AAGCguuuuagagcuagaa |
| | aaguuaaaauaaggcuaguc | auagcaaguuaaaauaagg |
| | cguuaucaacuugaaaaagu | cuaguccguuaucaacuug |
| | ggcaccgagucggugcUUUU | aaaaaguggcaccgagucg |
| | (SEQ ID NO: 9) | gugcU*U*U*U (SEQ ID |
| | | NO: 10) |
| Reg1 sgRNA spacer | ACGACGCGUGGGUGGCAAGC | A*C*G*ACGCGUGGGUGGC |
| | (SEQ ID NO: 11) | AAGC (SEQ ID NO: |
| | | 12) |
| TGFBRII sgRNA | CCCCUACCAUGACUUUAUUC | C*C*C*CUACCAUGACUUU |
| | guuuuagagcuagaaauagc | AUUCguuuuagagcuagaa |
| | aaguuaaaauaaggcuaguc | auagcaaguuaaaauaagg |
| | cguuaucaacuugaaaaagu | cuaguccguuaucaacuug |
| | ggcaccgagucggugcUUUU | aaaaaguggcaccgagucg |
| | (SEQ ID NO: 13) | gugcU*U*U*U (SEQ ID |
| | | NO: 14) |
| TGFBRII sgRNA | CCCCUACCAUGACUUUAUUC | C*C*C*CUACCAUGACUUU |
| spacer | (SEQ ID NO: 15) | AUUC (SEQ ID NO: |
| | | 16) |

Target Sequences

| | Target Sequence | Target Sequence - |
|---|---|---|
| Guide Name | (PAM) | Without PAM |
| TRAC sgRNA | AGAGCAACAGTGCTGTGGCC | AGAGCAACAGTGCTGTGGC |
| | (TGG) (SEQ ID NO: | C (SEQ ID NO: 18) |
| | 17) | |
| β2M sgRNA | GCTACTCTCTCTTTCTGGCC | GCTACTCTCTCTTTCTGGC |
| | (TGG) (SEQ ID NO: | C (SEQ ID NO: 20) |
| | 19) | |
| Reg1 sgRNA | ACGACGCGTGGGTGGCAAGC | ACGACGCGTGGGTGGCAAG |
| | (GGG) (SEQ ID NO: | C (SEQ ID NO: 22) |
| | 21) | |
| TGFBRII sgRNA | CCCCTACCATGACTTTATTC | CCCCTACCATGACTTTATT |
| | (TGG) (SEQ ID NO: | C (SEQ ID NO: 24) |
| | 23) | |

Exemplary sgRNA Formula

nnnnnnnnnnnnnnnnnnnnguuuuagagcuagaaauagcaaguuaaaauaaggc
uaguccguuaucaacuugaaaaaguggcaccgagucggugcuuuu (SEQ ID
NO: 25)
nnnnnnnnnnnnnnnnnnnnguuuuagagcuagaaauagcaaguuaaaauaaggc
uaguccguuaucaacuugaaaaaguggcaccgagucggugc (SEQ ID NO:
26)
n₍₁₇₋₃₀₎guuuuagagcuagaaauagcaaguuaaaauaaggcuaguccguuauc
aacuugaaaaaguggcaccgagucggugcu₍₁₋₈₎ (SEQ ID NO: 27)

Exemplary Cas9 sequence (spCas9)

MDKKYSIGLDIGTNSVGWAVITDEYKVPSKKFKVLGNTDRHSIKKNLIGALLFDS
GETAEATRLKRTARRRYTRRKNRICYLQEIFSNEMAKVDDSFFHRLEESFLVEED
KKHERHPIFGNIVDEVAYHEKYPTIYHLRKKLVDSTDKADLRLIYLALAHMIKFR
GHFLIEGDLNPDNSDVDKLFIQLVQTYNQLFEENPINASGVDAKAILSARLSKSR
RLENLIAQLPGEKKNGLFGNLIALSLGLTPNFKSNFDLAEDAKLQLSKDTYDDDL
DNLLAQIGDQYADLFLAAKNLSDAILLSDILRVNTEITKAPLSASMIKRYDEHHQ
DLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKI
EKILTFRIPYYVGPLARGNSRFAWMTRKSEETITPWNFEEVVDKGASAQSFIERM
TNFDKNLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKIIKDKD
FLDNEENEDILEDIVLTLTLFEDREMIEERLKTYAHLFDDKVMKQLKRRRYTGWG
RLSRKLINGIRDKQSGKTILDFLKSDGFANRNFMQLIHDDSLTFKEDIQKAQVSG
QGDSLHEHIANLAGSPAIKKGILQTVKVVDELVKVMGRHKPENIVIEMARENQTT
QKGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEVVKKMK
NYWRQLLNAKLITQRKFDNLTKAERGGLSELDKAGFIKRQLVETRQITKHVAQIL
DSRMNTKYDENDKLIREVKVITLKSKLVSDFRKDFQFYKVREINNYHHAHDAYLN
AVVGTALIKKYPKLESEFVYGDYKVYDVRKMIAKSEQEIGKATAKYFFYSNIMNF
FKTEITLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVEKGKSK
KLKSVKELLGITIMERSSFEKNPIDFLEAKGYKEVKKDLIIKLPKYSLFELENGR
KRMLASAGELQKGNELALPSKYVNFLYLASHYEKLKGSPEDNEQKQLFVEQHKHY
LDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGD (SEQ
ID NO: 28)

*2′-O-methyl phosphorothioate residue
“n” refers to the spacer sequence at the 5′ end

TABLE 2
Edited β2M Gene Sequence.

| | Sequence (Deletions indicated by dashes | SEQ ID |
|---|---|---|
| Description | (-); insertions indicated by bold) | NO: |
| β2M gene-edit | CGTGGCCTTAGCTGTGCTCGCGCTACTCTCTCTTTCT-GC | 29 |
| | CTGGAGGCTATCCAGCGTGAGTCTCTCCTACCCTCCCGCT | |
| β2M gene-edit | CGTGGCCTTAGCTGTGCTCGCGCTACTCTCTCTTTC--GC | 30 |
| | CTGGAGGCTATCCAGCGTGAGTCTCTCCTACCCTCCCGCT | |
| β2M gene-edit | CGTGGCCTTAGCTGTGCTCGCGCTACTCTCTCTTT----- | 31 |
| | CTGGAGGCTATCCAGCGTGAGTCTCTCCTACCCTCCCGCT | |
| β2M gene-edit | CGTGGCCTTAGCTGTGCTCGCGCTACTCTCTCTTTCTGGA | 32 |
| | TAGCCTGGAGGCTATCCAGCGTGAGTCTCTCCTACCCTCC | |
| | CGCT | |
| β2M gene-edit | CGTGGCCTTAGCTGTGCTCGC------------------- | 33 |
| | ------GCTATCCAGCGTGAGTCTCTCCTACCCTCCCGCT | |
| β2M gene-edit | CGTGGCCTTAGCTGTGCTCGCGCTACTCTCTCTTTCTGTG | 34 |
| | GCCTGGAGGCTATCCAGCGTGAGTCTCTCCTACCCTCCCG | |
| | CT | |

TABLE 3
Exemplary Nucleotide Sequences inD
isrupted TGFBRII Gene

| | | SEQ ID |
|---|---|---|
| Description | Gene Edited Sequence^{b} | NO |
| TGFBRII Gene-edit | CATGA-------CTGGAAGA | 35 |
| TGFBRII Gene-edit | CATGAC----TTCTGGAAGA | 36 |
| TGFBRII Gene-edit | CATGACT---TTCTGGAAGA | 37 |
| TGFBRII Gene-edit | CATGACTTTATTTCTGGAAGA | 38 |
| TGFBRII Gene-edit | CATGACTTTAATTCTGGAAGA | 39 |
| TGFBRII Gene-edit | CA-----------TGGAAGA | |
| TGFBRII Gene-edit | CATGACTT--TTCTGGAAGA | 40 |
| TGFBRII Gene-edit | CAT------------GAAGA | |
| TGFBRII Gene-edit | C------------------A | |
| TGFBRII Gene-edit | -------------------- | |
| TGFBRII Gene-edit | CATGA--------------- | |
| TGFBRII Gene-edit | CAT---------------GA | |
| TGFBRII Gene-edit | CA---------TCTGGAAGA | 41 |
| TGFBRII Gene-edit | CATGACTTT-TTCTGGAAGA | 42 |
| TGFBRII Gene-edit | CATGACTTTA-TCTGGAAGA | 43 |
| TGFBRII Gene-edit | CATGACTTT-------AAGA | 44 |
| TGFBRII Gene-edit | ----------TTCTGGAAGA | 45 |
| TGFBRII Gene-edit | CATGACTTTA--CTGGAAGA | 46 |

TABLE 4
Exemplary Nucleotide Sequences in
Disrupted Reg1 Gene

| | | SEQ ID |
|---|---|---|
| Description | Gene Edited Sequence^{b} | NO |
| Reg1 Gene-edit | GTGGGTGGCAAAGCGGGTGGT | 47 |
| Reg1 Gene-edit | GT-----------GGGTGGT | |
| Reg1 Gene-edit | -----------GCGGGTGGT | |
| Reg1 Gene-edit | GTGGGTGGC-AGCGGGTGGT | 48 |
| Reg1 Gene-edit | ---------------GTGGT | |
| Reg1 Gene-edit | GTG--------------GGT | |
| Reg1 Gene-edit | ------------CGGGTGGT | |
| Reg1 Gene-edit | -------------------- | |
| Reg1 Gene-edit | GTGGGTGGC----------- | |
| Reg1 Gene-edit | GTGGGTGGCATAGCGGGTGGT | 49 |
| Reg1 Gene-edit | GTGGGTG------------- | |
| Reg1 Gene-edit | GTGG---------------- | |
| Reg1 Gene-edit | GTGGGTGG--AGCGGGTGGT | 50 |

TABLE 5
Chimeric Antigen Receptor Sequences

| | | SEQ ID |
|---|---|---|
| Description | Sequence | NO |
| signal peptide | MLLLVTSLLLCELPHPAFLLIP | 51 |
| signal peptide | MALPVTALLLPLALLLHAARP | 52 |
| CD8a transmembrane | IYIWAPLAGTCGVLLLSLVITLY | 53 |
| domain | | |
| 4-1BB nucleotide | AAACGGGGCAGAAAGAAACTCCTGTATATATTCAAACAACCATTT | 54 |
| sequence | ATGAGACCAGTACAAACTACTCAAGAGGAAGATGGCTGTAGCTGC | |
| | CGATTTCCAGAAGAAGAAGAAGGAGGATGTGAACTG | |
| 4-1BB amino acid | KRGRKKLLYIFKQPFMRPVQTTQEEDGCSCRFPEEEEGGCEL | 55 |
| sequence | | |
| CD28 nucleotide | TCAAAGCGGAGTAGGTTGTTGCATTCCGATTACATGAATATGACT | 56 |
| sequence | CCTCGCCGGCCTGGGCCGACAAGAAAACATTACCAACCCTATGCC | |
| | CCCCCACGAGACTTCGCTGCGTACAGGTCC | |
| CD28 amino acid | SKRSRLLHSDYMNMTPRRPGPTRKHYQPYAPPRDFAAYRS | 57 |
| sequence | | |
| CD3-zeta nucleotide | CGAGTGAAGTTTTCCCGAAGCGCAGACGCTCCGGCATATCAGCAA | 58 |
| sequence | GGACAGAATCAGCTGTATAACGAACTGAATTTGGGACGCCGCGAG | |
| | GAGTATGACGTGCTTGATAAACGCCGGGGGAGAGACCCGGAAATG | |
| | GGGGGTAAACCCCGAAGAAAGAATCCCCAAGAAGGACTCTACAAT | |
| | GAACTCCAGAAGGATAAGATGGCGGAGGCCTACTCAGAAATAGGT | |
| | ATGAAGGGCGAACGACGACGGGGAAAAGGTCACGATGGCCTCTAC | |
| | CAAGGGTTGAGTACGGCAACCAAAGATACGTACGATGCACTGCAT | |
| | ATGCAGGCCCTGCCTCCCAGA | |
| CD3-zeta amino acid | RVKFSRSADAPAYQQGQNQLYNELNLGRREEYDVLDKRRGRDPEM | 59 |
| sequence | GGKPRRKNPQEGLYNELQKDKMAEAYSEIGMKGERRRGKGHDGLY | |
| | QGLSTATKDTYDALHMQALPPR | |
| anti-CD19 VL CDR1 | RASQDISKYLN | 60 |
| (Kabat) | | |
| anti-CD19 VL CDR2 | HTSRLHS | 61 |
| (Kabat) | | |
| anti-CD19 VL CDR3 | QQGNTLPYT | 62 |
| (Kabat) | | |
| anti-CD19 VH | DYGVS | 63 |
| CDR1 (Kabat) | | |
| anti-CD19 VH | VIWGSETTYYNSALKS | 64 |
| CDR2 (Kabat) | | |
| anti-CD19 VH | HYYYGGSYAMDY | 65 |
| CDR3 (Kabat) | | |
| anti-CD19 VL CDR1 | RASQDISKYLN | 66 |
| (Chothia) | | |
| anti-CD19 VL CDR2 | HTSRLHS | 67 |
| (Chothia) | | |
| anti-CD19 VL CDR3 | QQGNTLPYT | 68 |
| (Chothia) | | |
| anti-CD19 VH | GVSLPDY | 69 |
| CDR1 (Chothia) | | |
| anti-CD19 VH | WGSET | 70 |
| CDR2 (Chothia) | | |
| anti-CD19 VH | HYYYGGSYAMDY | 71 |
| CDR3 (Chothia) | | |
| Anti-CD19 CAR | ATGCTTCTTTTGGTTACGTCTCTGTTGCTTTGCGAACTTCCTCAT | 72 |
| FMC63-28Z | CCAGCGTTCTTGCTGATCCCCGATATTCAGATGACTCAGACCACC | |
| (FMC63-CD8[tm]- | AGTAGCTTGTCTGCCTCACTGGGAGACCGAGTAACAATCTCCTGC | |
| CD28[co-stimulatory | AGGGCAAGTCAAGACATTAGCAAATACCTCAATTGGTACCAGCAG | |
| domain]-CD3z) | AAGCCCGACGGAACGGTAAAACTCCTCATCTATCATACGTCAAGG | |
| | TTGCATTCCGGAGTACCGTCACGATTTTCAGGTTCTGGGAGCGGA | |
| | ACTGACTATTCCTTGACTATTTCAAACCTCGAGCAGGAGGACATT | |
| | GCGACATATTTTTGTCAACAAGGTAATACCCTCCCTTACACTTTC | |
| | GGAGGAGGAACCAAACTCGAAATTACCGGGTCCACCAGTGGCTCT | |
| | GGGAAGCCTGGCAGTGGAGAAGGTTCCACTAAAGGCGAGGTGAAG | |
| | CTCCAGGAGAGCGGCCCCGGTCTCGTTGCCCCCAGTCAAAGCCTC | |
| | TCTGTAACGTGCACAGTGAGTGGTGTATCATTGCCTGATTATGGC | |
| | GTCTCCTGGATAAGGCAGCCCCCGCGAAAGGGTCTTGAATGGCTT | |
| | GGGGTAATATGGGGCTCAGAGACAACGTATTATAACTCCGCTCTC | |
| | AAAAGTCGCTTGACGATAATAAAAGATAACTCCAAGAGTCAAGTT | |
| | TTCCTTAAAATGAACAGTTTGCAGACTGACGATACCGCTATATAT | |
| | TATTGTGCTAAACATTATTACTACGGCGGTAGTTACGCGATGGAT | |
| | TATTGGGGGCAGGGGACTTCTGTCACAGTCAGTAGTGCTGCTGCC | |
| | TTTGTCCCGGTATTTCTCCCAGCCAAACCGACCACGACTCCCGCC | |
| | CCGCGCCCTCCGACACCCGCTCCCACCATCGCCTCTCAACCTCTT | |
| | AGTCTTCGCCCCGAGGCATGCCGACCCGCCGCCGGGGGTGCTGTT | |
| | CATACGAGGGGCTTGGACTTCGCTTGTGATATTTACATTTGGGCT | |
| | CCGTTGGCGGGTACGTGCGGCGTCCTTTTGTTGTCACTCGTTATT | |
| | ACTTTGTATTGTAATCACAGGAATCGCTCAAAGCGGAGTAGGTTG | |
| | TTGCATTCCGATTACATGAATATGACTCCTCGCCGGCCTGGGCCG | |
| | ACAAGAAAACATTACCAACCCTATGCCCCCCCACGAGACTTCGCT | |
| | GCGTACAGGTCCCGAGTGAAGTTTTCCCGAAGCGCAGACGCTCCG | |
| | GCATATCAGCAAGGACAGAATCAGCTGTATAACGAACTGAATTTG | |
| | GGACGCCGCGAGGAGTATGACGTGCTTGATAAACGCCGGGGGAGA | |
| | GACCCGGAAATGGGGGGTAAACCCCGAAGAAAGAATCCCCAAGAA | |
| | GGACTCTACAATGAACTCCAGAAGGATAAGATGGCGGAGGCCTAC | |
| | TCAGAAATAGGTATGAAGGGCGAACGACGACGGGGAAAAGGTCAC | |
| | GATGGCCTCTACCAAGGGTTGAGTACGGCAACCAAAGATACGTAC | |
| | GATGCACTGCATATGCAGGCCCTGCCTCCCAGA | |
| Anti-CD19 CAR | MLLLVTSLLLCELPHPAFLLIPDIQMTQTTSSLSASLGDRVTISC | 73 |
| FMC63-28Z | RASQDISKYLNWYQQKPDGTVKLLIYHTSRLHSGVPSRFSGSGSG | (with |
| (FMC63-CD8[tm]- | TDYSLTISNLEQEDIATYFCQQGNTLPYTFGGGTKLEITGSTSGS | signal |
| CD28[co-stimulatory | GKPGSGEGSTKGEVKLQESGPGLVAPSQSLSVTCTVSGVSLPDYG | peptide) |
| domain]-CD3z) | VSWIRQPPRKGLEWLGVIWGSETTYYNSALKSRLTIIKDNSKSQV | 74 |
| Amino Acid | FLKMNSLQTDDTAIYYCAKHYYYGGSYAMDYWGQGTSVTVSSAAA | (with no |
| with signal peptide | FVPVFLPAKPTTTPAPRPPTPAPTIASQPLSLRPEACRPAAGGAV | signal |
| | HTRGLDFACDIYIWAPLAGTCGVLLLSLVITLYCNHRNRSKRSRL | peptide) |
| | LHSDYMNMTPRRPGPTRKHYQPYAPPRDFAAYRSRVKFSRSADAP | |
| | AYQQGQNQLYNELNLGRREEYDVLDKRRGRDPEMGGKPRRKNPQE | |
| | GLYNELQKDKMAEAYSEIGMKGERRRGKGHDGLYQGLSTATKDTY | |
| | DALHMQALPPR | |
| Anti-CD19 CAR | DIQMTQTTSSLSASLGDRVTISCRASQDISKYLNWYQQKPDGTVK | 75 |
| FMC63-28Z | LLIYHTSRLHSGVPSRFSGSGSGTDYSLTISNLEQEDIATYFCQQ | |
| (FMC63-CD8[tm]- | GNTLPYTFGGGTKLEITGSTSGSGKPGSGEGSTKGEVKLQESGPG | |
| CD28[co-stimulatory | LVAPSQSLSVTCTVSGVSLPDYGVSWIRQPPRKGLEWLGVIWGSE | |
| domain]-CD3z) | TTYYNSALKSRLTIIKDNSKSQVFLKMNSLQTDDTAIYYCAKHYY | |
| Amino Acid | YGGSYAMDYWGQGTSVTVSSAAAFVPVFLPAKPTTTPAPRPPTPA | |
| without signal | PTIASQPLSLRPEACRPAAGGAVHTRGLDFACDIYIWAPLAGTCG | |
| peptide | VLLLSLVITLYCNHRNRSKRSRLLHSDYMNMTPRRPGPTRKHYQP | |
| | YAPPRDFAAYRSRVKFSRSADAPAYQQGQNQLYNELNLGRREEYD | |
| | VLDKRRGRDPEMGGKPRRKNPQEGLYNELQKDKMAEAYSEIGMKG | |
| | ERRRGKGHDGLYQGLSTATKDTYDALHMQALPPR | |
| Anti-CD19 scFv | GATATTCAGATGACTCAGACCACCAGTAGCTTGTCTGCCTCACTG | 76 |
| coding sequence | GGAGACCGAGTAACAATCTCCTGCAGGGCAAGTCAAGACATTAGC | |
| | AAATACCTCAATTGGTACCAGCAGAAGCCCGACGGAACGGTAAAA | |
| | CTCCTCATCTATCATACGTCAAGGTTGCATTCCGGAGTACCGTCA | |
| | CGATTTTCAGGTTCTGGGAGCGGAACTGACTATTCCTTGACTATT | |
| | TCAAACCTCGAGCAGGAGGACATTGCGACATATTTTTGTCAACAA | |
| | GGTAATACCCTCCCTTACACTTTCGGAGGAGGAACCAAACTCGAA | |
| | ATTACCGGGTCCACCAGTGGCTCTGGGAAGCCTGGCAGTGGAGAA | |
| | GGTTCCACTAAAGGCGAGGTGAAGCTCCAGGAGAGCGGCCCCGGT | |
| | CTCGTTGCCCCCAGTCAAAGCCTCTCTGTAACGTGCACAGTGAGT | |
| | GGTGTATCATTGCCTGATTATGGCGTCTCCTGGATAAGGCAGCCC | |
| | CCGCGAAAGGGTCTTGAATGGCTTGGGGTAATATGGGGCTCAGAG | |
| | ACAACGTATTATAACTCCGCTCTCAAAAGTCGCTTGACGATAATA | |
| | AAAGATAACTCCAAGAGTCAAGTTTTCCTTAAAATGAACAGTTTG | |
| | CAGACTGACGATACCGCTATATATTATTGTGCTAAACATTATTAC | |
| | TACGGCGGTAGTTACGCGATGGATTATTGGGGGCAGGGGACTTCT | |
| | GTCACAGTCAGTAGT | |
| Anti-CD19 scFv | DIQMTQTTSSLSASLGDRVTISCRASQDISKYLNWYQQKPDGTVK | 77 |
| amino acid sequence | LLIYHTSRLHSGVPSRFSGSGSGTDYSLTISNLEQEDIATYFCQQ | |
| Linker underlined | GNTLPYTFGGGTKLEITGSTSGSGKPGSGEGSTKGEVKLQESGPG | |
| | LVAPSQSLSVTCTVSGVSLPDYGVSWIRQPPRKGLEWLGVIWGSE | |
| | TTYYNSALKSRLTIIKDNSKSQVFLKMNSLQTDDTAIYYCAKHYY | |
| | YGGSYAMDYWGQGTSVTVSS | |
| CD8a extracellular + | GCTGCTGCCTTTGTCCCGGTATTTCTCCCAGCCAAACCGACCACG | 78 |
| CD8a transmembrane + | ACTCCCGCCCCGCGCCCTCCGACACCCGCTCCCACCATCGCCTCT | |
| 5′ Linker | CAACCTCTTAGTCTTCGCCCCGAGGCATGCCGACCCGCCGCCGGG | |
| (underlined) | GGTGCTGTTCATACGAGGGGCTTGGACTTCGCTTGTGATATTTAC | |
| | ATTTGGGCTCCGTTGGCGGGTACGTGCGGCGTCCTTTTGTTGTCA | |
| | CTCGTTATTACTTTGTATTGTAATCACAGGAATCGC | |
| CD8a extracellular + | TTTGTCCCGGTATTTCTCCCAGCCAAACCGACCACGACTCCCGCC | 79 |
| CD8a transmembrane | CCGCGCCCTCCGACACCCGCTCCCACCATCGCCTCTCAACCTCTT | |
| (without linker) | AGTCTTCGCCCCGAGGCATGCCGACCCGCCGCCGGGGGTGCTGTT | |
| | CATACGAGGGGCTTGGACTTCGCTTGTGATATTTACATTTGGGCT | |
| | CCGTTGGCGGGTACGTGCGGCGTCCTTTTGTTGTCACTCGTTATT | |
| | ACTTTGTATTGTAATCACAGGAATCGC | |
| CD8a extracellular + | FVPVFLPAKPTTTPAPRPPTPAPTIASQPLSLRPEACRPAAGGAV | 80 |
| CD8a transmembrane | HTRGLDFACDIYIWAPLAGTCGVLLLSLVITLYCNHRNR | |
| Anti-CD19 VH | EVKLQESGPGLVAPSQSLSVTCTVSGVSLPDYGVSWIRQPPRKGL | 81 |
| | EWLGVIWGSETTYYNSALKSRLTIIKDNSKSQVFLKMNSLQTDDT | |
| | ATYYCAKHYYYGGSYAMDYWGQGTSVTVSS | |
| Anti-CD19 VL | DIQMTQTTSSLSASLGDRVTISCRASQDISKYLNWYQQKPDGTVK | 82 |
| | LLIYHTSRLHSGVPSRFSGSGSGTDYSLTISNLEQEDIATYFCQQ | |
| | GNTLPYTFGGGTKLEIT | |
| CD19 linker | GSTSGSGKPGSGEGSTKG | 83 |

TABLE 6
AAV Donor Template Sequences

| | | SEQ ID |
|---|---|---|
| Name | Sequence | NO: |
| Left ITR (5′ ITR) | TTGGCCACTCCCTCTCTGCGCGCTCGCTCGCTCACTGAGGCCGGG | 84 |
| | CGACCAAAGGTCGCCCGACGCCCGGGCTTTGCCCGGGCGGCCTCA | |
| | GTGAGCGAGCGAGCGCGCAGAGAGGGAGTGGCCAACTCCATCACT | |
| | AGGGGTTCCT | |
| Left ITR (5′ ITR) | CCTGCAGGCAGCTGCGCGCTCGCTCGCTCACTGAGGCCGCCCGGG | 85 |
| (alternate) | CGTCGGGCGACCTTTGGTCGCCCGGCCTCAGTGAGCGAGCGAGCG | |
| | CGCAGAGAGGGAGTGGCCAACTCCATCACTAGGGGTTCCT | |
| Right ITR (3′ ITR) | AGGAACCCCTAGTGATGGAGTTGGCCACTCCCTCTCTGCGCGCTC | 86 |
| | GCTCGCTCACTGAGGCCGCCCGGGCAAAGCCCGGGCGTCGGGCGA | |
| | CCTTTGGTCGCCCGGCCTCAGTGAGCGAGCGAGCGCGCAGAGAGG | |
| | GAGTGGCCAA | |
| Right ITR (3′ ITR) | AGGAACCCCTAGTGATGGAGTTGGCCACTCCCTCTCTGCGCGCTC | 87 |
| (alternate) | GCTCGCTCACTGAGGCCGGGCGACCAAAGGTCGCCCGACGCCCGG | |
| | GCTTTGCCCGGGCGGCCTCAGTGAGCGAGCGAGCGCGCAGCTGCC | |
| | TGCAGG | |
| TRAC-LHA (800bp) | GAGATGTAAGGAGCTGCTGTGACTTGCTCAAGGCCTTATATCGAG | 88 |
| | TAAACGGTAGTGCTGGGGCTTAGACGCAGGTGTTCTGATTTATAG | |
| | TTCAAAACCTCTATCAATGAGAGAGCAATCTCCTGGTAATGTGAT | |
| | AGATTTCCCAACTTAATGCCAACATACCATAAACCTCCCATTCTG | |
| | CTAATGCCCAGCCTAAGTTGGGGAGACCACTCCAGATTCCAAGAT | |
| | GTACAGTTTGCTTTGCTGGGCCTTTTTCCCATGCCTGCCTTTACT | |
| | CTGCCAGAGTTATATTGCTGGGGTTTTGAAGAAGATCCTATTAAA | |
| | TAAAAGAATAAGCAGTATTATTAAGTAGCCCTGCATTTCAGGTTT | |
| | CCTTGAGTGGCAGGCCAGGCCTGGCCGTGAACGTTCACTGAAATC | |
| | ATGGCCTCTTGGCCAAGATTGATAGCTTGTGCCTGTCCCTGAGTC | |
| | CCAGTCCATCACGAGCAGCTGGTTTCTAAGATGCTATTTCCCGTA | |
| | TAAAGCATGAGACCGTGACTTGCCAGCCCCACAGAGCCCCGCCCT | |
| | TGTCCATCACTGGCATCTGGACTCCAGCCTGGGTTGGGGCAAAGA | |
| | GGGAAATGAGATCATGTCCTAACCCTGATCCTCTTGTCCCACAGA | |
| | TATCCAGAACCCTGACCCTGCCGTGTACCAGCTGAGAGACTCTAA | |
| | ATCCAGTGACAAGTCTGTCTGCCTATTCACCGATTTTGATTCTCA | |
| | AACAAATGTGTCACAAAGTAAGGATTCTGATGTGTATATCACAGA | |
| | CAAAACTGTGCTAGACATGAGGTCTATGGACTTCA | |
| TRAC-RHA (800bp) | TGGAGCAACAAATCTGACTTTGCATGTGCAAACGCCTTCAACAAC | 89 |
| | AGCATTATTCCAGAAGACACCTTCTTCCCCAGCCCAGGTAAGGGC | |
| | AGCTTTGGTGCCTTCGCAGGCTGTTTCCTTGCTTCAGGAATGGCC | |
| | AGGTTCTGCCCAGAGCTCTGGTCAATGATGTCTAAAACTCCTCTG | |
| | ATTGGTGGTCTCGGCCTTATCCATTGCCACCAAAACCCTCTTTTT | |
| | ACTAAGAAACAGTGAGCCTTGTTCTGGCAGTCCAGAGAATGACAC | |
| | GGGAAAAAAGCAGATGAAGAGAAGGTGGCAGGAGAGGGCACGTGG | |
| | CCCAGCCTCAGTCTCTCCAACTGAGTTCCTGCCTGCCTGCCTTTG | |
| | CTCAGACTGTTTGCCCCTTACTGCTCTTCTAGGCCTCATTCTAAG | |
| | CCCCTTCTCCAAGTTGCCTCTCCTTATTTCTCCCTGTCTGCCAAA | |
| | AAATCTTTCCCAGCTCACTAAGTCAGTCTCACGCAGTCACTCATT | |
| | AACCCACCAATCACTGATTGTGCCGGCACATGAATGCACCAGGTG | |
| | TTGAAGTGGAGGAATTAAAAAGTCAGATGAGGGGTGTGCCCAGAG | |
| | GAAGCACCATTCTAGTTGGGGGAGCCCATCTGTCAGCTGGGAAAA | |
| | GTCCAAATAACTTCAGATTGGAATGTGTTTTAACTCAGGGTTGAG | |
| | AAAACAGCTACCTTCAGGACAAAAGTCAGGGAAGGGCTCTCTGAA | |
| | GAAATGCTACTTGAAGATACCAGCCCTACCAAGGGCAGGGAGAGG | |
| | ACCCTATAGAGGCCTGGGACAGGAGCTCAATGAGAAAGG | |
| EF1a | GGCTCCGGTGCCCGTCAGTGGGCAGAGCGCACATCGCCCACAGTC | 90 |
| | CCCGAGAAGTTGGGGGGAGGGGTCGGCAATTGAACCGGTGCCTAG | |
| | AGAAGGTGGCGCGGGGTAAACTGGGAAAGTGATGTCGTGTACTGG | |
| | CTCCGCCTTTTTCCCGAGGGTGGGGGAGAACCGTATATAAGTGCA | |
| | GTAGTCGCCGTGAACGTTCTTTTTCGCAACGGGTTTGCCGCCAGA | |
| | ACACAGGTAAGTGCCGTGTGTGGTTCCCGCGGGCCTGGCCTCTTT | |
| | ACGGGTTATGGCCCTTGCGTGCCTTGAATTACTTCCACTGGCTGC | |
| | AGTACGTGATTCTTGATCCCGAGCTTCGGGTTGGAAGTGGGTGGG | |
| | AGAGTTCGAGGCCTTGCGCTTAAGGAGCCCCTTCGCCTCGTGCTT | |
| | GAGTTGAGGCCTGGCCTGGGCGCTGGGGCCGCCGCGTGCGAATCT | |
| | GGTGGCACCTTCGCGCCTGTCTCGCTGCTTTCGATAAGTCTCTAG | |
| | CCATTTAAAATTTTTGATGACCTGCTGCGACGCTTTTTTTCTGGC | |
| | AAGATAGTCTTGTAAATGCGGGCCAAGATCTGCACACTGGTATTT | |
| | CGGTTTTTGGGGCCGCGGGCGGCGACGGGGCCCGTGCGTCCCAGC | |
| | GCACATGTTCGGCGAGGCGGGGCCTGCGAGCGCGGCCACCGAGAA | |
| | TCGGACGGGGGTAGTCTCAAGCTGGCCGGCCTGCTCTGGTGCCTG | |
| | GCCTCGCGCCGCCGTGTATCGCCCCGCCCTGGGCGGCAAGGCTGG | |
| | CCCGGTCGGCACCAGTTGCGTGAGCGGAAAGATGGCCGCTTCCCG | |
| | GCCCTGCTGCAGGGAGCTCAAAATGGAGGACGCGGCGCTCGGGAG | |
| | AGCGGGCGGGTGAGTCACCCACACAAAGGAAAAGGGCCTTTCCGT | |
| | CCTCAGCCGTCGCTTCATGTGACTCCACGGAGTACCGGGCGCCGT | |
| | CCAGGCACCTCGATTAGTTCTCGAGCTTTTGGAGTACGTCGTCTT | |
| | TAGGTTGGGGGGAGGGGTTTTATGCGATGGAGTTTCCCCACACTG | |
| | AGTGGGTGGAGACTGAAGTTAGGCCAGCTTGGCACTTGATGTAAT | |
| | TCTCCTTGGAATTTGCCCTTTTTGAGTTTGGATCTTGGTTCATTC | |
| | TCAAGCCTCAGACAGTGGTTCAAAGTTTTTTTCTTCCATTTCAGG | |
| | TGTCGTGA | |
| CD19 | GAGATGTAAGGAGCTGCTGTGACTTGCTCAAGGCCTTATATCGAG | 91 |
| LHA to RHA | TAAACGGTAGTGCTGGGGCTTAGACGCAGGTGTTCTGATTTATAG | |
| | TTCAAAACCTCTATCAATGAGAGAGCAATCTCCTGGTAATGTGAT | |
| | AGATTTCCCAACTTAATGCCAACATACCATAAACCTCCCATTCTG | |
| | CTAATGCCCAGCCTAAGTTGGGGAGACCACTCCAGATTCCAAGAT | |
| | GTACAGTTTGCTTTGCTGGGCCTTTTTCCCATGCCTGCCTTTACT | |
| | CTGCCAGAGTTATATTGCTGGGGTTTTGAAGAAGATCCTATTAAA | |
| | TAAAAGAATAAGCAGTATTATTAAGTAGCCCTGCATTTCAGGTTT | |
| | CCTTGAGTGGCAGGCCAGGCCTGGCCGTGAACGTTCACTGAAATC | |
| | ATGGCCTCTTGGCCAAGATTGATAGCTTGTGCCTGTCCCTGAGTC | |
| | CCAGTCCATCACGAGCAGCTGGTTTCTAAGATGCTATTTCCCGTA | |
| | TAAAGCATGAGACCGTGACTTGCCAGCCCCACAGAGCCCCGCCCT | |
| | TGTCCATCACTGGCATCTGGACTCCAGCCTGGGTTGGGGCAAAGA | |
| | GGGAAATGAGATCATGTCCTAACCCTGATCCTCTTGTCCCACAGA | |
| | TATCCAGAACCCTGACCCTGCCGTGTACCAGCTGAGAGACTCTAA | |
| | ATCCAGTGACAAGTCTGTCTGCCTATTCACCGATTTTGATTCTCA | |
| | AACAAATGTGTCACAAAGTAAGGATTCTGATGTGTATATCACAGA | |
| | CAAAACTGTGCTAGACATGAGGTCTATGGACTTCAGGCTCCGGTG | |
| | CCCGTCAGTGGGCAGAGCGCACATCGCCCACAGTCCCCGAGAAGT | |
| | TGGGGGGAGGGGTCGGCAATTGAACCGGTGCCTAGAGAAGGTGGC | |
| | GCGGGGTAAACTGGGAAAGTGATGTCGTGTACTGGCTCCGCCTTT | |
| | TTCCCGAGGGTGGGGGAGAACCGTATATAAGTGCAGTAGTCGCCG | |
| | TGAACGTTCTTTTTCGCAACGGGTTTGCCGCCAGAACACAGGTAA | |
| | GTGCCGTGTGTGGTTCCCGCGGGCCTGGCCTCTTTACGGGTTATG | |
| | GCCCTTGCGTGCCTTGAATTACTTCCACTGGCTGCAGTACGTGAT | |
| | TCTTGATCCCGAGCTTCGGGTTGGAAGTGGGTGGGAGAGTTCGAG | |
| | GCCTTGCGCTTAAGGAGCCCCTTCGCCTCGTGCTTGAGTTGAGGC | |
| | CTGGCCTGGGCGCTGGGGCCGCCGCGTGCGAATCTGGTGGCACCT | |
| | TCGCGCCTGTCTCGCTGCTTTCGATAAGTCTCTAGCCATTTAAAA | |
| | TTTTTGATGACCTGCTGCGACGCTTTTTTTCTGGCAAGATAGTCT | |
| | TGTAAATGCGGGCCAAGATCTGCACACTGGTATTTCGGTTTTTGG | |
| | GGCCGCGGGCGGCGACGGGGCCCGTGCGTCCCAGCGCACATGTTC | |
| | GGCGAGGCGGGGCCTGCGAGCGCGGCCACCGAGAATCGGACGGGG | |
| | GTAGTCTCAAGCTGGCCGGCCTGCTCTGGTGCCTGGCCTCGCGCC | |
| | GCCGTGTATCGCCCCGCCCTGGGCGGCAAGGCTGGCCCGGTCGGC | |
| | ACCAGTTGCGTGAGCGGAAAGATGGCCGCTTCCCGGCCCTGCTGC | |
| | AGGGAGCTCAAAATGGAGGACGCGGCGCTCGGGAGAGCGGGCGGG | |
| | TGAGTCACCCACACAAAGGAAAAGGGCCTTTCCGTCCTCAGCCGT | |
| | CGCTTCATGTGACTCCACGGAGTACCGGGCGCCGTCCAGGCACCT | |
| | CGATTAGTTCTCGAGCTTTTGGAGTACGTCGTCTTTAGGTTGGGG | |
| | GGAGGGGTTTTATGCGATGGAGTTTCCCCACACTGAGTGGGTGGA | |
| | GACTGAAGTTAGGCCAGCTTGGCACTTGATGTAATTCTCCTTGGA | |
| | ATTTGCCCTTTTTGAGTTTGGATCTTGGTTCATTCTCAAGCCTCA | |
| | GACAGTGGTTCAAAGTTTTTTTCTTCCATTTCAGGTGTCGTGACC | |
| | ACCATGCTTCTTTTGGTTACGTCTCTGTTGCTTTGCGAACTTCCT | |
| | CATCCAGCGTTCTTGCTGATCCCCGATATTCAGATGACTCAGACC | |
| | ACCAGTAGCTTGTCTGCCTCACTGGGAGACCGAGTAACAATCTCC | |
| | TGCAGGGCAAGTCAAGACATTAGCAAATACCTCAATTGGTACCAG | |
| | CAGAAGCCCGACGGAACGGTAAAACTCCTCATCTATCATACGTCA | |
| | AGGTTGCATTCCGGAGTACCGTCACGATTTTCAGGTTCTGGGAGC | |
| | GGAACTGACTATTCCTTGACTATTTCAAACCTCGAGCAGGAGGAC | |
| | ATTGCGACATATTTTTGTCAACAAGGTAATACCCTCCCTTACACT | |
| | TTCGGAGGAGGAACCAAACTCGAAATTACCGGGTCCACCAGTGGC | |
| | TCTGGGAAGCCTGGCAGTGGAGAAGGTTCCACTAAAGGCGAGGTG | |
| | AAGCTCCAGGAGAGCGGCCCCGGTCTCGTTGCCCCCAGTCAAAGC | |
| | CTCTCTGTAACGTGCACAGTGAGTGGTGTATCATTGCCTGATTAT | |
| | GGCGTCTCCTGGATAAGGCAGCCCCCGCGAAAGGGTCTTGAATGG | |
| | CTTGGGGTAATATGGGGCTCAGAGACAACGTATTATAACTCCGCT | |
| | CTCAAAAGTCGCTTGACGATAATAAAAGATAACTCCAAGAGTCAA | |
| | GTTTTCCTTAAAATGAACAGTTTGCAGACTGACGATACCGCTATA | |
| | TATTATTGTGCTAAACATTATTACTACGGCGGTAGTTACGCGATG | |
| | GATTATTGGGGGCAGGGGACTTCTGTCACAGTCAGTAGTGCTGCT | |
| | GCCTTTGTCCCGGTATTTCTCCCAGCCAAACCGACCACGACTCCC | |
| | GCCCCGCGCCCTCCGACACCCGCTCCCACCATCGCCTCTCAACCT | |
| | CTTAGTCTTCGCCCCGAGGCATGCCGACCCGCCGCCGGGGGTGCT | |
| | GTTCATACGAGGGGCTTGGACTTCGCTTGTGATATTTACATTTGG | |
| | GCTCCGTTGGCGGGTACGTGCGGCGTCCTTTTGTTGTCACTCGTT | |
| | ATTACTTTGTATTGTAATCACAGGAATCGCTCAAAGCGGAGTAGG | |
| | TTGTTGCATTCCGATTACATGAATATGACTCCTCGCCGGCCTGGG | |
| | CCGACAAGAAAACATTACCAACCCTATGCCCCCCCACGAGACTTC | |
| | GCTGCGTACAGGTCCCGAGTGAAGTTTTCCCGAAGCGCAGACGCT | |
| | CCGGCATATCAGCAAGGACAGAATCAGCTGTATAACGAACTGAAT | |
| | TTGGGACGCCGCGAGGAGTATGACGTGCTTGATAAACGCCGGGGG | |
| | AGAGACCCGGAAATGGGGGGTAAACCCCGAAGAAAGAATCCCCAA | |
| | GAAGGACTCTACAATGAACTCCAGAAGGATAAGATGGCGGAGGCC | |
| | TACTCAGAAATAGGTATGAAGGGCGAACGACGACGGGGAAAAGGT | |
| | CACGATGGCCTCTACCAAGGGTTGAGTACGGCAACCAAAGATACG | |
| | TACGATGCACTGCATATGCAGGCCCTGCCTCCCAGATAATAATAA | |
| | AATCGCTATCCATCGAAGATGGATGTGTGTTGGTTTTTTGTGTGT | |
| | GGAGCAACAAATCTGACTTTGCATGTGCAAACGCCTTCAACAACA | |
| | GCATTATTCCAGAAGACACCTTCTTCCCCAGCCCAGGTAAGGGCA | |
| | GCTTTGGTGCCTTCGCAGGCTGTTTCCTTGCTTCAGGAATGGCCA | |
| | GGTTCTGCCCAGAGCTCTGGTCAATGATGTCTAAAACTCCTCTGA | |
| | TTGGTGGTCTCGGCCTTATCCATTGCCACCAAAACCCTCTTTTTA | |
| | CTAAGAAACAGTGAGCCTTGTTCTGGCAGTCCAGAGAATGACACG | |
| | GGAAAAAAGCAGATGAAGAGAAGGTGGCAGGAGAGGGCACGTGGC | |
| | CCAGCCTCAGTCTCTCCAACTGAGTTCCTGCCTGCCTGCCTTTGC | |
| | TCAGACTGTTTGCCCCTTACTGCTCTTCTAGGCCTCATTCTAAGC | |
| | CCCTTCTCCAAGTTGCCTCTCCTTATTTCTCCCTGTCTGCCAAAA | |
| | AATCTTTCCCAGCTCACTAAGTCAGTCTCACGCAGTCACTCATTA | |
| | ACCCACCAATCACTGATTGTGCCGGCACATGAATGCACCAGGTGT | |
| | TGAAGTGGAGGAATTAAAAAGTCAGATGAGGGGTGTGCCCAGAGG | |
| | AAGCACCATTCTAGTTGGGGGAGCCCATCTGTCAGCTGGGAAAAG | |
| | TCCAAATAACTTCAGATTGGAATGTGTTTTAACTCAGGGTTGAGA | |
| | AAACAGCTACCTTCAGGACAAAAGTCAGGGAAGGGCTCTCTGAAG | |
| | AAATGCTACTTGAAGATACCAGCCCTACCAAGGGCAGGGAGAGGA | |
| | CCCTATAGAGGCCTGGGACAGGAGCTCAATGAGAAAGG | |

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1. Anti-CD19 CAR-T Cell Production and Characterization

Allogeneic human T cells that lack expression of the TRAC gene, β2M gene, Regnase-1 gene, and TGFBRII gene, and express a chimeric antigen receptor (CAR) targeting CD19 were produced. The following sgRNAs were used for the production: TA-1 (SEQ ID NO: 2) (targeting TRAC to eliminate surface expression of TCR α and β chains), β2M-1 (SEQ ID NO: 6) (targeting β2M to eliminate MHC class I surface expression), TGFBRII-5 (SEQ ID NO: 14) (targeting TGFBRII, an initiator of TGF-β signaling), and ZC3H12A-10 (SEQ ID NO: 10) (targeting Regnase-1, an immune-reaction negative regulator). CRISPR-Cas9 editing at the TRAC locus facilitated the integration of rAAV-138, which encodes the anti-CD19 CAR of SEQ ID NO:73. The anti-CD19 CAR T cells with TRAC gene, β2M gene, Regnase-1 gene and TGFBRII gene knockouts are denoted as CD19 CAR+TRAC KO+β2M KO+R/T KO.

Non-Good Manufacturing Practice (GMP) lots of CD19 CAR+TRAC KO+β2M KO+R/T KO were produced at both small scale and at manufacturing scale from 3 healthy donors each. Small-scale lots (Table 7) showed 55.6±3% CAR cell surface expression, 94.3±3.3% loss of surface TCRαβ (without TCRαβ depletion), 72.8±8.7% loss of B2M surface expression, and indel frequencies of 84.4±4.3 and 96.4±1% for TGFBRII and Regnase-1, respectively. Manufacturing-scale lots (Table 8) showed 63.3±6.2% cell surface CAR expression, 99.7±0.3% loss of surface TCRαβ (with TCRαβ depletion), 82.8±0.2% loss of β2M surface expression, and indel frequencies of 84.9±3.3 and 95.3±1.5% for TGFBR2 and Regnase-1, respectively.

TABLE 7
Small-Scale Non-GMP Lots

| T Cell Lot | Donor 1 | Donor 2 | Donor 3 | Average | S.D. |
|---|---|---|---|---|---|
| % TCRab⁻ | 91.3 | 97.8 | 93.8 | 94.3 | 3.3 |
| % β2M⁻ | 63.8 | 81.1 | 73.5 | 72.8 | 8.7 |
| % Regnase-1 indel | 97.0 | 97.0 | 95.3 | 96.4 | 1.0 |
| % TGFBR2 indel | 83.6 | 89.0 | 80.6 | 84.4 | 4.3 |
| % CAR⁺ | 57.8 | 52.1 | 56.9 | 55.6 | 3.1 |

β2M: β-2 microglobulin;
CAR: chimeric antigen receptor;
GMP: Good Manufacturing Practice;
indel: insertion/deletion;
Regnase: regulatory RNase;
S.D.: standard deviation;
TCRαβ: T cell receptor alpha and beta chains;
TGFBR2: transforming growth factor β receptor 2.

T cells from 3 individual donors were thawed and activated using TransAct beads for 48 hours in fully supplemented media (containing 5% human AB serum, IL-2, and IL-7.) On Day 2, the T cells were electroporated with RNPs containing Cas9 and gRNA targeting the Regnase-1 and TGFBRII loci. The edited cells were then seeded in fully supplemented media and cultured for 48 hours. On Day 4, the cells were electroporated with RNPs containing Cas9 and gRNAs targeting the TRAC and β2M loci. This was followed by incubation using AAV6 containing an HDR template encoding CD19 CAR. On Day 8, the cells were supplemented with IL-2 and IL-7. When cells reached a density of 3×10⁶ cells/mL (±10%), they were harvested and cryopreserved in CS5 buffer at 50×10⁶ cells/mL. Staining was performed using antibodies against TRAC and β2M proteins, whereas CAR expression was detected through staining with anti-idiotypic antibody labeled with biotin, followed by incubation with fluorescent streptavidin. The editing efficiencies of the TGFBR2 and Regnase-1 gRNAs and Cas9 were assessed by thawing the cryopreserved DP and extracting genomic DNA. This was followed by Sanger sequencing and indel analysis using tracking of indels by decomposition (TIDE) analysis.

TABLE 8
Manufacturing-Scale Non-GMP Lots

| T Cell Lot | Donor 1 | Donor 2 | Donor 3 | Average | S.D. |
|---|---|---|---|---|---|
| % TCRαβ⁻ | 99.9 | 99.9 | 99.4 | 99.7 | 0.3 |
| % β2M⁻ | 82.6 | 83.0 | 82.7 | 82.8 | 0.2 |
| % Regnase-1 indel | 97.1 | 94.3 | 94.6 | 95.3 | 1.5 |
| % TGFBR2 indel | 85.8 | 87.7 | 81.3 | 84.9 | 3.3 |
| % CAR⁺ | 56.2 | 67.6 | 66.1 | 63.3 | 6.2 |

Cells from 3 donors were produced and analyzed as described above but at manufacturing-level scale. These lots were also subjected to TCRαβ depletion when cell reached a density of 3×10⁶ cells/mL (±10%). Briefly, TCR cells were depleted using a magnetic column by incubating with anti-TCRαβ—biotin beads followed by incubation with anti-biotin beads. The TCR-depleted cells were seeded into fully supplemented media, and 16 to 20 hours after TCR depletion, the cells were harvested and cryopreserved in CS5 buffer at 50×10⁶ cells/mL.

### Example 2. TGF-β Effects on In Vitro Cell Expansion

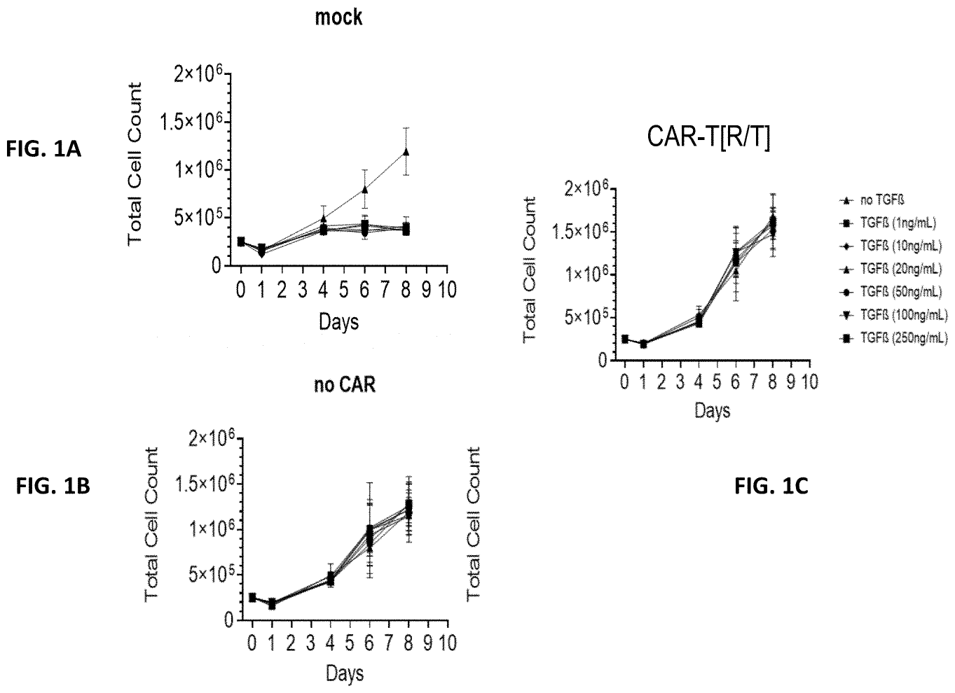

The ability of TGF-β to inhibit the growth of engineered CAR T cells with TRAC, β2M, Reg1 and TGFBRII (R/T) knockouts was assessed. The results show that TGF-β was able to inhibit the growth of mock-electroporated human T cells from 3 donors. TGF-β was unable to inhibit the growth of T cells edited to lack TGFBRII (along with disruptions in the TRAC, β2M, and Regnase-1 loci) either expressing an anti-CD19 CAR or not (no CAR) (see, FIGS. 1A-1C). The data suggest that TGFBRII disruption eliminates the inhibitory effects of TGF-β on human T cell in vitro expansion.

### Example 3. Effector Cytokine Release by Anti-CD19 CAR T Cells

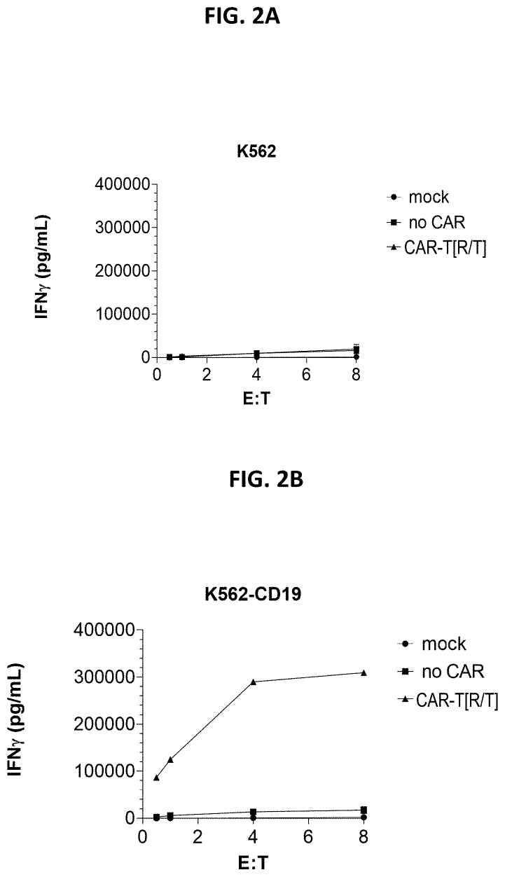

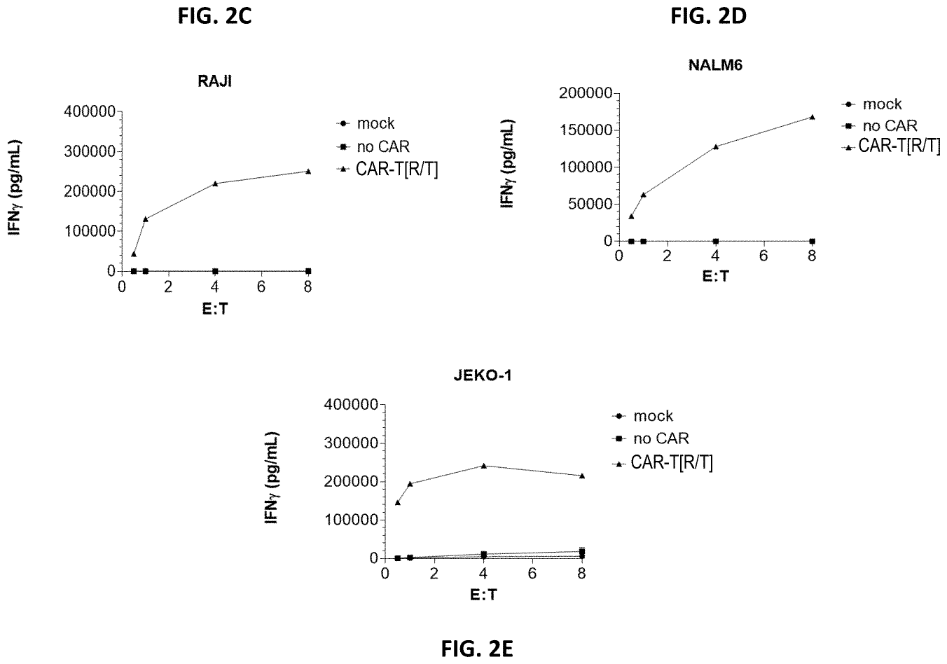

Three lots of the CD19 CAR+TRAC KO+β2M KO+R/T KO T cells were produced from 3 unique donors. Such T cells did not secrete high levels of IFN-γ in the presence of the CD19-negative K562 cell line, but secreted high levels of IFN-γ when CD19 was expressed in K562 cells (K562-CD19) as well as in the presence of 3 different human CD19-positive leukemia/lymphoma cell lines (FIGS. 2A-2E). Cells that contained the TRAC, β2M, Reg1, and TGFBRII edits except for the inserted CAR (no CAR) did not express significant levels of IFN-γ. The data suggest that the CD19 CAR+TRAC KO+β2M KO+R/T KO T cells secrete IFN-γ selectively in the presence of CD19 and additional edits do not change this specificity.

### Example 4. Cytotoxicity of Anti-CD19 CAR T Cells Against Tumor Cells

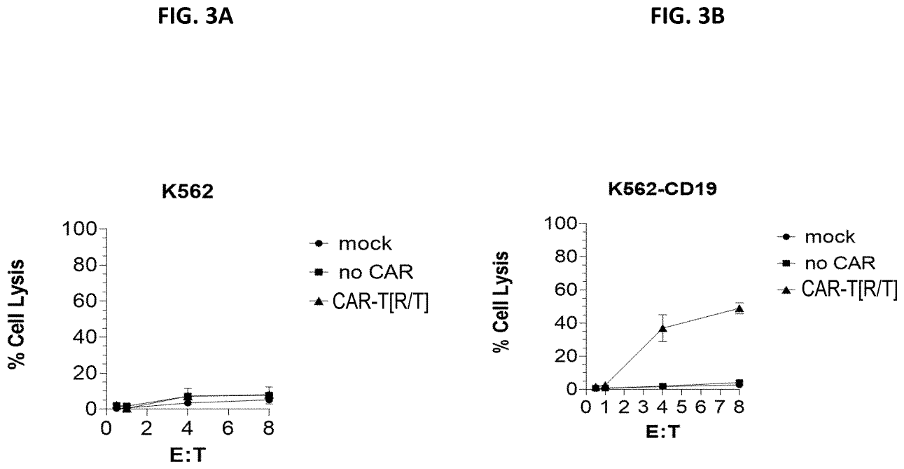

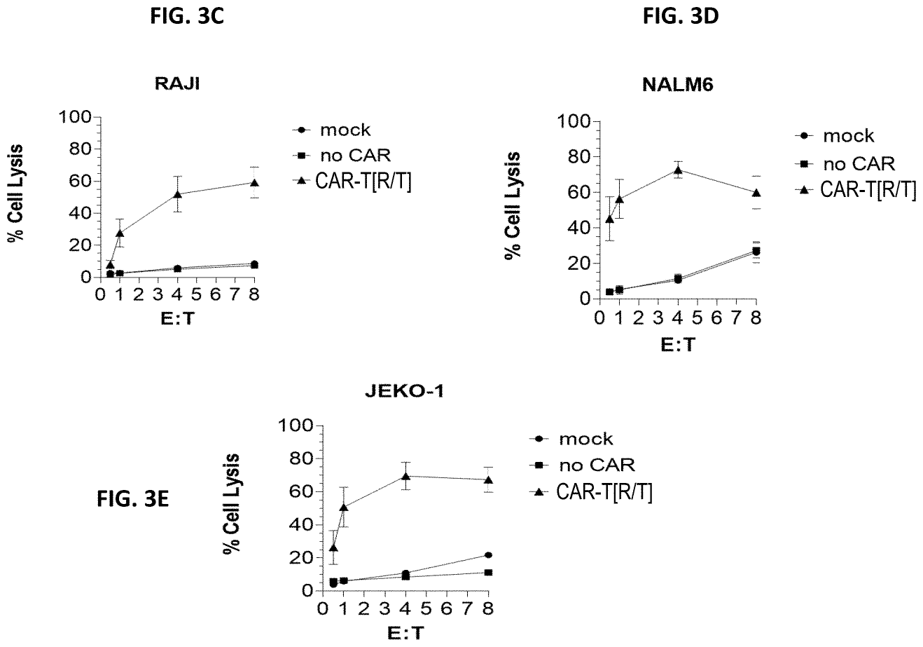

The ability of the CD19 CAR+TRAC KO+β2M KO+R/T KO T cells to kill CD19-expressing cells was assessed. Three lots of the CAR T cells were produced from 3 unique donors. Such T cells did not show levels of cytotoxic activity against the CD19-negative cell line K562 above control cells that were either TCR T cells that were mock electroporated (mock) or cells containing the TRAC, β2M, Reg1, and TGFBRII edits except for CAR insertion (no CAR). CD19 CAR+TRAC KO+β2M KO+R/T KO T cells displayed high levels of cytotoxicity in K562 cells engineered to express human CD19 (K562-CD19) as well as against 3 different CD19-expressing human leukemia/lymphoma cell lines (FIGS. 3A-3E). The data suggest that CD19 CAR+TRAC KO+β2M KO+R/T KO T cells selectively kill CD19-positive cells and additional edits do not change this specificity.

### Example 5. RNA Sequencing of Activated CAR T Cells

To assess the effects of Regnase-1 on gene expression, RNA sequencing was performed on various engineered CAR T cells, including single additional edit controls (CD19 CAR disrupted for TRAC and β2M; CD19 CAR disrupted for TRAC, β2M and TGFBRII; and CD19 CAR disrupted for TRAC, β2M, Regnase-1), and CD19 CAR+TRAC KO+β2M KO+R/T KO (CD19 CAR disrupted for TRAC, β2M, TGFBRII, and Regnase-1) in the presence and absence of CD19-positive target cells (Nalm6) at 0, 4, and 24 hours. The lists of potential Regnase-1-regulated genes are to be compared to the ones described in the literature for potential safety implications.

### Example 6. Assessment of TGFBRII and/or Regnase-1 Knockouts

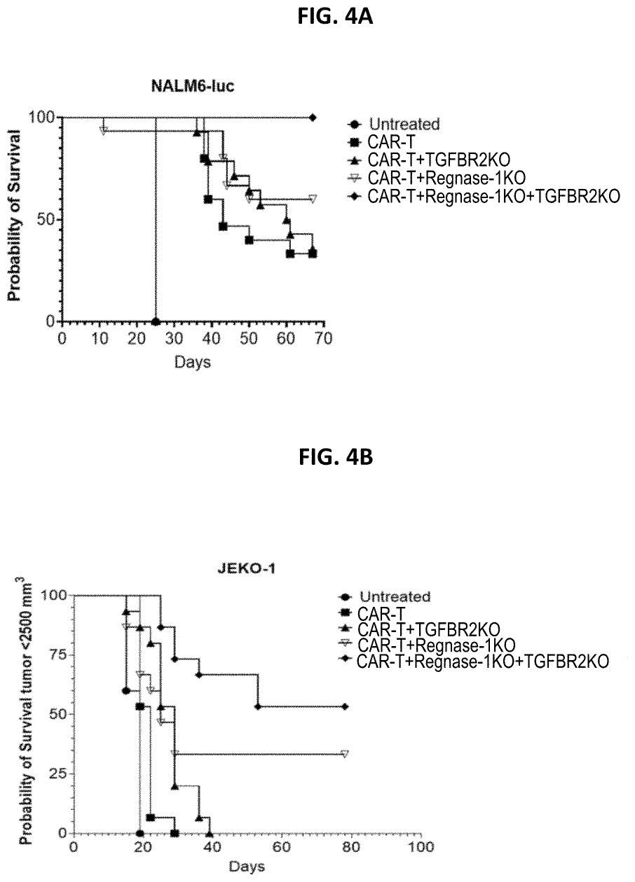

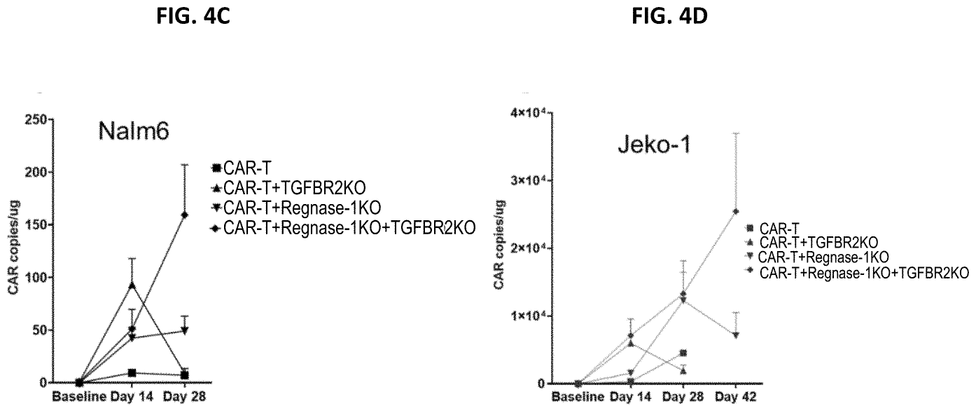

NOG mice were inoculated with either (A) Nalm6 leukemia cells intravenously or (B) Jeko-1 cells subcutaneously. Mice were infused with the indicated CAR T cells at 4×10⁶ CARP cells/mouse, and survival of mice is shown in FIGS. 4A-4B. CAR T cells were produced from 3 unique donors for both models; in each model, 5 mice were used as controls and 5 mice in each study received cells produced from each donor (15 total). ddPCR (droplet digital polymerase chain reaction) was performed on DNA isolated from peripheral blood cells of mice from the indicated CAR T cell group to detect integrated CARP human cells. The number of CAR copies per mg of DNA is indicated ±S.E.M (standard error of the mean) (FIGS. 4C-4D).

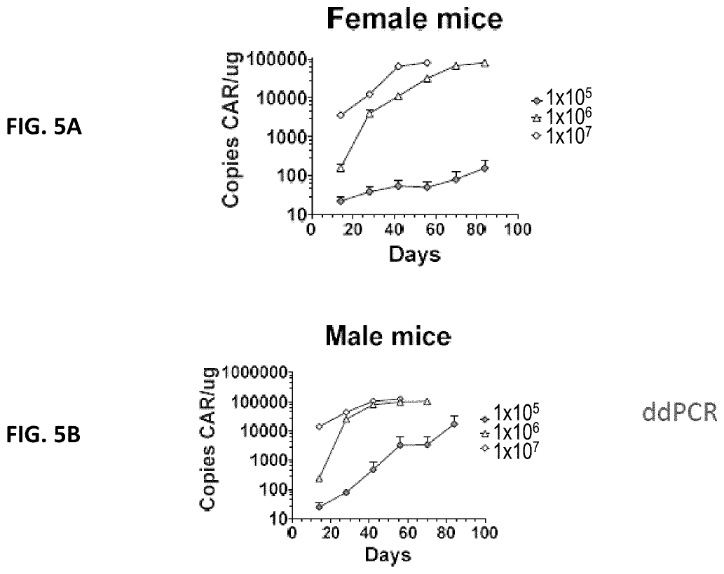

The results show that further genetic disruption of both TGFBRII and Regnase-1 in CD19 CAR+TRAC KO+β2M KO T cells synergistically increased survival of both the CD19⁺ Nalm6 leukemia and the Jeko-1 lymphoma models in immunocompromised mice (P <0.0001 log-rank [Mantel-Cox] test) for both models). Although either TGFBRII or Regnase-1 could individually increase survival in the Jeko-1 model (P<0.0006 and P=0.019, respectively), neither edit alone could increase survival relative to CD19 CAR+TRAC KO+β2M KO in both models, nor to a greater magnitude than TGFBRII/Regnase-1 double-deficient cells. This increased efficacy was correlated with synergistically increased expansion and persistence of the CD19 CAR+TRAC KO+β2M KO+R/T KO T cells relative to CD19 CAR+TRAC KO+β2M KO T cells or CD19 CAR+TRAC KO+β2M KO T cells with either just TGFBRII knockout or Regnase-1 knockout in both Nalm6 and Jeko-1 models. These data suggest that TGFBRII and Regnase-1 disruptions synergistically act to increase expansion and the functional persistence of CAR T cells. That is, TGFBRII and Regnase-1 disruption synergistically increased potency of the CD19 CAR+TRAC KO+β2M KO T cells against CD19-positive malignancies. Further assessment shows that polyclonal anti-CD19 CAR T cells with TRAC, β2M, and R/T knockouts were persistent in both female and male mice (see, FIGS. 5A-5B).

### Example 7. Therapeutic Efficacy of Anti-CD19 CAR-T Cells in CD19⁺ Leukemia and Lymphoma Xenograft Models

The CD19 CAR+TRAC KO+β2M KO+R/T KO T cells were infused into NSG mice bearing either Raji-luciferase lymphoma cells (disseminated) or Nalm6-leukemia cells (disseminated) 3 days post-inoculation of the tumor cells. For the Jeko-1 experiment, the T cells were infused into NSG mice bearing Jeko-1 lymphoma cells (subcutaneous) when tumors reached 150 mm³.

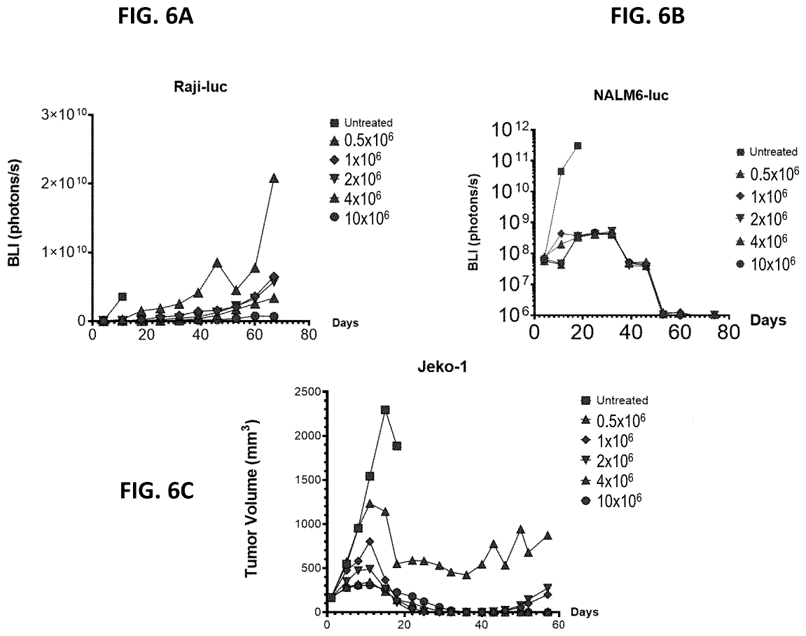

The results showed high levels of tumor control in all 3 models (FIGS. 6A-6C). Levels of leukemia or lymphoma are shown in surviving mice over time after tumor cell inoculation (Day 0). Notably, the CD19 CAR+TRAC KO+β2M KO+R/T KO T cells displayed high levels of control of the Jeko-1 lymphoma model at doses lower than a sub-efficacious dose of CD19 CAR+TRAC KO+β2M KO (<4×10⁶ CARP cells/mouse) (see, FIGS. 4A-4D).

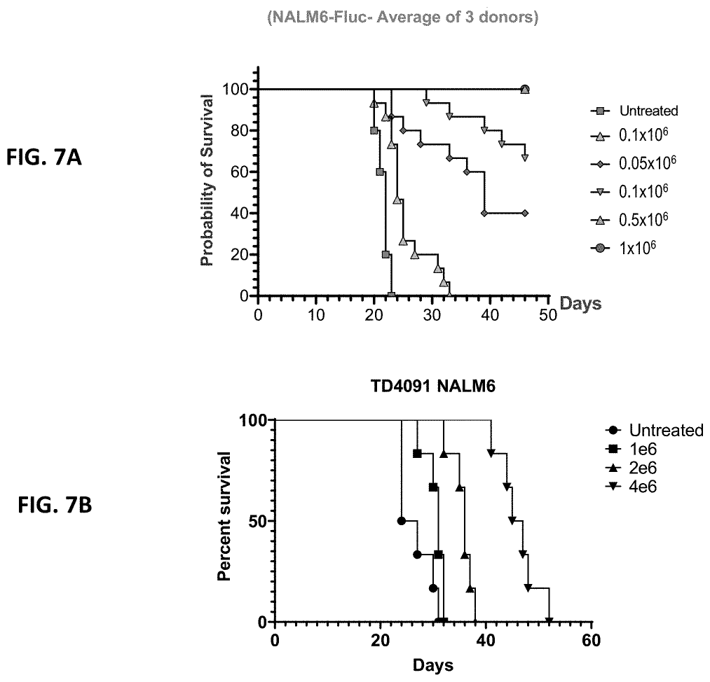

Anti-CD19 CAR T cells with TRAC, β2M, TGFBRII, and Regnase-1 knockouts are further shown to be efficacious at low doses when infused into NSG mice bearing Nalm6-leukemia cells (see, FIGS. 7A-7B).

### Example 8. Toxicology Study of CAR T Cells with Additional Edits

To assess the ability of anti-CD19 CAR T cells with TRAC, β2M, TGFBRII, and Regnase-1 knockouts to grow in the absence of human cytokines, such T cells were produced from 5 donors and 10×10⁶ cells were placed in T cell media containing 5% human serum ±IL-2/IL-7.

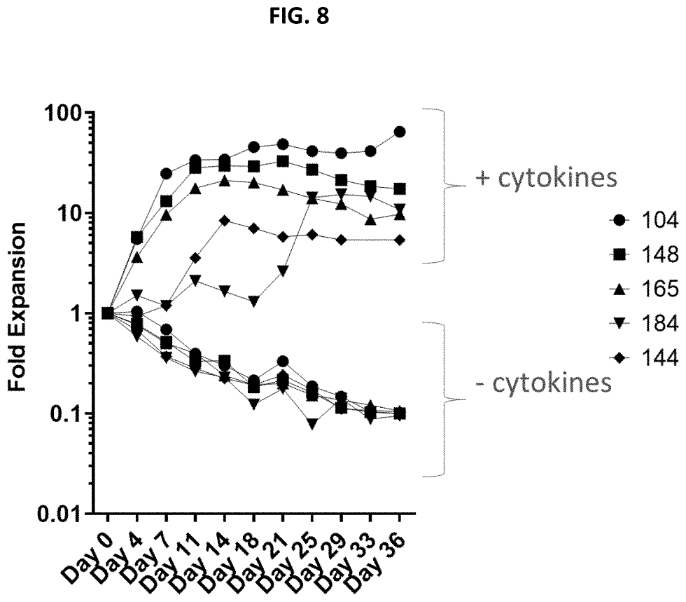

The results show that although all preparations were able to grow in the presence of cytokines, none grew in the absence of cytokines (FIG. 8). These data suggest that anti-CD19 CAR T cells with TRAC, β2M, TGFBRII, and Regnase-1 knockouts need cytokines for growth.

To assess if TGFBRII or Regnase-1 bestow altered interaction with other human tissues, anti-CD19 CART cells with TRAC, β2M, TGFBRII, and Regnase-1 knockouts along with anti-CD19 CAR T cells with TRAC and β2M knockouts will be co-cultured overnight with primary cells derived from human tissues (such as central nervous system, heart, kidney, lung, liver, bone, skin, skeletal muscle, intestine, and blood), and cytotoxicity and cytokine secretion (IFN-γ, IL-2) were measured. These experiments will determine if the additional edits of TGFBRII and Regnase-1 potentially alter reactivity with human tissue.

### Example 9. Tumorigenicity Study in Immunocompromised Mice

To support the development of an allogeneic CAR T cell, a Good Laboratory Practice (GLP)-compliant tumorigenicity study in NSG mice was conducted. In particular, a 12-week, GLP-compliant study was conducted to evaluate the tumorigenic potential of anti-CD19 CAR T cells with TRAC, β2M, TGFBRII, and Regnase-1 knockouts following a single IV slow bolus injection in NOD/SCID/IL2Rγnull (NSG) mice after total body irradiation (total irradiation dose of 200 cGy) (Table 9). Dose volume was 250 μL/mouse for all groups. Radiation was delivered at a rate of 160 cGy/minute and targeted LD_{0/140}.

TABLE 9
GLP Tumorigenicity Study Design

| | Dose Level | Total Irradiation | Number of Animals | | |
|---|---|---|---|---|---|
| Test Article | (cells/mouse) | Dose (cGy) | Male | Female | Endpoints |
| Vehicle-no RT ^{a} | 0 | 0 | 5 | 5 | Clinical |
| Vehicle-RT ^{b} | 0 | 200 | 12 | 12 | observations, body |
| CD19 CAR + | 0.5 ×10⁶ | | 12 | 12 | weight, survival, |
| TRAC KO + | | | | | hematology, serum |
| β2M KO + R/T | | | | | chemistry, |
| KO | | | | | histopathology, |
| CD19 CAR + | 1 × 10⁷ | | 12 | 12 | exposure (ddPCR, |
| TRAC KO + | | | | | IHC, cytokines) |
| β2M KO + R/T | | | | | |
| KO | | | | | |

cGy: Centigray;
ddPCR: droplet digital polymerase chain reaction;
GLP: Good Laboratory Practice;
IHC: immunohistochemistry;
PBS: phosphate-buffered saline;
RT: radiation treatment.
^{a} Animals were not irradiated and were not dosed with cells (administered PBS).
^{b} Animals were irradiated but were not dosed with cells (administered PBS).

Additional pathology and histopathological endpoints were assessed to evaluate the tolerability of the CAR T cells; however, because NSG mice do not produce B cells (the primary source of CD19 antigen), these results should be interpreted only as reflecting general off-target tolerability, with the knowledge that the CAR T cell expansion is not expected to occur in this model due to the absence of human CD19 antigen. The main endpoints of this study were survival, clinical observations, body weight measurements, and histopathology (see, Table 10). The CAR T cell exposure in mouse blood was assessed by ddPCR and in tissues by immunohistochemistry.

TABLE 10
GLP Tumorigenicity Endpoints

| Endpoints | Assay Format | Time Points |
|---|---|---|
| Clinical observations | Cageside observations | 1-2x daily |
| Peripheral blood exposure | ddPCR | Days 7, 15, 29, 43, 71, |
| | | 84 |
| Histopathology | FFPE, H&E | Day 84 |
| Peripheral blood cytokines | Luminex (IL-2, IL-5, IL-6, IL-8, IL- | Day 84 |
| (human cytokines) | 10, IFN-γ, TNF-α, GM-CSF) | |
| Tissue exposure | IHC for hCD45 and hTCR | Day 84 |

ddPCR: droplet digital polymerase chain reaction;
FFPE: formalin-fixed paraffin-embedded;
GM-CSF: granulocyte-macrophage colony-stimulating factor;
H&E: hematoxylin and eosin staining;
hCD45: human CD45 protein;
hTCR: human T cell receptor protein;
IFN-γ: interferon gamma;
IHC: immunohistochemistry;
IL: interleukin;
TNF-α: tumor necrosis factor alpha.

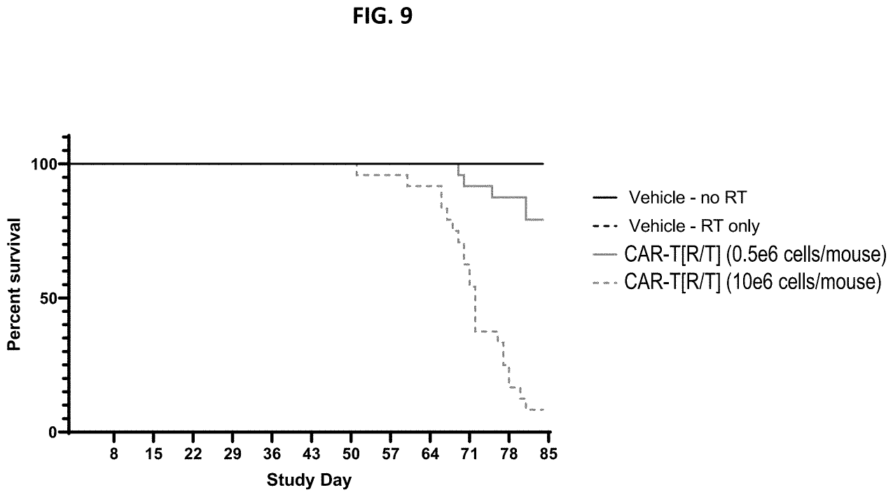

Mortality: There were 5/24 deaths (2/12 males and 3/12 females) in animals that were treated with a low dose of the CD19 CAR+TRAC KO+β2M KO+R/T KO T cells (0.5×10⁶ cells/mouse), with onset at Day 69. Mortality was also observed in animals treated with a high dose of the CART cells (1×10⁷ cells/mouse), where 12/12 males and 9/12 females were prematurely euthanized starting on Day 51, and 1/12 female was found dead on Day 66 (FIG. 9). Animals from the low-dose group were euthanized due to suspected bone fractures of the tail or hindlimb, hunched back, tucked abdomen, or difficulty with ambulation. Animals from the high-dose group were prematurely euthanized due to the aforementioned clinical signs, with additional findings of suspected bone fractures of the tail in 8/12 males and 4/9 euthanized females. Additional exhibited clinical signs from the high-dose group included but were not limited to: >20% body weight loss over a 1-week period, exudates around the eyes and nose, occasional respiratory distress (labored breathing), and decreased food and water consumption. Microscopic pathology revealed widespread mononuclear inflammation in all tissues examined. The cells in the mononuclear cell infiltrate often exhibited a positive reaction for hCD45 antigen and, to a lesser extent, hTCR antigen, indicating that they were derived from the infused human cells. All animals in both vehicle-treated groups survived to the scheduled necropsy at Day 84.

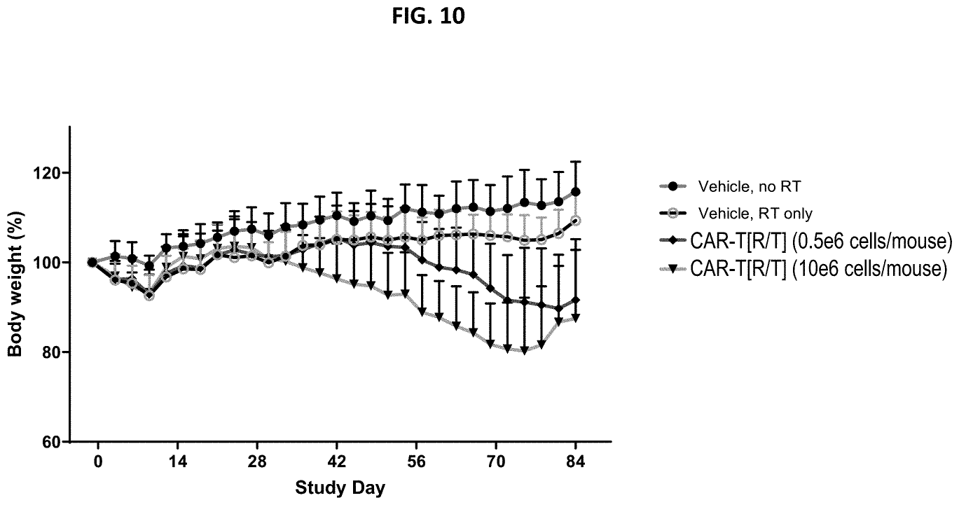

Clinical Observations: Starting on Day 32, nearly all animals treated with a low dose of the CD19 CAR+TRAC KO+β2M KO+R/T KO T cells (0.5×10⁶ cells/mouse) exhibited clinical signs, including: eye abnormalities (partly/fully closed and/or sunken), piloerection, hunched back, decreased activity, weakness, respiratory distress (labored breathing in 20/24 animals and/or increased rate in 11/24 animals), and lack of coordination. Additionally, occasional bone fractures were observed starting on Day 69 in either the hindlimb or tail in 11/24 animals. In the low-dose group, clinical signs were accompanied with body weight loss that warranted premature euthanasia of 5/24 animals between Days 69 and 81. Decreases in body weight changes were observed in animals treated with both the low dose (0.5×10⁶ cells/mouse) and high dose (1×10⁷ cells/mouse) (see, FIG. 10). There were no significant body weight changes in vehicle-treated animals.

Nearly all animals treated with the high dose (1×10⁷ cells/mouse) presented similar but more severe clinical signs with an earlier emergence. Starting on Day 6, animals exhibited hunched backs, followed by the aforementioned clinical signs of the low-dose group starting on Day 30. In addition, animals from the high-dose group exhibited suspected bone fractures in either the hindlimb or tail in 14/24 animals, tremors in 3/24 animals, and shallow respiration in 3/24 animals. These clinical signs were accompanied with severe body weight loss, leading to premature euthanasia of 22/24 animals between Days 51 and 80.

Clinical Pathology: Hematology was evaluated in animals that survived to scheduled necropsy on Day 84. Animals that received a low dose (0.5×10⁶ cells/mouse) saw a markedly higher total white blood cell count (48× in males, 36× in females) due to markedly higher neutrophil (12× in males and females) and lymphocyte (132× in males, 142× in females) counts, with minimal contributions (up to 2×) from large unstained cells and basophils when compared to the irradiated control group. Additional hematology parameters that were found to be up to 2-fold greater than in the irradiated control group included mildly higher reticulocyte counts, which resulted in minimally higher mean corpuscular volume, mildly higher red cell distribution width and hemoglobin distribution width as well as platelet distribution width.

Gross Pathology: Macroscopic observations associated with the administration of CAR T cells were present in the bone, lungs with bronchi, skin, and subcutis, and were considered related to inflammation compatible with GvHD. Enlarged spleen was also present in most animals that received a low or high dose (0.5×10⁶ cells/mouse or 1×10⁷ cells/mouse), which microscopically correlated with mild to marked infiltrate of mononuclear cells expressing moderate to severe reactivity for hCD45, and cells expressing minimal to marked reactivity for hTCR.

Microscopic Pathology: Microscopic observations of inflammation were present in the skin and subcutis, and/or lung, and/or bone with or without increased bone remodeling, and/or eye, and/or heart, and/or infusion/injection site, and/or nose, and or/tail, and/or anus of some animals that received either the low-dose (0.5×10⁶ cells/mouse) or the high-dose (1×10⁷ cells/mouse), with hematopoietic necrosis in 1 male that received the low-dose. Mononuclear cell infiltrate was present in the adrenal glands, bone marrow, brain, duodenum, large intestines, epididymides, esophagus, eyes, gall bladder, heart, infusion/injection site, kidneys, liver, lungs, various lymph nodes, mammary gland, nose, olfactory bulbs, optic nerves, ovaries, pancreas, pituitary gland, prostate gland, mandibular salivary gland, sciatic nerve, seminal vesicle, skeletal muscle (thigh), skin and subcutis, spinal cord (cervical, thoracic, lumbar), spleen, stomach, testes, thymus, thyroids, tongue, trachea, urinary bladder, uterus/cervix, and vagina of some animals that received the low- or high-dose. The cells in the mononuclear cell infiltrate often exhibited a positive reaction for hCD45 antigen and, to a lesser extent, hTCR antigen, indicating that they were derived from the infused human cells. No tumors or neoplastic lesions were found in any animals treated with the CD19 CAR+TRAC KO+β2M KO+R/T KO T cells.

Organ Weights: Organ weights were assessed from animals that survived to scheduled necropsy on Day 84. Organ weight increases related to the administration of the CD19 CAR +TRAC KO+β2M KO+R/T KO T cells were present in spleen, thymus, and adrenal glands from animals that received a low dose (0.5×10⁶ cells/mouse) and high dose (1×10⁷ cells/mouse) the CAR T cells compared to irradiated control animals and were considered to be related to mononuclear cell infiltrate due to the CAR T cell engraftment.

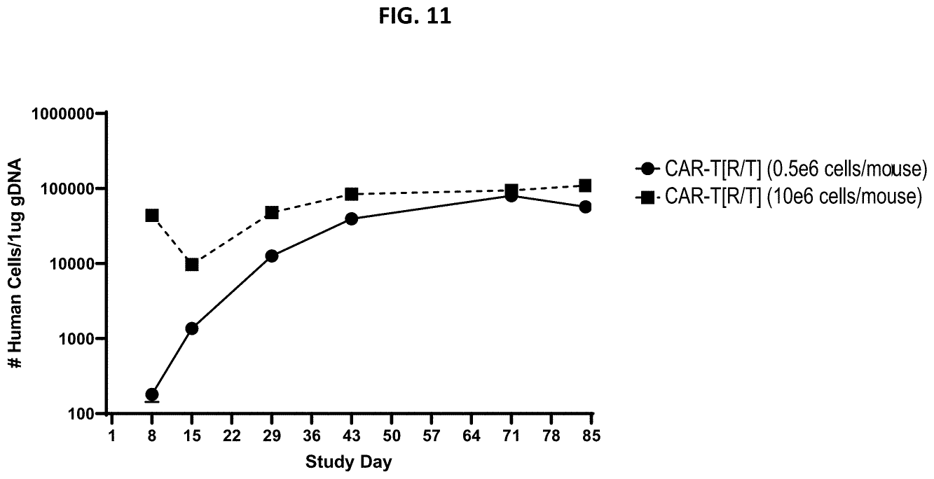

CAR T Cell Exposure in Blood: Circulating CAR T cells were detected by ddPCR at the first time point assessed (Day 8 after administration) in 12/17 animals treated with the low dose and in 19/19 animals treated with the high dose (FIG. 11). At Day 15, circulating CAR T cells were detected in all assayed animal samples and maintained through the course of the study.

### Example 10. In Vitro Mixed Lymphocyte Reaction Study

The interaction between the anti-CD19 CAR T cells with TRAC, β2M, TGFBRII, and Regnase-1 knockouts and PBMCs were assessed in MLR assays. The CAR T cells were co-cultured with PBMCs and/or subsets, and proliferation or cytotoxicity of allogeneic cells were assessed in vitro compared to co-cultures established with the CAR T cells.

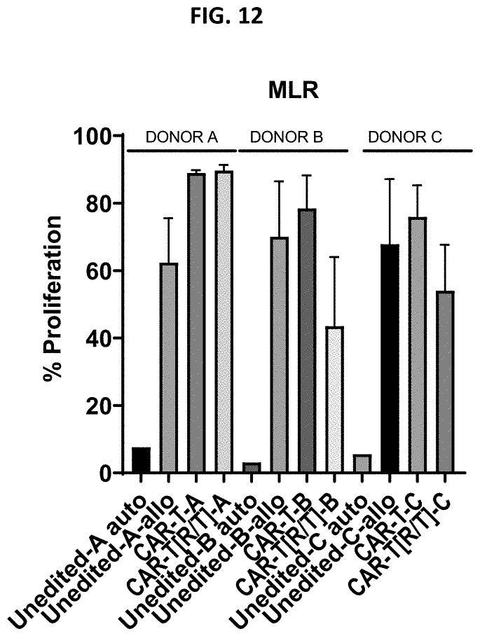

The results are shown in FIG. 12. Comparable allogeneic MLR responses were exhibited in the anti-CD19 CAR T cells with TRAC and β2M knockouts, with or without the additional knockouts of TGFBRII and Regnase-1.

### Example 11. In Vitro Natural Killer Cell Rejection

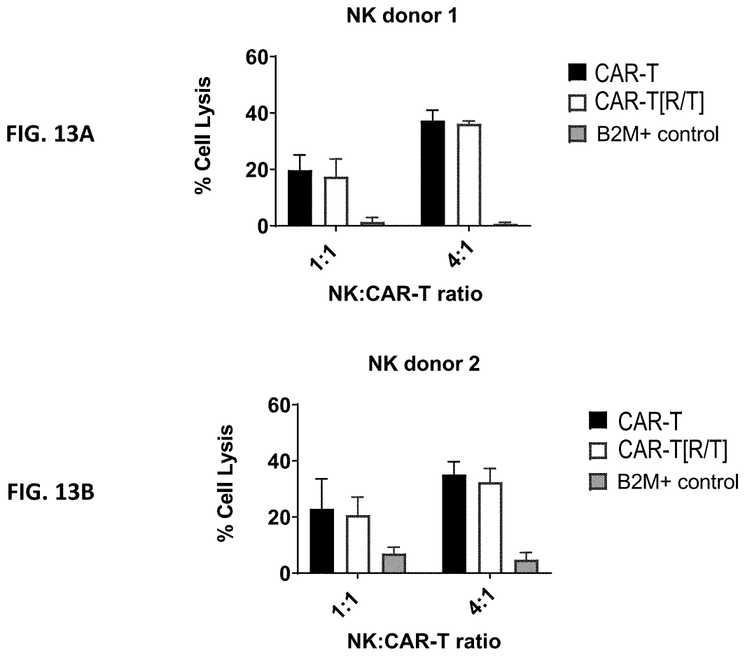

To assess the ability of allogeneic NK cells to lyse the anti-CD19 CAR T cells, the CAR T cells with TRAC and β2M knockouts and with or without the additional knockouts of TGFBRII and Regnase-1 as well as β2M⁺ control cells (CAR T cells that were not edited) from 3 unique healthy donors were co-cultured with allogeneic NK cells from 2 unique donors overnight and subjected to a flow cytometry-based cytotoxicity assay. The percentages of lysed T cells were shown in FIG. 13.

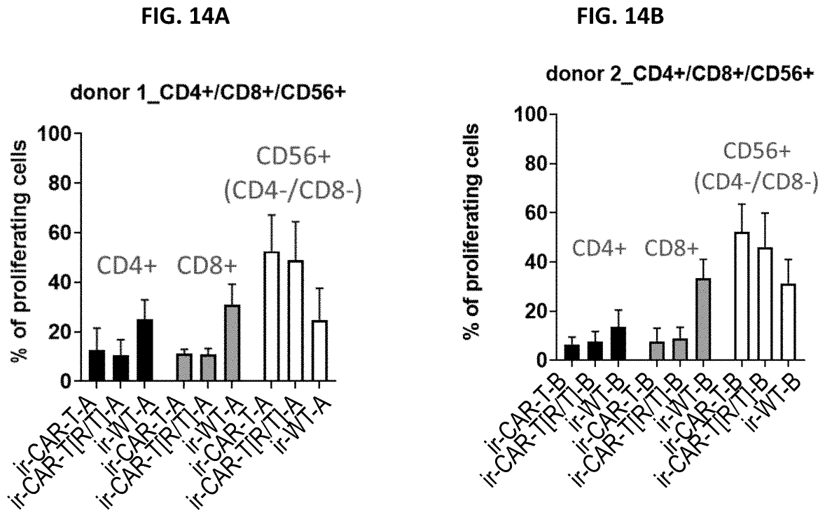

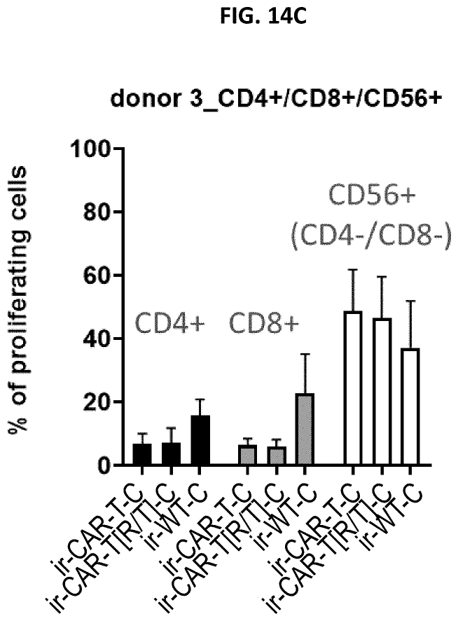

The results show that the anti-CD19 CAR T cells with TRAC and β2M knockouts and without R/T knockouts (β2M-negative average 66%) and the anti-CD19 CAR T cells with TRAC, β2M, and R/T knockouts (β2M-negative average 64%) were shown to be comparably lysed by NK cells. Control allogeneic cells from the same healthy donors as the CAR T cells that were not edited (β2M⁺) were not lysed at similar levels. The CAR T cells with the additional R/T knockouts did not show any appreciable resistance to NK-mediated attack relative the CAR T cells without the additional R/T knockouts. Additionally, the CAR T cells with or without the additional R/T knockouts showed comparable allogeneic T cell responses as well (see, FIGS. 14A-14B).

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

The term “about” as used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Where particular values are described in the application and claims, unless otherwise stated, the term “about” is implicit and in this context means within an acceptable error range for the particular value.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A population of genetically engineered T cells, comprising:
(i) a disrupted T cell receptor alpha chain constant region (TRAC) gene,
(ii) a disrupted beta-2-microglobulin (β2M) gene,
(iii) a disrupted Regnase-1 (Reg1) gene,
(iv) a disrupted Transforming Growth Factor Beta Receptor II (TGFBRII) gene, and
(v) a nucleic acid encoding a chimeric antigen receptor (CAR) that binds human CD19 (anti-CD19 CAR),
wherein the anti-CD19 CAR comprises a single chain variable fragment (scFv) that binds CD19 (anti-CD19 scFv), a co-stimulatory domain of CD28, and a CD3ζ cytoplasmic signaling domain, the anti-CD19 scFv comprising (a) a heavy chain variable region (V_{H}) that comprises the same heavy chain complementary determining regions (CDRs) as those in SEQ ID NO: 81; and (b) a light chain variable region (V_{L}) that comprises the same light chain CDRs as those in SEQ ID NO: 82; and
wherein the nucleic acid encoding the anti-CD19 CAR is inserted at the disrupted TRAC gene.

2. The population of genetically engineered T cells of claim 1, wherein at least 50% of the T cells in the population express the anti-CD19 CAR, wherein at least 90% of the T cells in the population are TCR⁻, wherein at least 60% of the T cells in the population are β2M⁻, wherein at least 80% of the T cells in the population are TGFBRII⁻, and/or wherein at least 90% of the T cells in the population are Reg1⁻.

3. The population of genetically engineered T cells of claim 2, wherein:
(a) at least 75% of the T cells express the anti-CD19 CAR;
(b) at least 99% of the T cells are TCR⁻;
(c) about 65% to about 80% of the T cells are β2M⁻;
(d) about 80% to about 90% of the T cells are TGFBRII⁻; and/or
(e) about 95% to about 97% of the T cells are Reg1⁻.

4. The population of genetically engineered T cells of claim 1, wherein the anti-CD19 scFv comprises the V_{H} comprising the amino acid sequence of SEQ ID NO: 81 and the V_{L} comprising the amino acid sequence of SEQ ID NO: 82.

5. The population of genetically engineered T cells of claim 4, wherein the anti-CD19 scFv comprises the amino acid sequence of SEQ ID NO: 77.

6. The population of genetically engineered T cells of claim 1, wherein the anti-CD19 CAR comprises the amino acid sequence of SEQ ID NO: 74.

7. The population of genetically engineered T cells of claim 1, wherein a fragment comprising the nucleotide sequence of SEQ ID NO: 18 in the TRAC gene is deleted and replaced by the nucleic acid encoding the anti-CD19 CAR.

8. The population of genetically engineered T cells of claim 7, wherein the disrupted TRAC gene comprises the nucleotide sequence of SEQ ID NO: 91.

9. The population of genetically engineered T cells of claim 1, wherein the disrupted β2M gene in the T cells comprises one or more of the nucleotide sequences listed in Table 2.

10. The population of genetically engineered T cells of claim 1, wherein the disrupted Reg1 gene in the T cells comprises one or more of the nucleotide sequences listed in Table 4.

11. The population of genetically engineered T cells of claim 1, wherein the disrupted TGFBRII gene in the T cells comprises one or more of the nucleotide sequences listed in Table 3.

12. The population of genetically engineered T cells of claim 1, wherein the T cells are primary human T cells.

13. The population of genetically engineered T cells of claim 1, wherein the T cells are derived from one or more healthy human donors.

14. A method for treating a CD19⁺ cancer, comprising administering to a subject in need thereof an effective amount of the population of genetically engineered T cells of claim 1.

15. The method of claim 14, wherein the subject is a human patient having a B cell malignancy.

16. The method of claim 15, wherein the B cell malignancy is a refractory or relapsed B cell malignancy.

17. The method of claim 15, wherein the B cell malignancy is non-Hodgkin lymphoma, which optionally is selected from the group consisting of diffuse large B cell lymphoma (DLBCL), which optionally is DLBCL not otherwise specified (NOS), high grade B cell lymphoma with MYC and BCL2 and/or BCL6 rearrangement, transformed follicular lymphoma (FL), and grade 3b FL.

18. The method of claim 14, wherein the effective amount of the population of genetically engineered T cells ranges from about 1×10⁷ to about 6×10⁸ CAR⁺ T cells.

19. A method for preparing the population of genetically engineered T cells of claim 1, the method comprising:
(a) providing a plurality of cells, which are T cells or precursor cells thereof;
(b) genetically editing the TRAC gene, the β2M gene, the Reg1 gene, and the TGFBRII gene of the plurality of cells; and
(c) delivering the nucleic acid encoding the anti-CD19 CAR into the plurality of cells, wherein the nucleic acid encoding the anti-CD19 CAR inserts into the TRAC gene,
thereby producing the population of genetically engineered T cells.

20-28. (canceled)


SEQUENCE LISTING (91 sequences)

SEQ ID NO: 1 - RNA, 100 nt, synthetic construct
  source 1..100: mol_type = other RNA
agagcaacagtgctgtggccgttttagagctagaaatagcaagttaaaataaggctagtccgttatcaacttgaaaaagtggcaccgagtcggtgctttt

SEQ ID NO: 2 - RNA, 100 nt, synthetic construct; identical to SEQ ID NO: 1
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 3 - RNA, 20 nt, synthetic construct
  source 1..20: mol_type = other RNA
agagcaacagtgctgtggcc

SEQ ID NO: 4 - RNA, 20 nt, synthetic construct
  source 1..20: mol_type = other RNA
  misc_feature 1..4: note = modified with 2'-O-methyl phosphorothioate
agagcaacagtgctgtggcc

SEQ ID NO: 5 - RNA, 100 nt, synthetic construct
  source 1..100: mol_type = other RNA
gctactctctctttctggccgttttagagctagaaatagcaagttaaaataaggctagtccgttatcaacttgaaaaagtggcaccgagtcggtgctttt

SEQ ID NO: 6 - RNA, 100 nt, synthetic construct; identical to SEQ ID NO: 5
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 7 - RNA, 20 nt, synthetic construct
  source 1..20: mol_type = other RNA
gctactctctctttctggcc

SEQ ID NO: 8 - RNA, 20 nt, synthetic construct
  source 1..20: mol_type = other RNA
  misc_feature 1..4: note = modified with 2'-O-methyl phosphorothioate
gctactctctctttctggcc

SEQ ID NO: 9 - RNA, 100 nt, synthetic construct
  source 1..100: mol_type = other RNA
acgacgcgtgggtggcaagcgttttagagctagaaatagcaagttaaaataaggctagtccgttatcaacttgaaaaagtggcaccgagtcggtgctttt

SEQ ID NO: 10 - RNA, 100 nt, synthetic construct; identical to SEQ ID NO: 9
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 11 - RNA, 20 nt, synthetic construct
  source 1..20: mol_type = other RNA
acgacgcgtgggtggcaagc

SEQ ID NO: 12 - RNA, 20 nt, synthetic construct
  source 1..20: mol_type = other RNA
  misc_feature 1..4: note = modified with 2'-O-methyl phosphorothioate
acgacgcgtgggtggcaagc

SEQ ID NO: 13 - RNA, 100 nt, synthetic construct
  source 1..100: mol_type = other RNA
cccctaccatgactttattcgttttagagctagaaatagcaagttaaaataaggctagtccgttatcaacttgaaaaagtggcaccgagtcggtgctttt

SEQ ID NO: 14 - RNA, 100 nt, synthetic construct; identical to SEQ ID NO: 13
  source 1..100: mol_type = other RNA
  misc_feature 1..4: note = modified with 2'-O-methyl phosphorothioate
  misc_feature 97..100: note = modified with 2'-O-methyl phosphorothioate

SEQ ID NO: 15 - RNA, 20 nt, synthetic construct
  source 1..20: mol_type = other RNA
cccctaccatgactttattc

SEQ ID NO: 16 - RNA, 20 nt, synthetic construct
  source 1..20: mol_type = other RNA
  misc_feature 1..4: note = modified with 2'-O-methyl phosphorothioate
cccctaccatgactttattc

SEQ ID NO: 17 - DNA, 23 bp, synthetic construct
  source 1..23: mol_type = other DNA
agagcaacagtgctgtggcctgg

SEQ ID NO: 18 - DNA, 20 bp, synthetic construct
  source 1..20: mol_type = other DNA
agagcaacagtgctgtggcc

SEQ ID NO: 19 - DNA, 23 bp, synthetic construct
  source 1..23: mol_type = other DNA
gctactctctctttctggcctgg

SEQ ID NO: 20 - DNA, 20 bp, synthetic construct
  source 1..20: mol_type = other DNA
gctactctctctttctggcc

SEQ ID NO: 21 - DNA, 23 bp, synthetic construct
  source 1..23: mol_type = other DNA
acgacgcgtgggtggcaagcggg

SEQ ID NO: 22 - DNA, 20 bp, synthetic construct
  source 1..20: mol_type = other DNA
acgacgcgtgggtggcaagc

SEQ ID NO: 23 - DNA, 23 bp, synthetic construct
  source 1..23: mol_type = other DNA
cccctaccatgactttattctgg

SEQ ID NO: 24 - DNA, 20 bp, synthetic construct
  source 1..20: mol_type = other DNA
cccctaccatgactttattc

SEQ ID NO: 25 - RNA, 100 nt, synthetic construct
  source 1..100: mol_type = other RNA
nnnnnnnnnnnnnnnnnnnngttttagagctagaaatagcaagttaaaataaggctagtccgttatcaacttgaaaaagtggcaccgagtcggtgctttt

SEQ ID NO: 26 - RNA, 96 nt, synthetic construct
  source 1..96: mol_type = other RNA
nnnnnnnnnnnnnnnnnnnngttttagagctagaaatagcaagttaaaataaggctagtccgttatcaacttgaaaaagtggcaccgagtcggtgc

SEQ ID NO: 27 - RNA, 111 nt, synthetic construct
  source 1..111: mol_type = other RNA
  misc_feature 18..30: note = n is a, c, g, u, or absent
  misc_feature 105..111: note = u or absent
nnnnnnnnnnnnnnnnnnnnnnnnnnngttttagagctagaaatagcaagttaaaataaggctagtccgttatcaacttgaaaaagtggcaccgagtcggtgctttttttt

SEQ ID NO: 28 - protein, 1368 aa, synthetic construct [sequence omitted]

SEQ ID NO: 29 - DNA, 79 bp, synthetic construct
  source 1..79: mol_type = other DNA
cgtggccttagctgtgctcgcgctactctctctttctgcctggaggctatccagcgtgagtctctcctaccctcccgct

SEQ ID NO: 30 - DNA, 78 bp, synthetic construct
  source 1..78: mol_type = other DNA
cgtggccttagctgtgctcgcgctactctctctttcgcctggaggctatccagcgtgagtctctcctaccctcccgct

SEQ ID NO: 31 - DNA, 75 bp, synthetic construct
  source 1..75: mol_type = other DNA
cgtggccttagctgtgctcgcgctactctctctttctggaggctatccagcgtgagtctctcctaccctcccgct

SEQ ID NO: 32 - DNA, 84 bp, synthetic construct
  source 1..84: mol_type = other DNA
cgtggccttagctgtgctcgcgctactctctctttctggatagcctggaggctatccagcgtgagtctctcctaccctcccgct

SEQ ID NO: 33 - DNA, 55 bp, synthetic construct
  source 1..55: mol_type = other DNA
cgtggccttagctgtgctcgcgctatccagcgtgagtctctcctaccctcccgct

SEQ ID NO: 34 - DNA, 82 bp, synthetic construct
  source 1..82: mol_type = other DNA
cgtggccttagctgtgctcgcgctactctctctttctgtggcctggaggctatccagcgtgagtctctcctaccctcccgct

SEQ ID NO: 35 - DNA, 13 bp, synthetic construct
  source 1..13: mol_type = other DNA
catgactggaaga

SEQ ID NO: 36 - DNA, 16 bp, synthetic construct
  source 1..16: mol_type = other DNA
catgacttctggaaga

SEQ ID NO: 37 - DNA, 17 bp, synthetic construct
  source 1..17: mol_type = other DNA
catgactttctggaaga

SEQ ID NO: 38 - DNA, 21 bp, synthetic construct
  source 1..21: mol_type = other DNA
catgactttatttctggaaga

SEQ ID NO: 39 - DNA, 21 bp, synthetic construct
  source 1..21: mol_type = other DNA
catgactttaattctggaaga

SEQ ID NO: 40 - DNA, 18 bp, synthetic construct
  source 1..18: mol_type = other DNA
catgacttttctggaaga

SEQ ID NO: 41 - DNA, 11 bp, synthetic construct
  source 1..11: mol_type = other DNA
catctggaaga

SEQ ID NO: 42 - DNA, 19 bp, synthetic construct
  source 1..19: mol_type = other DNA
catgactttttctggaaga

SEQ ID NO: 43 - DNA, 19 bp, synthetic construct
  source 1..19: mol_type = other DNA
catgactttatctggaaga

SEQ ID NO: 44 - DNA, 13 bp, synthetic construct
  source 1..13: mol_type = other DNA
catgactttaaga

SEQ ID NO: 45 - DNA, 10 bp, synthetic construct
  source 1..10: mol_type = other DNA
ttctggaaga

SEQ ID NO: 46 - DNA, 18 bp, synthetic construct
  source 1..18: mol_type = other DNA
catgactttactggaaga

SEQ ID NO: 47 - DNA, 21 bp, synthetic construct
  source 1..21: mol_type = other DNA
gtgggtggcaaagcgggtggt

SEQ ID NO: 48 - DNA, 19 bp, synthetic construct
  source 1..19: mol_type = other DNA
gtgggtggcagcgggtggt

SEQ ID NO: 49 - DNA, 21 bp, synthetic construct
  source 1..21: mol_type = other DNA
gtgggtggcatagcgggtggt

SEQ ID NO: 50 - DNA, 18 bp, synthetic construct
  source 1..18: mol_type = other DNA
gtgggtggagcgggtggt

SEQ ID NO: 51 - protein, 22 aa, synthetic construct
MLLLVTSLLLCELPHPAFLLIP

SEQ ID NO: 52 - protein, 21 aa, synthetic construct
MALPVTALLLPLALLLHAARP

SEQ ID NO: 53 - protein, 23 aa, synthetic construct
IYIWAPLAGTCGVLLLSLVITLY

SEQ ID NO: 54 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 55 - protein, 42 aa, synthetic construct
KRGRKKLLYIFKQPFMRPVQTTQEEDGCSCRFPEEEEGGCEL

SEQ ID NO: 56 - DNA, 120 bp, synthetic construct
  source 1..120: mol_type = other DNA
tcaaagcggagtaggttgttgcattccgattacatgaatatgactcctcgccggcctgggccgacaagaaaacattaccaaccctatgcccccccacgagacttcgctgcgtacaggtcc

SEQ ID NO: 57 - protein, 40 aa, synthetic construct
SKRSRLLHSDYMNMTPRRPGPTRKHYQPYAPPRDFAAYRS

SEQ ID NO: 58 - DNA, 336 bp, synthetic construct
  source 1..336: mol_type = other DNA
cgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 59 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 11 aa, synthetic construct
RASQDISKYLN

SEQ ID NO: 61 - protein, 7 aa, synthetic construct
HTSRLHS

SEQ ID NO: 62 - protein, 9 aa, synthetic construct
QQGNTLPYT

SEQ ID NO: 63 - protein, 5 aa, synthetic construct
DYGVS

SEQ ID NO: 64 - protein, 16 aa, synthetic construct
VIWGSETTYYNSALKS

SEQ ID NO: 65 - protein, 12 aa, synthetic construct
HYYYGGSYAMDY

SEQ ID NO: 66 - protein, 11 aa, synthetic construct
RASQDISKYLN

SEQ ID NO: 67 - protein, 7 aa, synthetic construct
HTSRLHS

SEQ ID NO: 68 - protein, 9 aa, synthetic construct
QQGNTLPYT

SEQ ID NO: 69 - protein, 7 aa, synthetic construct
GVSLPDY

SEQ ID NO: 70 - protein, 5 aa, synthetic construct
WGSET

SEQ ID NO: 71 - protein, 12 aa, synthetic construct
HYYYGGSYAMDY

SEQ ID NO: 72 - DNA, 1518 bp, synthetic construct
  source 1..1518: mol_type = other DNA
atgcttcttttggttacgtctctgttgctttgcgaacttcctcatccagcgttcttgctgatccccgatattcagatgactcagaccaccagtagcttgtctgcctcactgggagaccgagtaacaatctcctgcagggcaagtcaagacattagcaaatacctcaattggtaccagcagaagcccgacggaacggtaaaactcctcatctatcatacgtcaaggttgcattccggagtaccgtcacgattttcaggttctgggagcggaactgactattccttgactatttcaaacctcgagcaggaggacattgcgacatatttttgtcaacaaggtaataccctcccttacactttcggaggaggaaccaaactcgaaattaccgggtccaccagtggctctgggaagcctggcagtggagaaggttccactaaaggcgaggtgaagctccaggagagcggccccggtctcgttgcccccagtcaaagcctctctgtaacgtgcacagtgagtggtgtatcattgcctgattatggcgtctcctggataaggcagcccccgcgaaagggtcttgaatggcttggggtaatatggggctcagagacaacgtattataactccgctctcaaaagtcgcttgacgataataaaagataactccaagagtcaagttttccttaaaatgaacagtttgcagactgacgataccgctatatattattgtgctaaacattattactacggcggtagttacgcgatggattattgggggcaggggacttctgtcacagtcagtagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 73 - protein, 506 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - protein, 484 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 484 aa, synthetic construct; identical to SEQ ID NO: 74

SEQ ID NO: 76 - protein, 735 aa, synthetic construct
GATATTCAGATGACTCAGACCACCAGTAGCTTGTCTGCCTCACTGGGAGACCGAGTAACAATCTCCTGCAGGGCAAGTCAAGACATTAGCAAATACCTCAATTGGTACCAGCAGAAGCCCGACGGAACGGTAAAACTCCTCATCTATCATACGTCAAGGTTGCATTCCGGAGTACCGTCACGATTTTCAGGTTCTGGGAGCGGAACTGACTATTCCTTGACTATTTCAAACCTCGAGCAGGAGGACATTGCGACATATTTTTGTCAACAAGGTAATACCCTCCCTTACACTTTCGGAGGAGGAACCAAACTCGAAATTACCGGGTCCACCAGTGGCTCTGGGAAGCCTGGCAGTGGAGAAGGTTCCACTAAAGGCGAGGTGAAGCTCCAGGAGAGCGGCCCCGGTCTCGTTGCCCCCAGTCAAAGCCTCTCTGTAACGTGCACAGTGAGTGGTGTATCATTGCCTGATTATGGCGTCTCCTGGATAAGGCAGCCCCCGCGAAAGGGTCTTGAATGGCTTGGGGTAATATGGGGCTCAGAGACAACGTATTATAACTCCGCTCTCAAAAGTCGCTTGACGATAATAAAAGATAACTCCAAGAGTCAAGTTTTCCTTAAAATGAACAGTTTGCAGACTGACGATACCGCTATATATTATTGTGCTAAACATTATTACTACGGCGGTAGTTACGCGATGGATTATTGGGGGCAGGGGACTTCTGTCACAGTCAGTAGT

SEQ ID NO: 77 - protein, 245 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 78 - DNA, 261 bp, synthetic construct
  source 1..261: mol_type = other DNA
gctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgc

SEQ ID NO: 79 - DNA, 252 bp, synthetic construct
  source 1..252: mol_type = other DNA
tttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgc

SEQ ID NO: 80 - protein, 84 aa, synthetic construct
FVPVFLPAKPTTTPAPRPPTPAPTIASQPLSLRPEACRPAAGGAVHTRGLDFACDIYIWAPLAGTCGVLLLSLVITLYCNHRNR

SEQ ID NO: 81 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 82 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 83 - protein, 18 aa, synthetic construct
GSTSGSGKPGSGEGSTKG

SEQ ID NO: 84 - DNA, 145 bp, synthetic construct
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 85 - DNA, 130 bp, synthetic construct
  source 1..130: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 86 - DNA, 145 bp, synthetic construct
  source 1..145: mol_type = other DNA
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 87 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 88 - DNA, 800 bp, synthetic construct
  source 1..800: mol_type = other DNA
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttca

SEQ ID NO: 89 - DNA, 804 bp, synthetic construct
  source 1..804: mol_type = other DNA
tggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 90 - DNA, 1178 bp, synthetic construct
  source 1..1178: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 91 - DNA, 4358 bp, synthetic construct
  source 1..4358: mol_type = other DNA
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatgcttcttttggttacgtctctgttgctttgcgaacttcctcatccagcgttcttgctgatccccgatattcagatgactcagaccaccagtagcttgtctgcctcactgggagaccgagtaacaatctcctgcagggcaagtcaagacattagcaaatacctcaattggtaccagcagaagcccgacggaacggtaaaactcctcatctatcatacgtcaaggttgcattccggagtaccgtcacgattttcaggttctgggagcggaactgactattccttgactatttcaaacctcgagcaggaggacattgcgacatatttttgtcaacaaggtaataccctcccttacactttcggaggaggaaccaaactcgaaattaccgggtccaccagtggctctgggaagcctggcagtggagaaggttccactaaaggcgaggtgaagctccaggagagcggccccggtctcgttgcccccagtcaaagcctctctgtaacgtgcacagtgagtggtgtatcattgcctgattatggcgtctcctggataaggcagcccccgcgaaagggtcttgaatggcttggggtaatatggggctcagagacaacgtattataactccgctctcaaaagtcgcttgacgataataaaagataactccaagagtcaagttttccttaaaatgaacagtttgcagactgacgataccgctatatattattgtgctaaacattattactacggcggtagttacgcgatggattattgggggcaggggacttctgtcacagtcagtagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg